SEQUENCE LISTING (34 sequences)

SEQ ID NO: 1 - protein, 4 aa, synthetic construct
RGDS

SEQ ID NO: 2 - protein, 5 aa, synthetic construct
YIGSR

SEQ ID NO: 3 - protein, 5 aa, synthetic construct
IKVAV

SEQ ID NO: 4 - protein, 6 aa, synthetic construct
GFOGER

SEQ ID NO: 5 - protein, 15 aa, synthetic construct
DVDVPDGRGDSLAYG

SEQ ID NO: 6 - protein, 15 aa, synthetic construct
KLTWQELYQLKYKGI

SEQ ID NO: 7 - protein, 5 aa, synthetic construct
YGFGG

SEQ ID NO: 8 - protein, 11 aa, synthetic construct
GCGGGDWIVAG

SEQ ID NO: 9 - protein, 20 aa, synthetic construct
NSVNSKIPKACCVPTELSAI

SEQ ID NO: 10 - protein, 20 aa, synthetic construct
KIPKASSVPTELSAISTLYL

SEQ ID NO: 11 - protein, 20 aa, synthetic construct
NCKHQCTCIDGAVGCIPLCP

SEQ ID NO: 12 - protein, 15 aa, synthetic construct
TLEGTKKGHKLHLDY

SEQ ID NO: 13 - protein, 5 aa, synthetic construct
KKGHK

SEQ ID NO: 14 - protein, 4 aa, synthetic construct
SDKP

SEQ ID NO: 15 - protein, 6 aa, synthetic construct
PNDKKK

SEQ ID NO: 16 - protein, 6 aa, synthetic construct
PNDRRR

SEQ ID NO: 17 - protein, 12 aa, synthetic construct
GRPGKRGKQGQK

SEQ ID NO: 18 - protein, 10 aa, synthetic construct
RYPISRPRKR

SEQ ID NO: 19 - protein, 14 aa, synthetic construct
CGGGRYPISRPRKR

SEQ ID NO: 20 - protein, 11 aa, synthetic construct
RYPISRPRKRC

SEQ ID NO: 21 - protein, 15 aa, synthetic construct
CGGGYKTNFRRYYRF

SEQ ID NO: 22 - protein, 8 aa, synthetic construct
GPQGIWGQ

SEQ ID NO: 23 - protein, 5 aa, synthetic construct
PQGLA

SEQ ID NO: 24 - protein, 8 aa, synthetic construct
ELAPLRAP

SEQ ID NO: 25 - protein, 8 aa, synthetic construct
FPLRMRDW

SEQ ID NO: 26 - protein, 5 aa, synthetic construct
KHGKV

SEQ ID NO: 27 - protein, 8 aa, synthetic construct
EGTKKGHK

SEQ ID NO: 28 - protein, 8 aa, synthetic construct
KKGHKLHL

SEQ ID NO: 29 - protein, 11 aa, synthetic construct
HPVGLLARNRV

SEQ ID NO: 30 - protein, 6 aa, synthetic construct
PVGLIG

SEQ ID NO: 31 - protein, 14 aa, synthetic construct
CGGGPALLREGGGC

SEQ ID NO: 32 - protein, 16 aa, synthetic construct
CRDTEGEARGSVIDRC

SEQ ID NO: 33 - protein, 11 aa, synthetic construct
GCGYGRGDSPG

SEQ ID NO: 34 - protein, 5 aa, synthetic construct
DWIVA

(19) United States
(12) Patent Application Publication
(10) Pub. No.: US 2026/0022213 A1
(43) Pub. Date: Jan. 22, 2026

(54) HYDROGELS AND METHODS OF USING THE SAME

(71) Applicants: Rowan University, Glassboro, NJ (US); The Cooper Health System, Camden, NJ (US)

(72) Inventors: Sebastian L. Vega, Sewell, NJ (US); Kirstene Gultian, Glassboro, NJ (US); Tae Won B. Kim, Camden, NJ (US)

(73) Assignees: Rowan University, Glassboro, NJ (US); The Cooper Health System, Camden, NJ (US)

(21) Appl. No.: 18/996,683

(22) PCT Filed: Jul. 21, 2023
(86) PCT No.: PCT/US2023/070731

§ 371 Date: Jan. 17, 2025

Related U.S. Application Data
(60) Provisional application No. 63/391,422, filed on Jul. 22, 2022.

(51) Int. Cl. C08J 3/075; A61L 27/52; A61L 27/54; C08J 3/24

(52) U.S. Cl. CPC C08J 3/075; A61L 27/52; A61L 27/54; C08J 3/246; A61L 2300/414; A61L 2400/06; A61L 2430/02; C08J 2305/08

(57) ABSTRACT

Described herein is a hydrogel including a hydrogel polymer; a crosslinker crosslinking the hydrogel polymer; a biomolecule attached to the hydrogel polymer; and water. In the hydrogel, the biomolecule is attached to the hydrogel polymer through an acrylic linker attached to a hydroxyl group in the hydrogel polymer. Also described is a hyaluronic acid (HA)-based hydrogel including a first HA polymer comprising a first crosslinker; a second HA polymer comprising a second crosslinker; a biomolecule attached to the first HA polymer or the second HA polymer through an acrylic linker attached to a hydroxyl group in the first HA polymer or the second HA polymer; and water. Also described is a method of producing the HA-based hydrogel, as well as a method of regenerating tissues using the hydrogels.

### CROSS-REFERENCE TO RELATED APPLICATIONS

The present application claims priority under 35 U.S.C. § 119 (e) to U.S. Provisional Patent Application No. 63/391,422, filed Jul. 22, 2022, which is incorporated herein by reference in its entirety.

### STATEMENT REGARDING FEDERALLY SPONSORED RESEARCH

This invention was made with government support under R21DC018818 awarded by the National Institutes of Health. The government has certain rights in the invention.

### SEQUENCE LISTING

The XML text file named “370431-1040WO1 (00282)_Sequence Listing.xml” created on Jun. 13, 2013, comprising 29.7 Kbytes, is hereby incorporated by reference in its entirety.

### BACKGROUND

Hydrogels are three-dimensional, highly hydrated crosslinked polymer networks. They have been used in various biomedical applications such as tissue engineering, drug delivery, and regenerative medicine.

The hydrogels currently available for tissue regenerations have various shortcomings. For example, the mechanical properties of currently existing hydrogels are not sufficiently tunable. Further, it is often necessary to attach biomolecules to the polymer networks for regenerative medicine purposes, but the existing chemistry for this attachment often has undesirable impact on mechanical properties of hydrogels.

Thus, there is a need to develop novel hydrogels that have easily tunable mechanical parameters. Such hydrogels should also allow for biomolecules such as proteins and peptides to be attached to them, without affecting the mechanical parameters of the hydrogels. The present invention addresses this need.

### SUMMARY

In some aspects, the present invention is directed to the following non-limiting embodiments:

In some aspects, the present invention is directed to a hydrogel.

In some embodiments, the hydrogel comprises: a hydrogel polymer; a crosslinker crosslinking the hydrogel polymer; a biomolecule attached to the hydrogel polymer; and water.

In some embodiments, the biomolecule is attached to the hydrogel polymer through an acrylic linker attached to a hydroxyl group in the hydrogel polymer.

In some embodiments, the hydrogel polymer comprises at least one selected from the group consisting of an alginate polymer, an α,β-poly(N-hydroxyethyl)-DL-aspartamide polymer, a chitosan polymer, a chondroitin sulfate polymer, a collagen/gelatin polymer, an elastin polymer, a fibrin polymer, a heparin polymer, a hyaluronic acid polymer, and a poly(vinyl alcohol) polymer.

In some embodiments, the amount of the hydrogel polymer in the hydrogel ranges from about 1% w/v to about 10% w/v based on a total volume of the hydrogel.

In some embodiments, the crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

In some embodiments, the molar % of repeating units in the hydrogel polymer that are directly attached to the crosslinker ranges from about 10% to about 60%.

In some embodiments, the acrylic linker is attached to the hydroxyl group in the hydrogel polymer via an esterification reaction between an acrylic anhydride and the hydroxyl group.

In some embodiments, the biomolecule is attached to the acrylic linker via a Michael addition reaction between a nucleophilic group in the biomolecule and an alkenyl group in the acrylic linker.

In some embodiments, the nucleophilic group comprises a thiol group.

In some embodiments, the biomolecule comprises a protein or a peptide.

In some embodiments, the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

In some embodiments, the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

In some embodiments, the concentration of the biomolecule in the hydrogel ranges from about 0.05 mM to about 10 mM.

In some embodiments, the storage modulus of the hydrogel at 37° C. or 25° C. ranges from about 500 Pa to about 25,000 Pa.

In some embodiments, the elastic modulus of the hydrogel at 37° C. or 25° C. ranges from about 1 kPa to about 75 kPa.

In some embodiments, the storage modulus of the hydrogel is higher than the loss modulus of the hydrogel at 37° C. or 25° C.

In some embodiments, one of the following applies: (a) the hydrogel does not comprise a cell, (b) the hydrogel comprises a cell, optionally a stem cell, optionally a mesenchymal stem cell (MSC).

In some aspects, the present invention is directed to a hyaluronic acid (HA)-based hydrogel.

In some embodiments, the HA-based hydrogel comprises: a first HA polymer comprising a first crosslinker; a second HA polymer comprising a second crosslinker; a biomolecule attached to the first HA polymer or the second HA polymer through an acrylic linker attached to a hydroxyl group in the first HA polymer or the second HA polymer; and water.

In some embodiments, the sum of the amount of the first polymer and the amount of the second polymer in the HA-based hydrogel ranges from about 1% w/v to about 10% w/v based on the total volume of the HA-based hydrogel.

In some embodiments, the first crosslinker and the second crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

In some embodiments, the molar % of repeating units in the first HA polymer and the second HA polymer that are directly attached to the crosslinker ranges from about 10% to about 60%.

In some embodiments, the acrylic linker is attached to the hydroxyl group in the first HA polymer or the second HA polymer via an esterification reaction between an acrylic anhydride and the hydroxyl group.

In some embodiments, the biomolecule is attached to the acrylic linker via a Michael addition reaction between a nucleophilic group in the biomolecule and an alkenyl group in the acrylic linker.

In some embodiments, the nucleophilic group comprises a thiol group.

In some embodiments, the biomolecule comprises a protein or a peptide.

In some embodiments, the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

In some embodiments, the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

In some embodiments, the concentration of the biomolecule in the HA-based hydrogel ranges from about 0.05 mM to about 10 mM.

In some embodiments, the storage modulus of the HA-based hydrogel at 37° C. or 25° C. ranges from about 500 Pa to about 25,000 Pa.

In some embodiments, the elastic modulus of the HA-based hydrogel at 37° C. or 25° C. ranges from about 1 kPa to about 75 kPa.

In some embodiments, the storage modulus of the HA-based hydrogel is higher than the loss modulus of the HA-based hydrogel at 37° C. or 25° C.

In some embodiments, one of the following applies: (a) the HA-based hydrogel does not comprise a cell, (b) the HA-based hydrogel comprises a cell, optionally a stem cell, optionally a mesenchymal stem cell (MSC).

In some aspects, the present invention is directed to a method of preparing a HA-based hydrogel.

In some embodiments, the method comprises: attaching a first crosslinker to a first HA polymer; attaching a second crosslinker to a second HA polymer; attaching an acrylic linker to a hydroxyl group of the first HA polymer or the second HA polymer; attaching a biomolecule to the acrylic link; mixing the first HA polymer attached with the first crosslinker, the second HA polymer attached with the second crosslinker and water; crosslinking the first crosslinker and the second crosslinker.

In some embodiments, the sum of the amount of the first polymer and the amount of the second polymer in the HA-based hydrogel ranges from about 1% w/v to about 10% w/v based on the total volume of the mixture of the first HA polymer, the second HA polymer, and the water.

In some embodiments, the first crosslinker and the second crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

In some embodiments, the molar percentage of repeating units in the first HA polymer and the second HA polymer that are directly attached to the crosslinker ranges from about 10% to about 60%.

In some embodiments, attaching the acrylic linker to the hydroxyl group of the first HA polymer or the second HA polymer comprises attaching the acrylic linker to the hydroxyl group via an esterification reaction between an acrylic anhydride and the hydroxyl group.

In some embodiments, attaching the biomolecule to the acrylic linker comprises attaching a nucleophilic group the biomolecule to an alkenyl group of the acrylic linker via a Michael addition reaction.

In some embodiments, the nucleophilic group comprises a thiol group.

In some embodiments, the biomolecule comprises a protein or a peptide.

In some embodiments, the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

In some embodiments, the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

In some embodiments, the concentration of the biomolecule in the mixture of the first HA polymer, the second HA polymer and the water ranges from about 0.05 mM to about 10 mM.

In some embodiments, the storage modulus of the HA-based hydrogel at 37° C. or 25° C. ranges from about 500 Pa to about 25,000 Pa.

In some embodiments, the elastic modulus of the HA-based hydrogel at 37° C. or 25° C. ranges from about 1 kPa to about 75 kPa.

In some embodiments, the storage modulus of the HA-based hydrogel is higher than the loss modulus of the HA-based hydrogel at 37° C. or 25° C.

In some embodiments, one of the following applies: (a) the mixture of the first HA polymer, the second HA polymer and the water does not comprise a cell, (b) the mixture of the first HA polymer, the second HA polymer and the water comprises a cell, such as a stem cell.

In some aspects, the present study is directed to a method of promoting regeneration of a tissue in a subject in need thereof.

In some embodiments, the method comprises: placing a hydrogel or a HA-based hydrogel at a site in need of regeneration in the subject. In some embodiments, the hydrogel or HA-based hydrogel is the same as or similar to those as described elsewhere herein, such as in this section.

In some embodiments, placing the hydrogel or the HA-based hydrogel at the site comprises forming the hydrogel or the HA-based hydrogel at the site.

In some embodiments, forming the hydrogel or the HA-based hydrogel at the site comprises injecting a liquid mixture for forming the hydrogel or the HA-based hydrogel into the site.

In some embodiments, the mixture undergoes crosslinking and gelates (gels) to form the hydrogel or the HA-based hydrogel spontaneously at the site.

In some embodiments, the tissue is a bone tissue, a cartilage tissue, or combinations thereof.

In some embodiments, the biomolecule attached to the hydrogel polymer, the first HA polymer or the second HA polymer is bone morphogenetic protein 2 (BMP-2), or a functional mimicking peptide of BMP-2.

In some embodiments, the biomolecule is the functional mimicking peptide of BMP-2, and wherein the functional mimicking peptide of BMP-2 comprises at least one of the following amino acid sequences: GCGGGDWIVAG (SEQ ID NO:8), NSVNSKIPKACCVPTELSAI (SEQ ID NO: 9), or KIPKASSVPTELSAISTLYL (SEQ ID NO:10).

In some embodiments, the hydrogel or the HA-based hydrogel is placed in a femur of the subject.

In some embodiments, the subject is suffering from osteoporosis.

In some embodiments, the subject is a mammal, optionally a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1G illustrate certain aspects of macromer synthesis and HA Nor-Tet hydrogel polymerization, in accordance with some embodiments. FIG. 1A: HA macromers modified with Nor (HANor) or (FIG. 1B) Tet (HATet) (FIG. 1C) spontaneously from stable crosslinked hydrogels when combined. FIG. 1D: Representative time sweep rheology plot of 2% w/v HA Nor-Tet hydrogels polymerizing at 37° C. for 60 mins and 180 mins. FIG. 1E: HANor and HATet macromers can be dissolved rapidly at room temperature and loaded into syringes for mixing through a Luer-Lock coupler. Mixed macromers remain in solution long enough for extrusion into (FIG. 1F) molds or (FIG. 1G) cavities, resulting in self-forming hydrogels that conform to the shape of space they occupy.

FIGS. 2A-2H illustrates certain aspects of the physical characterization of self-forming hydrogels at 37° C., in accordance with some embodiments. FIG. 2A: Representative frequency sweep rheology plot shows storage (G′) and loss moduli (G″) of 2%, 4%, and 6% w/v HA Nor-Tet hydrogels at a 1:1 stoichiometric ratio. FIG. 2B: Representative time sweep rheology plot was used to determine (FIG. 2C) plateau G′ and (FIG. 2D) time to 50% plateau G′ of 2%, 4%, and 6% w/v HA Nor-Tet hydrogels at a 1:1 stoichiometric ratio. FIG. 2E: HANor and HATet were mixed at a 1:2, 1:1, or 2:1 stoichiometric ratio and the plateau G′ was determined for (FIG. 2F) 2%, (FIG. 2G) 4%, and (FIG. 2H) 6% w/v HA Nor-Tet hydrogels. Bar graphs shown as mean±SD (n≥3 samples per condition) with significant differences determined with ANOVA followed by Tukey's post hoc test where ***p<0.005.

FIGS. 3A-3C illustrate certain aspects of HANor macromers functionalized with thiolated peptides, in accordance with some embodiments. FIG. 3A: Hydroxyl groups in HANor macromers are coupled with methacrylic anhydride to form HANor Me macromers. FIG. 3B HANor Me macromers are then mixed with thiolated RGD adhesive peptide (cRGD) to form peptide functionalized HANor (cRGD+) macromers. FIG. 3C: 1H NMR spectra shows peaks corresponding to norbornene (left), methacrylate (middle), and cRGD (right) modifications to the HA backbone.

FIGS. 4A-4D demonstrate that peptide coupling does not affect mechanical properties of HA Nor-Tet hydrogels, in accordance with some embodiments. FIG. 4A: Representative time sweep rheology of G′ kinetics for 2%, 4%, and 6% w/v HA Nor-Tet hydrogels was used to determine (FIG. 4B) plateau G′ and (FIG. 4C) time to 50% plateau G′. FIG. 4D: Compression testing of 2%, 4%, and 6% w/v HA Nor-Tet hydrogels formed in cylindrical molds was used to determine elastic modulus (E). Bar graphs shown as mean±SD (n≥3 samples per condition) with significant differences determined with ANOVA followed by Tukey's post hoc test where ***p<0.005.

FIGS. 5A-5E illustrate certain aspects of 2D cell attachment and proliferation on cRGD− functionalized HA Nor-Tet hydrogels, in accordance with some embodiments. FIG. 5A: HANor macromers with or without cRGD functionalization were mixed with HATet, injected into cylindrical molds, and (FIG. 5B) human MSCs were cultured on cRGD+ and cRGD− HA Nor-Tet hydrogels. FIG. 5C: Representative maximum projection images (actin, red; nuclei, blue) of MSCs on Soft or Stiff HA Nor-Tet hydrogels with or without covalently bound cRGD. Confocal images were used to determine MSC Density (number of nuclei cm⁻²) on (FIG. 5D) Soft and Stiff HA Nor-Tet hydrogels with or without cRGD. AlamarBlue assay was performed on day 1, 3, and 7 to quantify Normalized metabolic activity of MSCs on RGD-functionalized (FIG. 5E) Soft and Stiff HA Nor-Tet hydrogels. Bar graphs shown as mean±SD (n≥3 samples per condition) with significant differences determined with ANOVA followed by Tukey's post hoc test where ***p<0.005. Scale bar: 500 μm.

FIGS. 6A-6D demonstrate that cells on 2D HA Nor-Tet hydrogels display stiffness-dependent changes in cell morphology, in accordance with some embodiments. FIG. 6A: Representative silhouettes of MSCs on Soft and Stiff HA Nor-Tet hydrogels. Bar graphs show (FIG. 6B) Area, (FIG. 6C) Circularity, and (FIG. 6D) Aspect ratio of MSCs on Soft and Stiff HA Nor-Tet hydrogels. Bar graphs shown as mean±SD (n≥3 samples per condition) with significant differences determined with ANOVA followed by Tukey's post hoc test where ***p<0.005. Scale bar: 100 μm.

FIG. 7A-7C demonstrate that MSCs are mechanosensitive on 2D Soft and Stiff HA Nor-Tet hydrogels, in accordance with some embodiments. FIG. 7A: Representative images and Nuclear YAP (green) quantification of MSCs on Soft and Stiff HA Nor-Tet hydrogels. FIG. 7B: Representative images of MSCs stained for pFAK (green), phalloidin (red), and nuclei (blue) and quantification of pFAK maturation (length, average number of adhesions per cell) of MSCs on Soft and Stiff HA Nor-Tet hydrogels. Bar graphs shown as mean±SD (n≥3 samples per condition) with significant differences determined with ANOVA followed by Tukey's post hoc test where ***p<0.005. Scale bars: a, b, c 50 μm.

FIGS. 8A-8E demonstrate that MSCs in injectable 3D HA Nor-Tet hydrogels are highly viable, in accordance with some embodiments. FIG. 8A: HA Nor-Tet hydrogel solutions consisting of MSCs mixed with RGD-functionalized HANor and HATet were injected into cylindrical molds via extrusion of various clinically relevant needle sizes. Representative live (green) and dead (red) staining of MSCs cultured in (FIG. 8B) Soft and (FIG. 8C) Stiff HA Nor-Tet hydrogels. Bar graphs show percentage of live cells in (FIG. 8D) Soft and (FIG. 8E) Stiff HA Nor-Tet hydrogels from confocal image analysis. Bar graphs shown as mean±SD (n≥3 samples per condition). Scale bar: 500 μm.

FIG. 9 illustrates certain aspects of storage modulus (G′) evolution through time sweep rheometry for 2% w/v HA Nor-Tet hydrogels normalized to plateau G′ of the HANor+HATet group (black diamond), in accordance with some embodiments. Mixing of HANor with unmodified HA (blue diamond) or HATet with unmodified HA (red diamond) does not result in crosslinking.

FIG. 10 illustrates certain aspects of storage modulus (G′) evolution through time sweep rheometry for 2% w/v HA Nor-Tet hydrogels normalized to plateau G′ of the HANor+HATet group (black diamond), in accordance with some embodiments. Addition of ˜1,000× free Nor (blue diamond) or Tet (red diamond) small molecules to the mixture results in inhibition of crosslinking. 1,000× molar excess of free Nor is equivalent to 2 mM free Nor or 2.5 mM Nor-NH₂. 1,000× molar excess of free Tet is equivalent to 1.4 mM free Tet or 1.6 mM Tet-NH₂.

FIGS. 11A-11B are 1H NMR spectra of HA modified with (FIG. 11A) Nor or (FIG. 11B) Tet moieties, in accordance with some embodiments.

FIG. 12 depicts elastic moduli of 2%, 4%, and 6% w/v HA Nor-Tet hydrogels, in accordance with some embodiments. Values were determined using compression mechanical testing. Scatter dot plot shown as mean±SD (n≥6 samples per condition) with significant differences determined with ANOVA followed by Tukey's post hoc test where ***p<0.001.

FIGS. 13A-13C illustrate certain aspects of the physical characterization of HA Nor-Tet hydrogels at 37° C., in accordance with some embodiments. Time to 50% plateau G′ of (FIG. 13A) 2%, (FIG. 13B) 4%, and (FIG. 13C) 6% w/v HA Nor-Tet hydrogels at 1:2, 1:1, and 2:1 HANor: HATet stoichiometric ratios. Bar graphs shown as mean±SD (n≥3 samples per condition).

FIGS. 14A-14C illustrate certain aspects of the physical characterization of HA Nor-Tet hydrogels at 37° C., in accordance with some embodiments. Plateau G′ of (FIG. 14A) 2%, (FIG. 14B) 4%, and (FIG. 14C) 6% w/v HA Nor-Tet hydrogels at 1:1, and 1:1.25 HANor: HATet stoichiometric ratios. Bar graphs shown as mean±SD (n≥3 samples per condition).

FIGS. 15A-15C are 1H NMR spectra of cRGD+ HANorMe macromers, in accordance with some embodiments. Distinct peaks for cRGD peptides are seen for HANor macromers for (FIG. 15A) 2%, (FIG. 15B) 4%, and (FIG. 15C) 6% w/v HA Nor-Tet hydrogels.

FIGS. 16A-16C illustrate certain aspects of 3D viability of MSCs injected through different size syringe needles, in accordance with some embodiments. FIG. 16A: Needles used for extrusion have internal diameters ranging from 0.16 to 1.19 mm. Representative live (green) and dead (red) staining of MSCs extruded through different needle sizes for (FIG. 16B) 2% and (FIG. 16C) 6% w/v HA Nor-Tet hydrogels. Scale bar: 500 μm.

FIGS. 17A-17C illustrate certain aspects of 3D morphology of MSCs encapsulated in 2% and 6% w/v HA Nor-Tet hydrogels, in accordance with some embodiments. FIG. 17A: Representative F-actin (red) and nuclear (blue) staining of an MSC encapsulated in HA Nor-Tet hydrogels (2% w/v shown). Quantification of (FIG. 17B) Volume and (FIG. 17C) sphericity of MSCs in 2% and 6% w/v HA Nor-Tet hydrogels. Bar graphs shown as mean±SD (n≥3 samples per condition) with no significant differences (ns) determined with ANOVA. Scale bar: 50 μm.

FIGS. 18A-18B illustrate certain aspects of 3D mechanosensing of MSCs encapsulated in 2% and 6% w/v HA Nor-Tet hydrogels, in accordance with some embodiments. FIG. 18A: Representative image of an MSC encapsulated in a 2% w/v HA Nor-Tet hydrogel stained for YAP (green). FIG. 18B: Nuclear YAP quantification of MSCs on Soft and Stiff Nor-Tet hydrogels stained for YAP. Bar graphs shown as mean±SD (n≥3 samples per condition) with no significant differences (ns) determined with ANOVA. Scale bar: 50 μm.

FIGS. 19A-19D illustrate certain aspects of in vitro degradation of self-forming HA Nor-Tet hydrogels, in accordance with some embodiments. FIG. 19A: Hyaluronidase cleaves glycosidic bonds in the HA backbone. Degradation kinetics of 2%, 4%, and 6% w/v HA Nor-Tet hydrogels at (FIG. 19B) 0, (FIG. 19C) 2.5, and (FIG. 19D) 25 U mL-1 concentrations of hyaluronidase. Without hyaluronidase, HA Nor-Tet hydrogels retain their dry weight for the duration of the study (230 h). At a hyaluronidase concentration of 2.5 U mL-1, Soft (2% w/v) hydrogels fully degrade within ˜40 h while Stiff (6% w/v) hydrogels fully degrade within ˜225 h. Soft and Stiff HA Nor-Tet hydrogels fully degrade within ˜10 h and ˜60 h, respectively when exposed to a hyaluronidase concentration of 25 U mL-1. Stiff hydrogels take approximately 5.5× the amount of time needed by Soft hydrogels to fully degrade when exposed to low (2.5 U mL⁻¹) or high (25 U mL⁻¹) hyaluronidase concentrations.

FIGS. 20A-20F demonstrate that the MSC spreading and differentiation can be controlled in 3D Nor-Tet hydrogels, in accordance with some embodiments. FIG. 20A: Quantification of MSC volume (left) and sphericity (right) of MSCs in stable or degradable Nor-Tet hydrogels after 1 and 7 days in culture. FIGS. 20B-20C: Representative images of MSCs stained for actin (red) and nuclei (blue) after 1 and 7 days in culture inside stable (FIG. 20B) or degradable (FIG. 20C) Nor-Tet hydrogels. FIG. 20D: Quantification of percentage of AD(+) and OS(+) cells in stable HA and degradable gel Nor-Tet hydrogels after 7 days in culture. FIGS. 20E-20F: Representative images of cells stained for ALP (magenta) and lipid droplets (green) in stable (FIG. 20F) or degradable (FIG. 20F) Nor-Tet hydrogels after 7 days in culture. Bar graphs shown as mean±SD (n≥3 samples per condition) with significant differences determined with ANOVA followed by Tukey's post hoc test where ***p<0.001 and ns is not significant. Scalebars: 50 μm (FIG. 20D) or 500 μm (FIG. 20F).

FIGS. 21A-21E illustrate certain aspects of the synthesis of HANorDWIVA (DWIVA, SEQ ID NO:34) and characterization of DWIVA-functionalized Nor-Tet hydrogels, in accordance with some embodiments. FIG. 21A: Methacrylic anhydride is coupled to the HANor macromer to provide sites for DWIVA (SEQ ID NO:34) to bind through Michael addition reaction. FIG. 21B: Time sweep rheology of G′ kinetics is used to determine (FIG. 21C) plateau G′ and (FIG. 21D) time to 50% plateau G′ of 0, 0.5, and 2.0 mM DWIVA-functionalized Nor-Tet hydrogels. FIG. 21E: Elastic moduli of hydrogels with 0, 0.5, and 2.0 mM DWIVA. Graphs and scatter dot plot are shown as mean±SD (n≥3 samples per condition) with no significant difference (ns) determined with one-way ANOVA.

FIGS. 22A-22E demonstrate that the soluble presentation of DWIVA (SEQ ID NO:34) enhances ALP levels of MSCs on glass, in accordance with some embodiments. FIG. 22A: ALP staining of MSCs (dark blue) on glass in Growth Medium (negative control), Growth Medium supplemented with 0.5 or 2.0 mM DWIVA, and OS Medium (positive control). FIG. 22B: Representative fluorescence staining of ALP (magenta) and nucleus (blue) of MSCs on glass in Growth Medium, Growth Medium supplemented with 0.5 or 2.0 mM DWIVA, and OS Medium.

FIG. 22C: Frequency distribution of ALP MFI of MSCs seeded on glass coverslips in Growth Medium (negative group) and OS Medium (positive group); dashed line shows MFI cutoff for ALP(−) and ALP(+) cells. FIG. 22D: Representative cells that are ALP(−) (top, MFI<52) and ALP(+) (bottom, MFI≥294 52). FIG. 22F: % ALP(+) quantification of MSCs cultured in Growth Medium with 0, 0.5, or 2.0 mM soluble DWIVA peptide and OS Medium. Bar graphs are shown as mean±SD (n≥50 cells per condition) with nonsignificant differences denoted as ns, and significant differences determined with ANOVA followed by Tukey's post hoc test where **p<0.01, ***p<0.001. Scale bars: (FIG. 22A) 2 mm, (FIG. 22B) 100 μm, (FIG. 22D) 20 μm.

FIGS. 23A-23D demonstrate that the immobilized DWIVA (SEQ ID NO:34) enhances ALP levels of MSCs on 2D Nor-Tet hydrogels, in accordance with some embodiments. FIG. 23A: HANor functionalized with RGD (2 mM) and DWIVA (0, 0.5, or 2 mM) were mixed with HATet, injected into cylindrical molds, and MSCs seeded atop. FIG. 23B: Representative ALP (magenta) and nucleus (blue) staining of MSCs seeded on Nor-Tet hydrogels with 0, 0.5, or 2 mM DWIVA functionalization and OS Medium positive control. FIG. 23C: Frequency distribution of ALP MFI of MSCs seeded on Nor-Tet hydrogels cultured in Growth Medium (negative group) or OS Medium (positive group); dashed line shows MFI cutoff for ALP(−) and ALP(+) cells. FIG. 23D: % ALP(+) quantification of cultured on Nor-Tet hydrogels in Growth Medium with 0, 0.5, or 2.0 mM immobilized DWIVA. Bar graphs are shown as mean±SD, (n≥50 cells per condition) with nonsignificant differences denoted as ns, and significant differences determined with ANOVA followed by Tukey's post hoc test where **p<0.01, ***p<0.001. Scale bar: (FIG. 23B) 20 μm.

FIGS. 24A-24F demonstrate that immobilized DWIVA (SEQ ID NO:34) enhances ALP levels of MSCs in 3D Nor-Tet hydrogels, in accordance with some embodiments. FIG. 24A: MSCs were resuspended in RGD-functionalized HANor with or without DWIVA coupling, mixed with HATet, and injected into cylindrical molds. Representative ALP staining of MSCs (dark blue) in 3D Nor-Tet hydrogels with (FIG. 24B) 0 mM DWIVA and Growth Medium or OS Medium and (FIG. 24C) Growth Medium with 0.5 mM or 2.0 mM DWIVA coupling. (FIG. 24D) Frequency distribution of ALP MFI of encapsulated in 3D Nor-Tet hydrogels (0 mM DWIVA) cultured in Growth Medium (negative group) or OS Medium (positive group); dashed line shows MFI cutoff for ALP(−) and ALP(+) cells. FIG. 24E: % ALP(+) quantification of MSCs cultured in 3D Nor-Tet hydrogels functionalized with 0, 0.5, or 2.0 mM immobilized DWIVA. FIG. 24F: Representative images of single MSCs (ALP, magenta; nuclei, blue) in Growth Medium with 0 mM or 2.0 mM DWIVA coupling. Bar graphs are shown as mean±SD, (n≥50 cells per condition) with nonsignificant differences denoted as ns, and significant differences determined with ANOVA followed by Tukey's post hoc test where *p<0.05, **p<0.01, ***p<0.001. Scale bars: (FIGS. 24B-24C) 2 mm, (FIG. 24F) 20 μm.

FIGS. 25A-25E demonstrate that DWIVA (SEQ ID NO:34)-functionalized hydrogels induce trabecular bone growth in vivo, in accordance with some embodiments. FIG. 25A: The knee joint is exposed, and a drill is used to clear the femoral canal. FIG. 25B: The left femur is injected with Nor-Tet hydrogels (Gel group) and the right femur is injected with DWIVA-containing Nor-Tet hydrogels (Peptide group). FIG. 25C: Schematic shows 3D regions of interest that were imaged using micro-CT (blue, coronal view of distal shaft; orange rectangle, axial view of distal shaft center). Representative micro-CT scans of (FIG. 25D) the distal shaft and (FIG. 25F) the distal shaft center of drilled femurs, and drilled femurs injected with Gel and Peptide. Scale bars: (FIGS. 25D-25E) 500 μm.

FIGS. 26A-26B: 1H-NMR characterization of HANor-cDWIVA, in accordance with some embodiments. Proton peaks for Nor are between δ 6.2 to 6.3 ppm and proton peaks for Me are between δ 5.5 to 6.5 ppm. FIG. 26A: 0.50 mM DWIVA and FIG. 26B: 2.0 mM DWIVA peaks are between δ 0.50 to 1.50 ppm.

FIGS. 27A-27D: MFI determination of cytoplasmic ALP signal for single MSCs, in accordance with some embodiments. FIG. 27A: Representative image of two MSCs stained for ALP (magenta) and nucleus (blue and white dashed outline). FIG. 27B: The nucleus channel is subject to an Otsu-based threshold to create a binary mask. FIG. 27C: The nuclei masks are dilated, inverted, and overlaid with the original nuclear mask to create ring-shaped regions of interest (ROIs). FIG. 27D: The ring-shaped ROIs are applied to the ALP channel and the 3D Objects Counter is applied to calculate MFI, which is the integrated density of ALP signal divided by the ring-shaped ROI area. Scale bar: 50 μm.

FIG. 28 demonstrates that MSCs in Nor-Tet hydrogels are highly viable, in accordance with some embodiments. Nor-Tet hydrogel solutions consisting of MSCs mixed with RGD-functionalized HANor with different concentrations of DWIVA (0, 0.5, or 2.0 mM) and HATet were injected into cylindrical molds via extrusion through a 16 G needle. Bar graph shows percentage of live cells after 1 day and 7 days from confocal images. Bar graphs shown as mean±SD (n≥3 samples per condition).

FIG. 29: Reaction parameters to create DWIVA-functionalized HANorMe macromers that when mixed with HATet form 2 wt % Nor-Tet hydrogels with 0.5 or 2.0 mM effective DWIVA (SEQ ID NO:34) concentrations, in accordance with some embodiments.

FIGS. 30A-30C demonstrate that DWIVA-functionalized hydrogels induce trabecular bone growth in vivo, in accordance with some embodiments. FIG. 30A: the sites of injection and region of interest for CT imaging. FIG. 30B: comparison of drilled femurs viewed through axial slices not treated with hydrogel (“CD”), treated with hydrogels not functionalized with DWIVA peptide (SEQ ID NO:34) and without mesenchymal stem cells (“CG”), treated with hydrogels functionalized with DWIVA peptide but without mesenchymal stem cell (“EB”), or treated with hydrogels functionalized with DWIVA peptide and includes mesenchymal stem cells (“EBC”) at two weeks and four weeks. FIG. 30C: quantification for FIG. 30B, volume of new trabecular bone growth versus volume of region analyzed (“Bone fraction”), number of trabecular bone connections (“Count”), thickness of trabecular bone (“Thickness”) and distance between each trabecular bone (“Spacing”).

FIGS. 31A-31D: Injection of hydrogel-cell composition in the femur of mice, in accordance with some embodiments. FIGS. 31A-31B: In each test animal, the left femur was used as a control, and the right femur was injected with a hydrogel solution. FIG. 31C: micro-CT scanning photos of a representative femur injected with the hydrogel. FIG. 31D: region of the micro-CT scanning.

FIGS. 32A-32E demonstrate that the injection of DWIVA peptide (SEQ ID NO:34)-functionalized hydrogels in femur result in more robust bone regeneration as compared to non-functionalized hydrogels, in accordance with some embodiments. FIG. 32A: Trabeculae morphometry image (coronal views) of femurs injected with DWIVA peptide-functionalized hydrogel (P group) and hydrogels without peptides (C group), at two- and four-weeks post-injection. By week four, the peptide-functionalized P group had a significant growth advantage over the non-functionalized hydrogel C group. FIGS. 32B-32E: Measurement of trabeculae bone volume (FIG. 32B), number (FIG. 32C), thickness (FIG. 32D) and spacing (FIG. 32E). While no significant difference in volume was found between the groups at week two, a substantial increase in trabeculae volume was observed by the fourth week (FIG. 32B). The number of trabeculae was higher in the P group at both time intervals, with a more significant difference observed between the two groups at week two compared to week four (FIG. 32C). The thickness of trabecular was also greater in the peptide-functionalized P group at weeks two and four than in the non-functionalized hydrogel C group (FIG. 32D). Additionally, the peptide-functionalized P group exhibited further support for enhanced bone growth over time, as evidenced by a significantly lower trabecular spacing, particularly at week four (FIG. 32E).

FIGS. 33A-33E demonstrate that the injection of MSC-laden DWIVA peptide (SEQ ID NO: 34)-functionalized hydrogels in femur result in more robust bone regeneration as compared to DWIVA peptide-functionalized hydrogels without MSC cells, in accordance with some embodiments. FIG. 33A: Coronal views of treated femurs showed more trabecular growth in the PC group (MSC-laden peptide functionalized hydrogel group) when compared to the P group (peptide functionalized hydrogel group without MSC), with a significant increase in bone growth observed at four weeks post-injection. FIGS. 33B-33F: Measurement of trabeculae bone volume (FIG. 33B), number (FIG. 33C), thickness (FIG. 33D) and spacing (FIG. 33E). FIG. 33B: There were significant increases reported in trabeculae volume, number, and thickness, as well as decreased trabeculae spacing from weeks two to four. Though the trabeculae volume of the peptide functionalized hydrogel only P group wasn't as high as that of the PC group, it did still increase over time. The number (FIG. 33C) and thickness (FIG. 33D) of trabeculae in the treated femur increased as a function of encapsulated MSCs, especially after four weeks. More significance is calculated for trabeculae number when compared to trabeculae thickness between both groups at both time intervals. FIG. 33E: the trabeculae spacing of the P and PC groups was significantly different at two weeks, however, there was not a significant difference at four weeks.

FIGS. 34A-34D: histologically evaluation of bone growth in groups C, P and PC, in accordance with some embodiments. To histologically evaluate bone growth in groups C, P, and PC, H&E and Masson's Trichrome staining techniques were used on tissue sections of the extracted rat femurs. ALP and TRAP stains were then performed for the determination of osteoblast and osteoclast formations. Cortical bone (labelled as CB) exists as the outer layer that surrounds and supports the trabecular bone. Enlarged images of the PC group include arrows which indicate regions of trabecular bone growth.

FIGS. 35A-35D: histologically evaluation of bone growth in groups C, P and PC, in accordance with some embodiments. To histologically evaluate bone growth in groups C, P, and PC, H&E and Masson's Trichrome staining techniques were used on tissue sections of the extracted rat femurs. ALP and TRAP stains were then performed for the determination of osteoblast and osteoclast formations. Cortical bone (labelled as CB) exists as the outer layer that surrounds and supports the trabecular bone. Enlarged images of the PC group include asterisks which represent osteoblasts (brown) and osteoclast (orange) formations.

FIGS. 36A-36B: Quantitative analysis of the number of osteoblast (FIG. 36A) and osteoclast (FIG. 36B) formation per unit area at four weeks post-injection for the C group (control group, hydrogels without peptide functionalization), P group (DWIVA peptide-functionalized hydrogels) and PC group (MSC-laden DWIVA peptide-functionalized hydrogels), in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In the study described herein, an injectable hydrogel suitable for promoting local tissue regeneration was developed.

The hydrogels developed in the present study include molecules, such as peptides, attached to the polymer network via the hydroxyl groups in the network. The attached molecules allow desirable hydrogel-cell interaction needed for the local tissue regeneration to be achieved.

The hydrogels developed in the present study also have highly tunable gelation times and mechanical properties. Since such parameters also affect the interaction between the hydrogel and cells, as well as the behavior of cells on or inside the hydrogel, such highly tunable nature of the hydrogels is desirable, as well.

Using mice trabecular bone regeneration as a non-limiting model, the present study demonstrated that several illustrative hydrogels developed in the present study were able to promote the regeneration of damaged tissues when placed at the site where regeneration was needed.

Accordingly, in some aspects, the present invention is directed to a hydrogel.

In some aspects, the present invention is directed to a method of preparing a hydrogel.

In some aspects, the present invention is directed to a method of promoting tissue regeneration in a subject in need thereof.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

### Hydrogel

In some aspects, the instant specification is directed to a hydrogel.

In some embodiments, the hydrogel includes a hydrogel polymer. In some embodiments, the hydrogel includes a crosslinker crosslinking the hydrogel polymer. In some embodiments, the hydrogel includes a biomolecule attached to the hydrogel polymer. In some embodiments, the hydrogel includes water.

In some embodiments, the biomolecule is attached to the hydrogel polymer through an acrylic linker, which is attached to a hydroxyl group in the hydrogel polymer.

In some embodiments, the hydrogel polymer is any polymer that is suitable for forming hydrogel, as long as the polymer has a hydroxyl group in the repeating unit for the acrylic linker to attach. Non-limiting examples of the polymers include an alginate polymer, an α,β-poly(N-hydroxyethyl)-DL-aspartamide polymer, a chitosan polymer, a chondroitin sulfate polymer, a collagen/gelatin polymer, an elastin polymer, a fibrin polymer, a heparin polymer, a hyaluronic acid polymer, and a poly(vinyl alcohol) polymer, and the like. One of ordinary skill in the art would understand that alginate polymers, α,β-poly(N-hydroxyethyl)-DL-aspartamide polymers, chitosan polymers, chondroitin sulfate polymers, heparin polymers, hyaluronic acid polymers, and poly(vinyl alcohol) polymers naturally include hydroxyl groups in the repeating units. Collagen/gelatin polymers, elastin polymers, and fibrin polymers are proteins which either naturally include serine and/or threonine residues or can be modified to include serine/threonine residues, which include hydroxyl groups on the side chains thereof.

In some embodiments, the amount of the hydrogel polymer in the hydrogel ranges from about 1% w/v to about 10% w/v, such as from about 1% w/v to about 2% w/v, from about 1.5% w/v to about 2.5% w/v, from about 2% w/v to about 3% w/v, from about 2.5% w/v to about 3.5% w/v, from about 3% w/v to about 4% w/v, from about 3.5% w/v to about 4.5% w/v, from about 4% w/v to about 5% w/v, from about 4.5% w/v to about 5.5% w/v, from about 5% w/v to about 6% w/v, from about 5.5% w/v to about 6.5% w/v, from about 6% w/v to about 7% w/v, from about 6.5% w/v to about 7.5% w/v, from about 7% w/v to about 8% w/v, from about 7.5% w/v to about 8.5% w/v, from about 8% w/v to about 9% w/v, from about 8.5% w/v to about 9.5% w/v, or from about 9% w/v to about 10% w/v, based on a total volume of the hydrogel. In some embodiments, the amount of the hydrogel polymer in the hydrogel is about 1% w/v, about 2% w/v, about 3% w/v, about 4% w/v, about 5% w/v, about 6% w/v, about 7% w/v, about 8% w/v, about 9% w/v, about 10% w/v, or any ranges therebetween, based on the total volume of the hydrogel. As described elsewhere herein, in general, the stiffness of the hydrogel herein increases as the amount of the hydrogel polymer in the hydrogel increases. Also as described herein, the stiffness of the hydrogel affects, among others, the survival, proliferation, morphology, protein expression, differentiation, tissue formation of cells on or inside the hydrogel, and can be adjusted according to the specific application.

In some embodiments, one of more of the hydrogel polymers forming the hydrogel is degradable under physiological conditions or by a cell embedded in the hydrogel. For example, when hyaluronidase is present in the physiological condition or the cells embedded in the hydrogel express hyaluronidase, the hydrogel polymers can include hyaluronic acid. When collagen digesting enzymes such as collagenases are present in the physiological condition or the cells embedded in the hydrogel express such enzymes, the hydrogel polymers can include gelatin.

One of ordinary skill in the art would understand that hydrogels can be crosslinked by various methods.

For example, the polymers can be crosslinked by small-molecule cross-linking, such as by bi-functional molecule (or multi-functional molecule) crosslinkers that interconnect the polymer chains. Non-limiting examples of small-molecule crosslinking include those involving aldehyde and amino groups to form Schiff base. Such crosslinkers include dialdehydes such as glyoxal and glutaraldehyde (which that forms covalent imine bonds with the amino groups, such as amino groups of chitosan, via Schiff reaction). Horseradish peroxidase (HRP) and hydrogen peroxide (H₂O₂) can be used as cross-linkers to crosslink, for example, polymers including tyramine groups. Genipin can be used to crosslink polymers with amino-terminated groups.

For example, the polymers can comprise, or be functionalized with, reactive functional groups, which eliminates the need for a separate crosslinker molecule. In some embodiments, the reactive functional groups are a nucleophile (e.g., an amine group or a thiol group) and a vinyl group, with can undergo a Michael addition to interconnect the polymer chains. In some embodiments, the reactive functional groups are an aldehyde group and a hydrazide group, which can form a hydrazone bond.

For example, in one embodiment, the polymers can comprise, or be functionalized with, a photo cross-linking group. In some embodiments, the photo cross-linking group is an azide group (—N₃), which is converted to nitrene group (R—N:) under the exposure to UV-light and binds to a free amino group. In some embodiments, the photo cross-linking group is an acrylate group (CH₂═CHCOO⁻), which crosslink to other acrylate group when irradiated by UV.

For example, in other embodiments, the polymers can be crosslinked by enzyme-catalyzed cross-linking reaction between the polymer chains. A number of enzymes including transglutaminases (TG), peroxidases, tyrosinase, phosphopantetheinyl transferase, lysyl oxidase, plasma amine oxidase, and phosphatases have been used to form the crosslinking. TG catalyzes the reaction between a free amine group of a protein or peptide-bound lysine and the g-carboxamide group of a protein or peptide-bound glutamine. HRP and soy bean peroxidase are examples of peroxidase enzymes that can catalyze the conjugation of phenol and aniline derivatives in the presence of substrate H₂O₂. Tyrosinases are oxidative enzymes that convert tyrosine residues (e.g. gelatin) into reactive o-quinone moiety, which can react with available nucleophiles such amino groups.

In some embodiments, the crosslinker includes a pair of a diene and a dienophile that undergoes a catalyst-free Diels-Alder reaction. Non-limiting examples of diene-dienophile pairs that undergo a catalyst-free Diels-Alder reaction include a furan-dimaleimide pair, a tetrazine-di-norbornene pair, a tetrazine-norbornene pair, and the like.

In some embodiments, the molar percentage of repeating units in the hydrogel polymer that are directly attached to the crosslinker ranges from about 10% to about 60%, such as from about 10% to about 15%, from about 15% to about 20%, from about 20% to about 25%, from about 25% to about 30%, from about 30% to about 35%, from about 35% to about 40%, from about 45% to about 50%, from about 50% to about 55%, or from about 55% to about 60%. In some embodiments, the molar percentage of repeating units in the hydrogel polymer that are directly attached to the crosslinker is about 10%, about 15%, about 20%, about 25%, about 30%, about 35%, about 40%, about 45%, about 50%, about 55%, about 60%, or any ranges therebetween. As described elsewhere herein, the degree of crosslinking affects the stiffness of the hydrogel, which affects many aspects of cells in contact with the hydrogel, such as survival, proliferation, morphology, protein expression, differentiation, tissue formation, and the like.

In some embodiments, the acrylic linker is a linker derived from the molecule

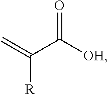

in which R is independently at each instance H, C₁-C₆ alkoxy, C₁-C₆ alkyl, C₁-C₆ alkenyl, and C₁-C₆ alkynyl, or C₃-C₆ cycloalkyl.

In some embodiments, the acrylic linker is attached to the hydroxyl group in the hydrogel polymer via an esterification reaction between a methacrylic anhydride and the hydroxyl group.

In some embodiments, the biomolecule is attached to the acrylic linker via a Michael addition reaction between a nucleophilic group in the biomolecule and the alkenyl group in the acrylic linker.

In some embodiments, the nucleophilic group comprises a thiol group.

In some embodiments, the biomolecule includes a protein or a peptide, or the like.

In some embodiments, the biomolecule includes an extracellular matrix protein or a functional mimicking peptide thereof, a growth factor or a functional mimicking peptide thereof, an angiogenic protein or a functional mimicking peptide thereof, a glycosaminoglycan (GAG)-binding peptide, or the like.

Examples of extracellular matrix proteins includes fibronectin, vitronectin, laminin α-chain, laminin β-chain, collagen type I, osteopontin, and the like. Examples of functional mimicking peptides of extracellular proteins include peptides comprising the amino acid sequence RGDS (SEQ ID NO: 1, functional mimicking peptide of fibronectin and vitronectin), peptides comprising the amino acid sequence YIGSR (SEQ ID NO:2, functional mimicking peptide of laminin α-chain), peptides comprising the amino acid sequence IKVAV (SEQ ID NO: 3, functional mimicking peptide of laminin β-chain), peptides comprising the amino acid sequence GFOGER (SEQ ID NO:4, functional mimicking peptide of collagen type I), peptides comprising the amino acid sequence DVDVPDGRGDSLAYG (SEQ ID NO:5, functional mimicking peptide of osteopontin), and the like.

Examples of growth factors include vascular endothelial growth factor (VEGF), osteogenic growth peptide (OGP), bone morphogenetic protein-2 (BMP-2), and the like. Examples of functional mimicking peptides of growth factors includes peptides comprising the amino acid sequence KLTWQELYQLKYKGI (SEQ ID NO:6, functional mimicking peptide of VEGF), peptides comprising the amino acid sequence YGFGG (SEQ ID NO:7, functional mimicking peptide of OGP), peptides comprising the amino acid sequence DWIVA (SEQ ID NO: 34), GCGGGDWIVAG (SEQ ID NO:8), NSVNSKIPKACCVPTELSAI (SEQ ID NO:9), or KIPKASSVPTELSAISTLYL (SEQ ID NO:10) (all four are functional mimicking peptides of BMP-2), and the like.

Examples of angiogenic proteins include VEGF, cellular communication network factor 1 (CCN1), secreted protein acidic and rich in cysteine (SPARC), thymosin beta-4, and the like. Examples of functional mimicking peptides includes peptides comprising the amino acid sequence KLTWQELYQLKYKGI (SEQ ID NO:6, functional mimicking peptide of VEGF), peptides comprising the amino acid sequence NCKHQCTCIDGAVGCIPLCP (SEQ ID NO:11, functional mimicking peptide of CCN1), peptides comprising the amino acid sequences TLEGTKKGHKLHLDY (SEQ ID NO:12) and KKGHK (SEQ ID NO:13) (both are functional mimicking peptides of SPARC), peptides comprising the amino acid sequence SDKP (SEQ ID NO: 14, functional mimicking peptide of thymosin beta-4).

Examples of GAG-binding peptides including peptides comprising the amino acid sequences PNDKKK (SEQ ID NO: 15), PNDRRR (SEQ ID NO:16) or GRPGKRGKQGQK (SEQ ID NO:17) (all are heparin-binding peptides), peptides comprising the amino acid sequences RYPISRPRKR (SEQ ID NO:18), CGGGRYPISRPRKR (SEQ ID NO:19), RYPISRPRKRC (SEQ ID NO:20) (all are hyaluronic acid-binding peptides), peptides comprising the amino acid sequence CGGGYKTNFRRYYRF (SEQ ID NO: 21, chondroitin sulfate binding peptide).

In some embodiments, the biomolecule, such as the protein or peptide, linked to the hydrogel polymer via the acrylic linker includes an enzyme digestion site such that the biomolecule, once inside the body of a subject, can be fully or partially removed from the hydrogel by enzymes naturally presented in the subject. In some embodiments, the enzyme digestion site is a peptide sequence cleavable by one or more proteases. Non-limiting examples of peptide sequence cleavable proteases are listed below:

| Peptide Sequences | Cleavable by |
|---|---|
| GPQGIWGQ (SEQ ID NO: 22) | Collagenases |
| PQGLA (SEQ ID NO: 23) | Matrix metallopeptidase |
| | 13 (MMP-13) |
| ELAPLRAP (SEQ ID NO: 24) | Plasmin |
| FPLRMRDW (SEQ ID NO: 25) | |
| KHGKV (SEQ ID NO: 26) | |
| EGTKKGHK (SEQ ID NO: 27) | |
| KKGHKLHL (SEQ ID NO: 28) | |
| HPVGLLARNRV | |
| (SEQ ID NO: 29) | |
| PVGLIG | Matrix metallopeptidase 2 |
| (SEQ ID NO: 30) | (MMP-2) |
| | Matrix metallopeptidase 9 |
| | (MMP-9) |
| CGGGPALLREGGGC | Matrix metallopeptidase 7 |
| (SEQ ID NO: 31) | (MMP-7) |
| CRDTEGEARGSVIDRC | Aggrecanase |
| (SEQ ID NO: 32) | |

In some embodiments, the protein or peptide comprises at least one cysteine residue. In some embodiments, the protein or peptide is attached to the acrylic linker via a thiol Michael addition reaction between a thiol group of the at least one cysteine residue and an alkenyl group in the acrylic linker.

In some embodiments, a concentration of the biomolecule in the hydrogel ranges from about 0.02 mM to about 20 mM, such as from about 0.05 mM to about 10 mM, from about 0.1 mM to about 7.5 mM, from about 0.2 mM to about 5 mM, from about 0.5 mM to about 4 mM, or from about 1 mM to about 3 mM. In some embodiments, the concentration of the biomolecule in the hydrogel is about 0.02 mM, about 0.05 mM, about 0.1 mM, about 0.2 mM, about 0.3 mM, about 0.5 mM, about 0.75 mM, about 1 mM, about 1.5 mM, about 2 mM, about 3 mM, about 4 mM, about 5 mM, about 7.5 mM, about 10 mM, about 12.5 mM, about 15 mM, about 20 mM, or any ranges therebetween. Varying the concentrations of the biomolecule in the hydrogel affects various aspects of the cell in contact with the hydrogel, depending the types of the attached biomolecules, such as survival, proliferation, morphology, protein expression, differentiation, tissue formation, and the like. As described elsewhere herein, the mechanical properties (such as storage modulus, elastic modulus, or gelation time) of the hydrogel herein is not significantly altered by the inclusion of or concentration change to the biomolecule. This is desirable as the mechanical properties of the hydrogel was also found to affect, for example, the survival, proliferation, morphology, protein expression, differentiation, tissue formation of the cells in contact with the hydrogel.

As described elsewhere herein, the present study developed various method of modulating the mechanical properties of the hydrogel, such as by changing the amount of the hydrogel polymer, degrees of crosslinking, ratios of the crosslinkers, and the like. Since the mechanical properties of the hydrogel affect, for example, the survival, proliferation, morphology, protein expression, differentiation, tissue formation of the cells in contact with the hydrogel, the ability to fine-tune various parameters of the mechanical properties are highly desirable.

In some embodiments, a storage modulus of the hydrogel ranges from about 500 Pa to about 25,000 Pa, such as about 1,000 Pa to about 20,000 Pa, from about 1,500 Pa to about 16,000 Pa, from about 1,500 Pa to about 4,000 Pa, from about 4,000 Pa to about 10,000 Pa, or from about 10,000 Pa to about 16,000 Pa. In some embodiments, the storage modulus of the hydrogel is about 500 Pa, 1,000 Pa, about 1,500 Pa, about 2,000 Pa, about 2,500 Pa, about 3,000 Pa, about 4,000 Pa, about 5,000 Pa, about 6,000 Pa, about 7,000 Pa, about 8,000 Pa, about 9,000 Pa, about 10,000 Pa, about 11,000 Pa, about 12,000 Pa, about 13,000 Pa, about 14,000 Pa, about 15,000 Pa, about 16,000 Pa, about 18,000 Pa, about 20,000 Pa, about 22,500 Pa, about 25,000 Pa or any ranges therebetween. In some embodiments, the storage modulus of the hydrogel described in this paragraph is the storage modulus at 37° C. In some embodiments, the storage modulus of the hydrogel described in this paragraph is the storage modulus at room temperature, such as 20° C. or 25° C.

In some embodiments, an elastic modulus of the hydrogel ranges from about 1 kPa to about 75 kPa, such as from about 1.5 kPa to about 60 kPa, from about 2 kPa to about 50 kPa, from about 2 kPa to about 15 kPa, from about 15 kPa to about 35 kPa, or from about 35 kPa to about 50 kPa. In some embodiments, the elastic modulus is about 1 kPa, about 1.5 kPa, 2 kPa, about 5 kPa, about 10 kPa, about 15 kPa, about 20 kPa, about 25 kPa, about 30 kPa, about 35 kPa, about 40 kPa, about 45 kPa, about 50 kPa, about 60 kPa, about 75 kPa, or any ranges therebetween. In some embodiments, the elastic modulus of the hydrogel described in this paragraph is the elastic modulus at 37° C. In some embodiments, the elastic modulus of the hydrogel described in this paragraph is the elastic modulus at room temperature, such as 20° C. or 25° C.

In some embodiments, a storage modulus of the hydrogel is higher than a loss modulus of the hydrogel at 37° C. In some embodiments, the storage modulus of the hydrogel is higher than a loss modulus of the hydrogel at room temperature, such as 20° C. or 25° C.

In some embodiments, the hydrogel further includes a cell. In some embodiments, the cell is a stem cell. In some embodiments, the cell is a stem cell that is able to differentiate and form a tissue. In some embodiments, the cell is a mesenchymal stem cell. In some embodiments, the cell is a mammalian cell. In some embodiments, the cell is a human cell.

### Hyaluronic Acid-Based Hydrogel

In some aspects, the present invention is directed to a hyaluronic acid (HA)-based hydrogel.

In some embodiments, the HA-based hydrogel includes a first HA polymer comprising a first crosslinker. In some embodiments, the HA-based hydrogel includes a second HA polymer comprising a second crosslinker. In some embodiments, the HA-based hydrogel includes a biomolecule attached to the first HA polymer or the second HA polymer through an acrylic linker attached to a hydroxyl group in the first HA polymer or the second HA polymer. In some embodiments, the HA-based hydrogel includes water.

In some embodiments, except for the first HA polymer and the second HA polymer, the HA-based hydrogel is the same as or similar to the hydrogels described elsewhere herein, such as in the “Hydrogel” section.

In some embodiments, the first HA polymer or the second polymer is replaced with gelatin.

### Method of Preparing Hydrogel

In some aspects, the instant specification is directed to a method of preparing a hyaluronic acid (HA)-based hydrogel.

In some embodiments, the method includes attaching the first crosslinker to the first HA polymer. In some embodiments, the method includes attaching the second crosslinker to the second HA polymer. In some embodiments, the method includes attaching the acrylic linker to the hydroxyl group of the first HA polymer or the second HA polymer. In some embodiments, the method includes attaching the biomolecule to the acrylic linker. In some embodiments, the method includes mixing the first HA polymer attached with the first crosslinker, the second HA polymer attached with the second crosslinker and water. In some embodiments, the method includes crosslinking the first crosslinker and the second crosslinker.

In some embodiments, the HA-based hydrogel is the same as or similar to the HA-based hydrogels described elsewhere herein, such as in the “Hyaluronic Acid-Based Hydrogel” section.

### Method of Promoting Tissue Regeneration

In some aspects, the present invention is directed to a method of promoting regeneration of a tissue in a subject in need thereof.

In some embodiments, the method includes: placing a hydrogel or a HA-based hydrogel at a site in need of regeneration in the subject.

In some embodiments, the hydrogel is the same as or similar to those described elsewhere herein, such as in the “Hydrogel” section.

In some embodiments, the HA-based hydrogel is the same as or similar to those described elsewhere herein, such as in the “Hyaluronic Acid-Based Hydrogel” section.

In some embodiments, the hydrogel or the HA-based hydrogel is formed at the site in need of regeneration.

In some embodiments, the hydrogel or the HA-based hydrogel is formed at the site in need of regeneration by injecting a liquid mixture for forming the hydrogel or the HA-based hydrogel into the site.

In some embodiments, the mixture undergoes crosslinking and gelates (gels) to form the hydrogel or the HA-based hydrogel spontaneously at the site.

In some embodiments, the tissue in need of regeneration is a bone tissue, a cartilage tissue, or combinations thereof.

In some embodiments, the biomolecule attached to the hydrogel polymer, the first HA polymer or the second HA polymer is bone morphogenetic protein 2 (BMP-2), or a functional mimicking peptide of BMP-2.

In some embodiments, the functional mimicking peptide of BMP-2 comprises at least one of the following amino acid sequences:

(SEQ ID NO: 8)
GCGGGDWIVAG,
(SEQ ID NO: 9)
NSVNSKIPKACCVPTELSAI,
or
(SEQ ID NO: 10)
KIPKASSVPTELSAISTLYL.

In some embodiments, the hydrogel or the HA-based hydrogel is placed in a femur of the subject.

In some embodiments, the subject is suffering from osteoporosis.

In some embodiments, the subject is a mammal, such as a human.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1-1

Hyaluronic acid (HA) hydrogels are biocompatible and feature diverse physical and biochemical properties. Although photopolymerization reactions are commonly used to form HA hydrogels, these strategies form radicals that produce cytotoxic byproducts. While Diels-Alder reactions between norbornene (Nor) and tetrazine (Tet) can form hydrogels in the absence of light, self-forming Diels-Alder hydrogels are limited by a narrow range in mechanics and poor control over biochemical modifications. Here, HA macromers are modified with Nor (HANor) or Tet (HATet) which upon mixing click into covalently crosslinked HA Nor-Tet hydrogels. By varying total HA concentration and macromer ratios, hydrogels with broad elastic moduli (5 to 30 kPa) were obtained, and peptide functionalization (0 to 2 mM) has no effect on mechanics or gelation time. Human mesenchymal stem cells (MSCs) on 2D RGD-functionalized HA Nor-Tet hydrogels exhibit changes in spreading and cellular mechanosensing in a stiffness-dependent manner. MSCs injected in 3D bioactive HA Nor-Tet hydrogel solutions are also significantly more viable than MSCs injected in the absence of hydrogel, demonstrating the use of HA Nor-Tet hydrogels as carriers of viable cells and peptides. Biochemical and mechanical properties of self-forming HA Nor-Tet hydrogels can be independently tuned, and these hydrogels support 2D and 3D cell culture.

### Example 1-2

Hydrogels are three-dimensional and highly hydrated crosslinked polymer networks that are used in various biomedical applications including tissue engineering, drug delivery, and regenerative medicine. To synthesize hydrogels, free-radical photopolymerization reactions using visible or ultraviolet light are commonly used due to fast gelation under physiological conditions. Depending on the moieties present, hydrogels can be formed via chain-growth or step-growth photopolymerization. Free-radical chain-growth photopolymerization of acrylated macromers form hydrogels with polydisperse kinetic chains, resulting in local differences in crosslink density, which introduces heterogeneity that could unpredictably influence cell-hydrogel interactions of encapsulated cells. In contrast, free-radical step-growth photopolymerization between molecules containing thiol and vinyl(ene) groups result in one-to-one click reactions that form hydrogels with a homogenous network structure. Despite their broad use, free-radical photopolymerization reactions require light curing systems that may be inaccessible to some research groups, and have additional drawbacks. In the presence of oxygen, radicals created during photopolymerization form reactive oxygen species (ROS) as a byproduct that could damage the DNA of encapsulated cells. The wavelength and intensity of light used for photopolymerization reactions must also be accounted for, since ultraviolet light can increase oxidative stress and lead to unwanted changes in cellular phenotype.

Alternatives to hydrogel photopolymerization rely on other catalysts including pH, electrostatic interactions, and temperature. For example, Michael-addition reactions between thiols and acrylates form hydrogels with tunable mechanical properties, and gelation rates can be decreased with increasing pH. While Michael-addition reactions are amenable to 3D cell culture, viability of encapsulated cells is typically compromised due to the need for high pH buffers, limiting their use to either acellular or 2D cell culture. Alginate is an anionic biopolymer that forms ionically crosslinked hydrogels when mixed with divalent cations (e.g., Ca²⁻ or Zn²⁻). Although ionically crosslinked hydrogels are biocompatible, hydrogel networks held by electrostatic interactions have low mechanical properties and are unstable due to the diffusion of divalent cations over time. Thermoresponsive hydrogels transition from liquid to hydrogel above a lower critical solution temperature (LCST) or below an upper critical solution temperature (UCST). Thermosensitive poly(N-vinylcaprolactam) (PNVCL) hydrogels are liquid at room temperature and gel at a physiologic LCST. Chondrocytes and mesenchymal stem cells encapsulated in these hydrogels exhibit high viability and cartilage extracellular matrix formation in vitro and in vivo. Gelatin is a thermoresponsive polymer that has a concentration-dependent UCST of ˜25 to 30° C., which is not suitable for in vivo applications but could be leveraged for additive manufacturing processes. Although not many synthetic polymers with physiologic UCSTs exist, poly(N-acryloyl glycinamide) (PNAGA) hydrogels gel when cooled to 37° C. as a potential injectable drug-releasing material.

While catalyst-based hydrogel polymerization reactions can create hydrogels with myriad properties, these hydrogel techniques rely on an external input (e.g., light, pH, cations, temperature), which limits their use to conditions where a catalyst is present. Diels-Alder reactions are highly specific cycloadditions between dienes and dienophiles that do not require light or other stimuli in aqueous environments. For example, hyaluronic acid (HA) macromers modified with furan (diene) were reacted with di-maleimide (dienophile) poly(ethylene glycol) (PEG) crosslinkers to create self-forming hydrogels. Since furan-maleimide hydrogels take several hours to form at a neutral pH, these hydrogels are typically synthesized in acidic conditions not suitable for 3D cell culture. By adding an electron-donating methyl group to furan dienes, methylfuran-dialdehyde hydrogels can be synthesized in under 15 minutes at a physiologic pH. However, a major limitation of Diels-Alder hydrogels formed between furans and maleimides is that these reactions are reversible and prone to hydrolysis, resulting in uncontrollable changes to hydrogel properties including swelling, stiffness, and porosity. By using electron-rich cyclic fulvenes in lieu of furan dienes, Madl & Heilshorn formed more stable fulvene-maleimide hydrogels that support 3D cell culture.

Although these Diels-Alder reactions can create biocompatible, self-forming hydrogels, hydrogel mechanics are low (storage modulus, G′<1,000 Pa) and gelation times are high (upwards of 3 hours). Diels-Alder reactions between tetrazine (Tet, diene) and norbornene (Nor, dienophile) are irreversible and offer an alternative chemistry for creating self-forming hydrogels with tunable properties. Hydrogels that use this chemistry were first reported by Alge et. al, by reacting 4-arm PEG-Tet with di-Nor peptides. By including Nor-modified RGD, biocompatible self-forming hydrogels with adhesive domains were created, with storage moduli ranging from 225 to 2,345 Pa by varying PEG-Tet concentration. In another study, by modifying alginate biopolymers with Nor or Tet miotics (5% Nor and Tet substitution), hydrogels with increased mechanical properties (elastic modulus ˜15 kPa for 4% w/v hydrogels) were formed. Although these hydrogels are structurally stable and support 3D cell culture, hydrogel polymerization is slow (>1 hour), which limits their clinical use and could cause an inhomogeneous cell distribution during gelation. By using gelatin macromers with higher Nor (GelNor) and Tet (GelTet) substitution (˜20%), Koshy et al. synthesized rapid self-forming hydrogels that gel as little as ˜5 minutes with a G′ of ˜4,000 Pa.

It was hypothesized that by increasing the degree of substitution of Nor and Tet moieties, self-forming hydrogels with fast gelation times and superior mechanics can be achieved. It was also hypothesized that HA pre-coupled with peptides can be used to incorporate bioactive motifs to HA Nor-Tet hydrogels without impacting mechanical properties or gelation rates. In this study, carboxyl groups of HA macromers were modified with Nor (HANor, 50% substitution) or Tet (HATet, 40% substitution) to create self-forming HA Nor-Tet hydrogels. Mechanical properties and gelation kinetics were controlled by tuning the total macromer concentration and the stoichiometric ratio between HANor and HATet macromers. By modifying HANor hydroxyl groups with methacrylates (HANorMe), thiol-containing peptides were pre-coupled to methacrylates in HANorMe macromers and used to form peptide-functionalized HA Nor-Tet hydrogels. Using this platform, the present study investigated cellular mechanosensing of mesenchymal stem cells (MSCs) on 2D RGD-functionalized HA Nor-Tet hydrogels and the protective effects of HA Nor-Tet hydrogel solutions on MSCs injected from needles of clinically relevant dimensions.

### Example 1-3: Macromer Synthesis and HA Nor-Tet Hydrogel Polymerization

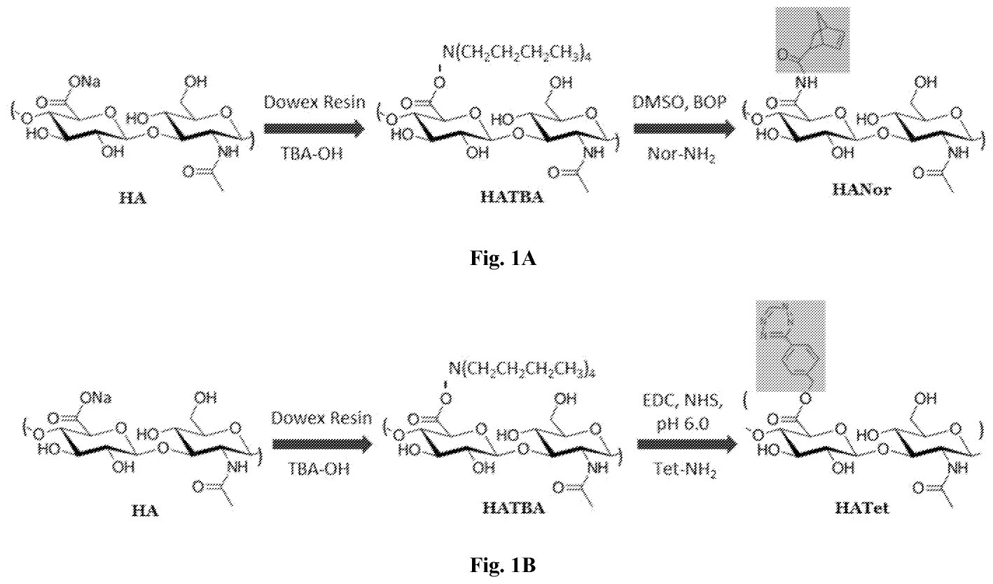

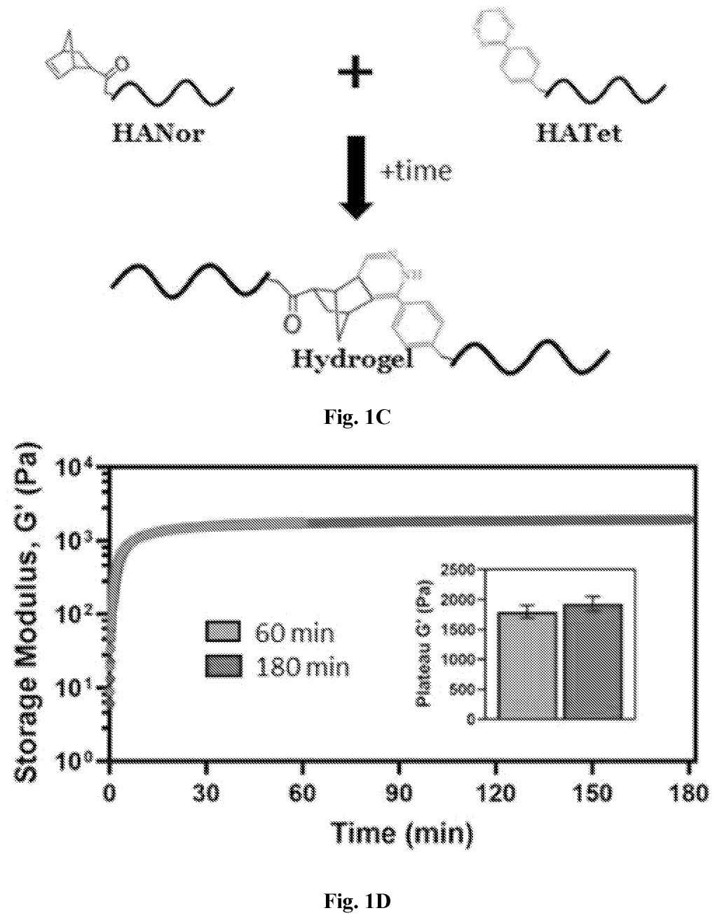

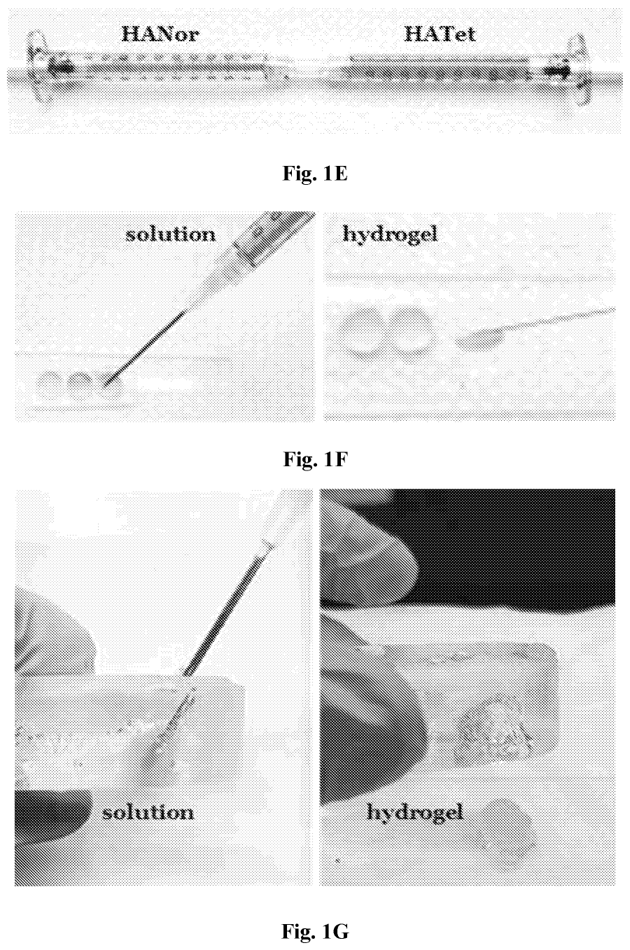

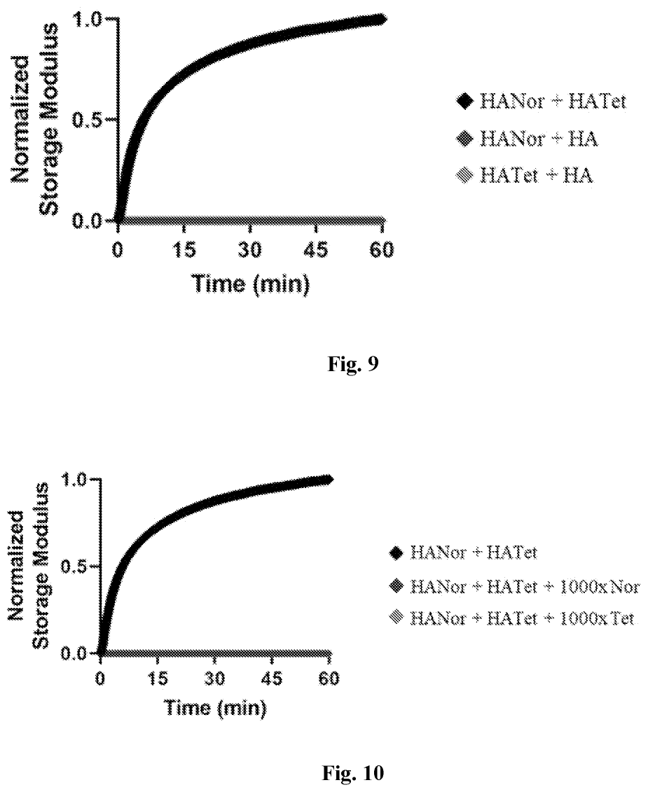

HANor macromers were synthesized by converting sodium hyaluronate (HA) to its tetrabutylammonium salt (HATBA), followed by an anhydrous benzotriazole-1-yl-oxy-tris (dimethylamino)-phosphonium hexafluorophosphate (BOP) reaction in dimethyl sulfoxide (DMSO) between carboxyl groups in HATBA and amines in norbornene amine (Nor-NH2) (FIG. 1A). (Gramlich et al., Biomaterials 2013, 34, 9803). HATet macromers were synthesized by reacting carboxyl residues in HATBA with amines in tetrazine amine (Tet-NH₂) in the presence of 1-(3-dimethylaminopropyl)-3-ethylcarbodiimide (EDC) and N-hydroxysuccinimide (NHS) (FIG. 1B). (Desai et al., Biomaterials 2015, 50, 30 and Koshy et al., Adv. Healthc. Mater. 2016, 5, 541) Upon mixing, HANor and HATet macromers self-form into stable HA Nor-Tet hydrogels (FIG. 1C). At 37° C., the storage modulus (G′) of 2% w/v hydrogels after 60 (1,800±100 Pa) and 180 (1,930±120 Pa) mins is not statistically significant (FIG. 1D), demonstrating that HA Nor-Tet hydrogels self-form in under an hour. To evaluate the specificity of Diels-Alder reactions between HANor and HATet, two tests were performed. HANor or HATet were first mixed with unmodified HA, resulting in no hydrogel formation (FIG. 9). HANor and HATet were also mixed in the presence of either Nor-NH₂ or Tet-NH₂ molecules in excess (1,000×). Here, the unbound Nor and Tet molecules bind to their respective Diels-Alder partners, preventing hydrogels from forming (FIG. 10). This demonstrates that the Diels-Alder reactions between Nor and Tet are highly specific and necessary for HA Nor-Tet hydrogels to self-form.

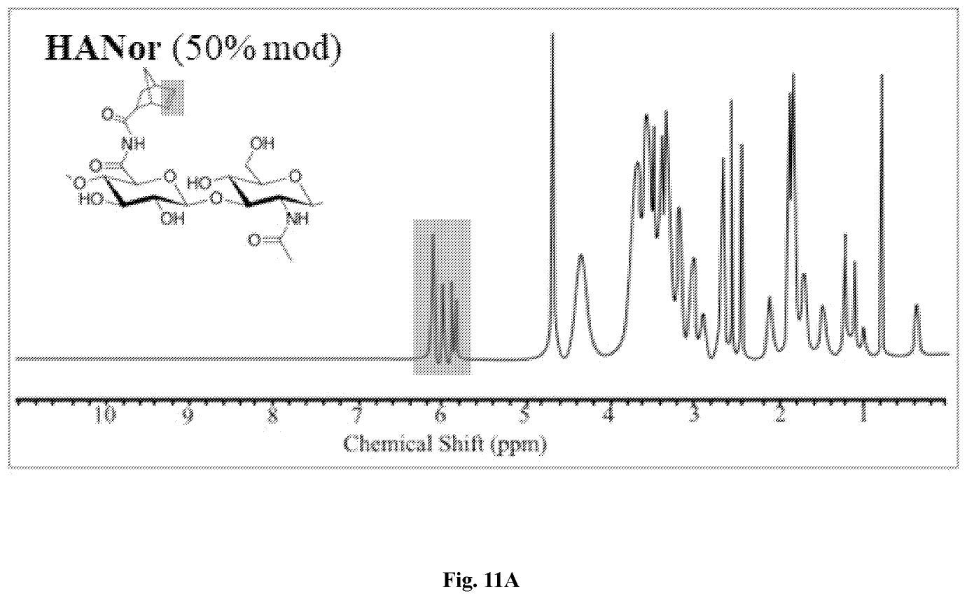

The present study next sought to determine how the inclusion of Nor and Tet moieties could impact fluid viscosity of HANor and HATet macromers in solution since this is an important parameter for developing injectable materials and bioinks for 3D printing. Alge et al. showed that solutions containing gelatin functionalized with 20% Nor and Tet pendant groups were less viscous than solutions of unmodified gelatin, and this may be due to the presence of Nor and Tet interrupting with physical interactions required for the thermogelation of gelatin. HANor and HATet macromers used in this study have approximately 50% and 40% of their repeat units functionalized with Nor and Tet, respectively, as confirmed by 1H NMR (400 MHZ, D₂O) (FIGS. 11A-11B), which is significantly higher than Nor and Tet substitutions used by other Diels-Alder Nor-Tet hydrogels. At 37° C., the viscosity of unmodified HA ranges from 3.3 to 76.2 mPa s for 2% to 6% w/v solutions (Table 1-1). Nor and Tet substitutions result in a modest decrease in viscosity for 2% and 4% w/v HANor and HATet solutions, and a much larger decrease in viscosity for 6% w/v HANor (37.8 mPa s) and HATet (36.3 mPa s) solutions (Table 1-1). The presence of Nor and Tet results in a decrease in viscosity, and the viscosities for 2%, 4%, and 6% w/v HANor and HATet solutions are well below the upper limit of viscous bioinks and fluids (>300 mPa s) that can result in needle clogging and heterogeneous mixing post-hydrogel extrusion.

TABLE 1-1
Viscosity of polymers used in hydrogel formation at 37°
C. Viscosities of HA, HANor, and HATet at 2, 4, and 6% w/v

| Polymer | 2% w/v [mPa s] | 4% w/v [mPa s] | 6% w/v [mPa s] |
|---|---|---|---|
| HA | 3.3 ± 0.7 | 17.3 ± 2.7 | 76.2 ± 10.2 |
| HANor | 3.1 ± 0.5 | 16.4 ± 2.4 | 37.8 ± 5.6 |
| HATet | 2.4 ± 0.4 | 15.1 ± 1.7 | 36.3 ± 4.7 |

HANor and HATet macromers can be dissolved at room temperature in aqueous media, loaded into a syringe, and mixed with a syringe coupler (FIG. 1E). Upon mixing, the hydrogel solution will begin to polymerize but allows for sufficient time to extrude through a needle that will self-form into a hydrogel that conforms into any shape ranging from cylindrical molds (FIG. 1F) to amorphous cavities (FIG. 1G). Gelation time is an important parameter of self-forming hydrogels and ideally, injectable hydrogels should polymerize within a few minutes post-injection under physiological conditions. For in vivo applications, hydrogels that polymerize too slowly could diffuse into surrounding tissues, leading to the unwanted presence of hydrogel material away from the target site, whereas hydrogels that form too quickly can polymerize prematurely in the needle extruder prior to reaching the targeted site. HA Nor-Tet hydrogels with 2% to 6% w/v macromer concentration can be readily dissolved and mixed with sufficient time to extrude to a region of interest (e.g., molds, cavities).

### Example 1-4: Physical Properties of Self-Forming HA Nor-Tet Hydrogels are Highly Tunable

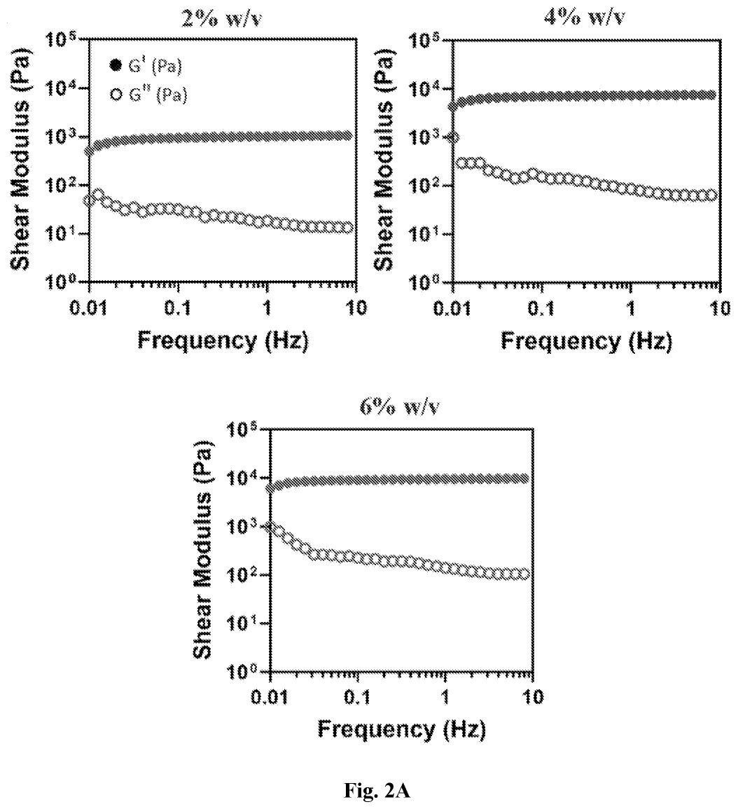

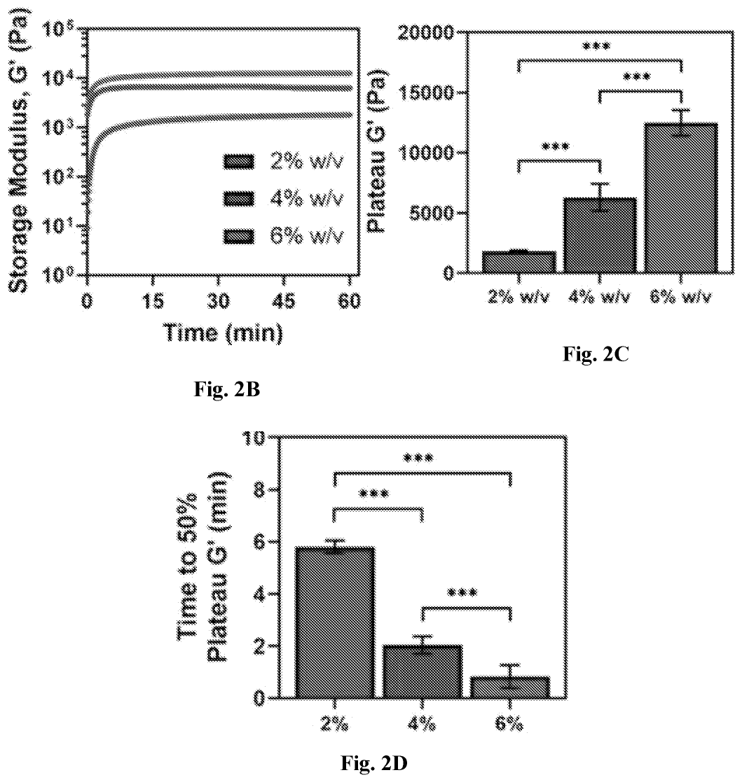

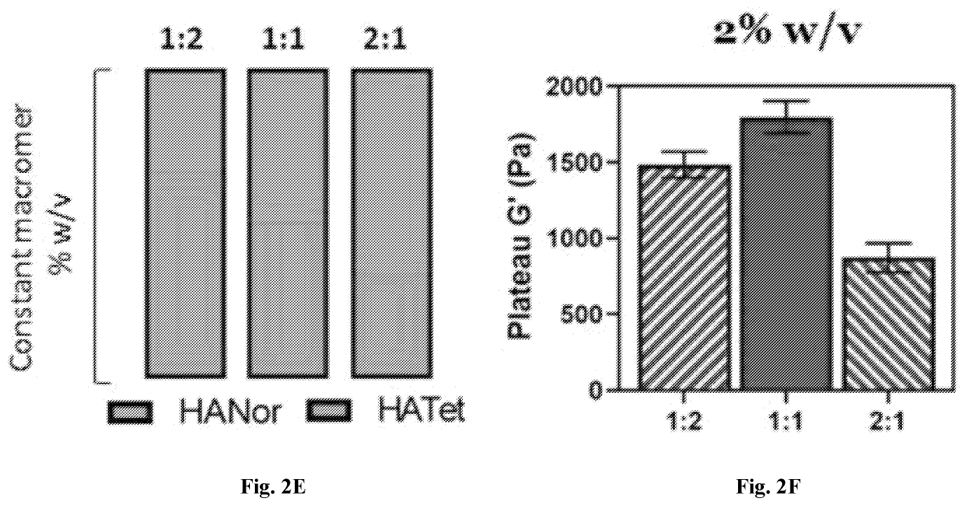

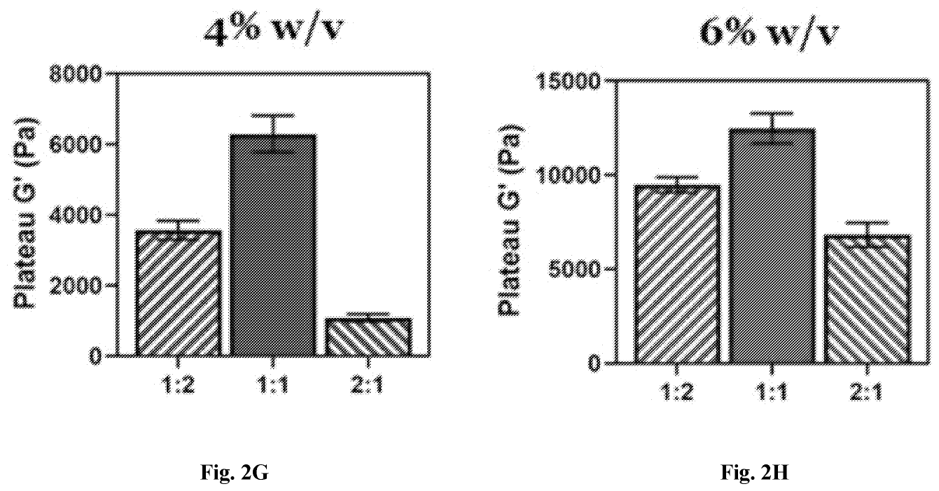

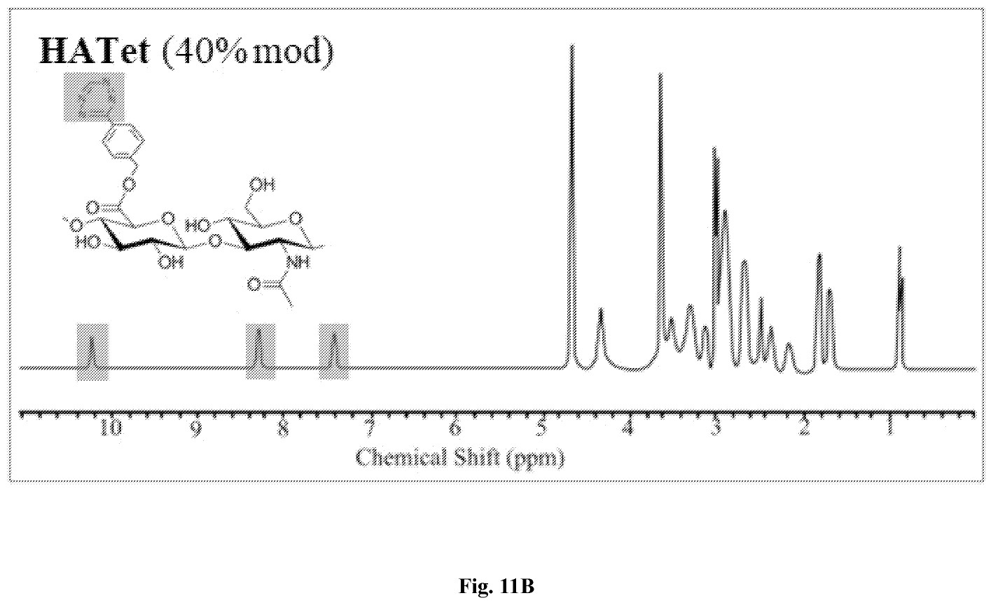

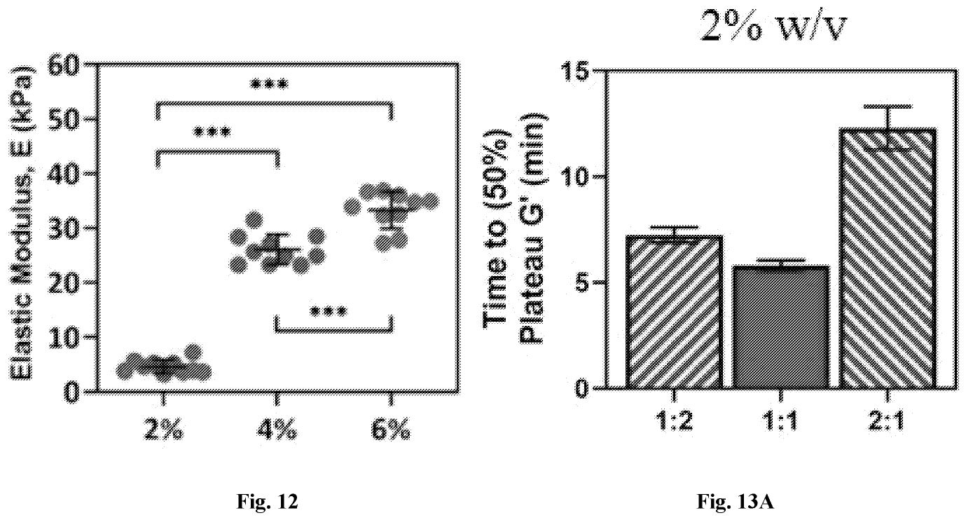

To evaluate mechanics and gelation properties of self-forming hydrogels, HANor and HATet macromers were mixed at 2%, 4%, or 6% total w/v macromer concentration. Frequency sweep rheology (0.1 to 10 Hz) at 37° C. shows that the storage modulus (G′) is above the loss modulus (G″) and that G′ is constant across a range of oscillatory frequencies (FIG. 2A), demonstrating the formation of stable hydrogels post-mixing. HA Nor-Tet hydrogel mechanics also increase with increasing macromer concentration (FIG. 2B). The range in hydrogel mechanics achieved is large, with G′ values of 1,800±100 Pa (2% w/v), 6,300±1,000 Pa (4% w/v), and 12,500±1,100 Pa (6% w/v) (FIG. 2C). To determine the elastic modulus (E), cylindrical HA Nor-Tet hydrogels (8 mm diameter, 2 mm height) were subject to compression testing, with E values ranging from ˜5 kPa (2% w/v) to ˜30 kPa (6% w/v) (FIG. 12). Gelation rates also decreased with increasing mechanics. For 2% w/v HA Nor-Tet hydrogels, it took 5.80±0.24 min to reach 50% of their final G′, and gelation time decreased for 4% w/v (2.04±0.34 min) and 6% w/v (0.83±0.44 min) hydrogels (FIG. 2D).

The range in HA Nor-Tet hydrogel mechanics achieved by simply increasing the total macromer concentration is significantly larger than previous systems that use a Diels-Alder chemistry (Table 1-2). This could be due to irreversible bonds formed between Nor and Tet and the high degree of Nor and Tet substitution in the HANor and HATet macromers. Diels-Alder reactions between furan or methylfuran and maleimides are reversible, resulting in hydrogels that are unstable over time with low mechanical properties. While replacing furans with fulvenes increases the stability of self-forming hydrogels, the mechanical properties are still low (<1,000 Pa), possibly due to the small number of reactive groups per component. Additionally, the reaction rates between furans or methylfurans and maleimides are inherently slow. By using tetrazine as the diene with norbornene as the dienophile, irreversible bonds form during gelation, resulting in mechanically stable, biocompatible self-forming hydrogels. It was hypothesized that increasing the interactions between Nor and Tet moieties will result in hydrogels with tunable mechanics that polymerize in a short amount of time. By using HANor and HATet macromers with a high degree of Nor (50%) and Tet (40%) substitution, rapidly forming hydrogels with a large range of mechanics were formed, and it is expected that an even broader range of mechanical properties is possible by increasing the number of Nor and Tet moieties, either by further increasing the degree of substitution, or by using higher molecular weight HA macromers.

TABLE 1-2
Diene and dienophiles used in Diels-Alder reactions with
corresponding storage moduli (G′) and gelation times.

| Diene | Dienophile | Storage modulus, G′ | | Gelation time | | Reference |
|---|---|---|---|---|---|---|
| Furan | Di-Maleimide | <1,000 | Pa | 120-180 | min | 26 |
| Methylfuran | Di-Maleimide | <1,500 | Pa | 15 | min | 27 |
| Furan | Maleimide | 5,000-35,000 | Pa | 15-170 | min | 28 |
| Fulvene | Maleimide | <1,000 | Pa | 6-35 | min | 29 |
| Tetrazine | Di-Norbornene | <2,500 | Pa | <10 | min | 30 |
| Tetrazine | Norbornene | <500 | Pa | 100 | min | 31 |
| Tetrazine | Norbornene | <4,500 | Pa | <15 | min | 32 |

To further explore the physical properties of self-forming HA Nor-Tet hydrogels, HANor and HATet were mixed at different stoichiometric ratios (HANor: HATet) at a constant total macromer concentration (FIG. 2E). For 2% w/v HA Nor-Tet hydrogels, the plateau G′ varied for different HANor: HATet ratios, with the highest mechanics observed at a 1:1 ratio (1,800±110 Pa), followed by 1:2 (1,500±90 Pa) and 2:1 (900±100 Pa) ratios (FIG. 2F). Similarly, for 4% and 6% w/v HA Nor-Tet hydrogels, highest mechanics were seen at a 1:1 ratio, with second and third highest G′ observed in 1:2 and 2:1 HANor: HATet ratios, respectively (FIGS. 2G and 2H). The range in mechanics by simply varying the HANor: HATet ratio was significant at a constant macromer concentration. In 4% w/t HA Nor-Tet hydrogels there was an over 5-fold change (1,100±110 to 6,300±500 Pa) and in 6% w/v HA Nor-Tet hydrogels an almost 2-fold change (6,800±700 to 12,500±800 Pa) in G′ between 2:1 and 1:1 HANor: HATet ratios. These findings demonstrate that mechanical properties can be independently controlled while maintaining biopolymer concentration constant. This is useful in tuning hydrogel mechanics without changing fluid properties (e.g., viscosity) that could impact extrusion parameters.

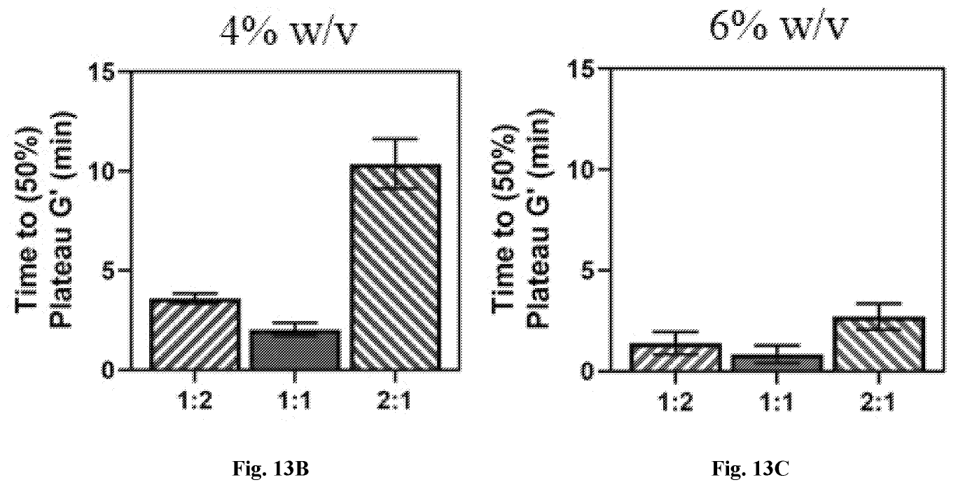

Interestingly, the rate of hydrogel formation also changed with varying HANor: HATet ratios at a constant biopolymer concentration. For 2% w/v HA Nor-Tet hydrogels, gelation time increased from 5.80 minutes at a 1:1 Nor: Tet ratio to 12.30 minutes for a 2:1 Nor: Tet ratio (FIG. 13A). The same trends were observed for 4% and 6% w/v HA Nor-Tet hydrogels, with the shortest gelation time seen at a 1:1 Nor: Tet ratio, with second and third longest gelation times at 1:2 and 2:1 Nor: Tet ratios, respectively (FIGS. 13B-13C). An increase in total macromer concentration corresponds to higher mechanics and a decrease in gelation kinetics, and this observation shows that changing the amount of Nor and Tet moieties at a fixed macromer concentration also regulates gelation time.

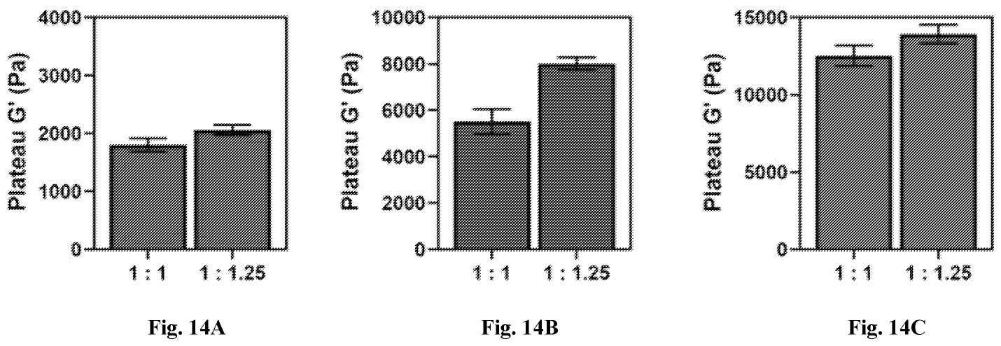

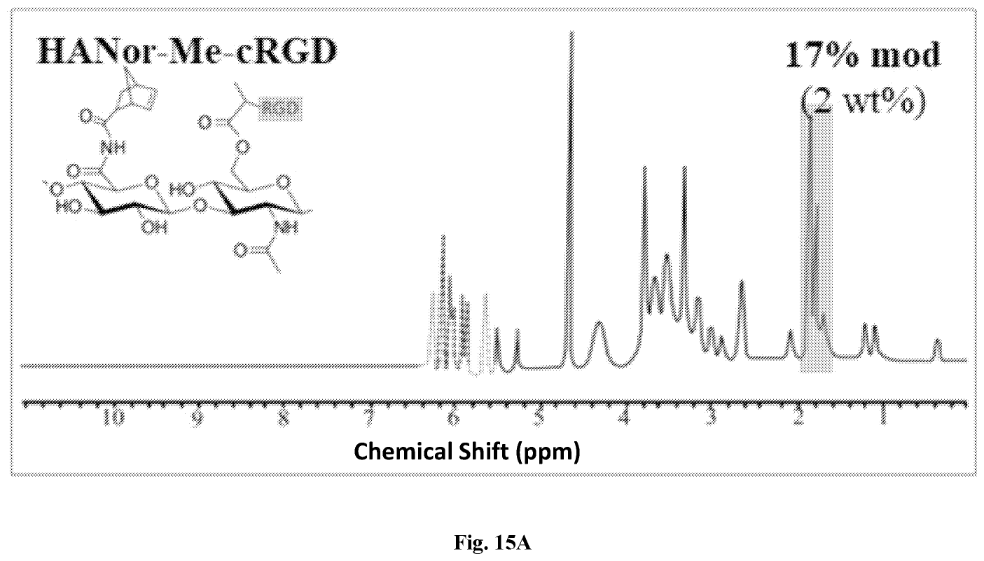

At a 1:1 Nor: Tet ratio, there is approximately 25% more Nor than Tet moieties, since the degree of substitution for the HANor and HATet macromers used is 50% and 40%, respectively. Thus, it is expected that the theoretical maximum stiffness (and minimum gelation time) would occur at a ratio where Nor and Tet moieties are equal. Based on 50% HANor and 40% HATet modifications, this corresponds to a 1:1.25 HANor: HATet ratio. At this optimal ratio, mechanics for 2%, 4%, and 6% w/v HA Nor-Tet hydrogels were higher than HA Nor-Tet hydrogels formed at a 1:1 HANor: HATet stoichiometric ratio (FIG. 14A-14C). The storage modulus G′ increased from 1,800 to 2,100 Pa for 2% w/v, 6,300 to 8,000 Pa for 4% v/w, and 12,500 to 14,000 Pa for 6% w/v HA Nor-Tet hydrogels. These findings show that Diels-Alder Nor-Tet reactions are highly specific, and that maximal mechanics are achieved when the amount of Nor and Tet moieties present is equal.

### Example 1-5: Self-Forming Hydrogels can be Peptide Functionalized without Changing Mechanics

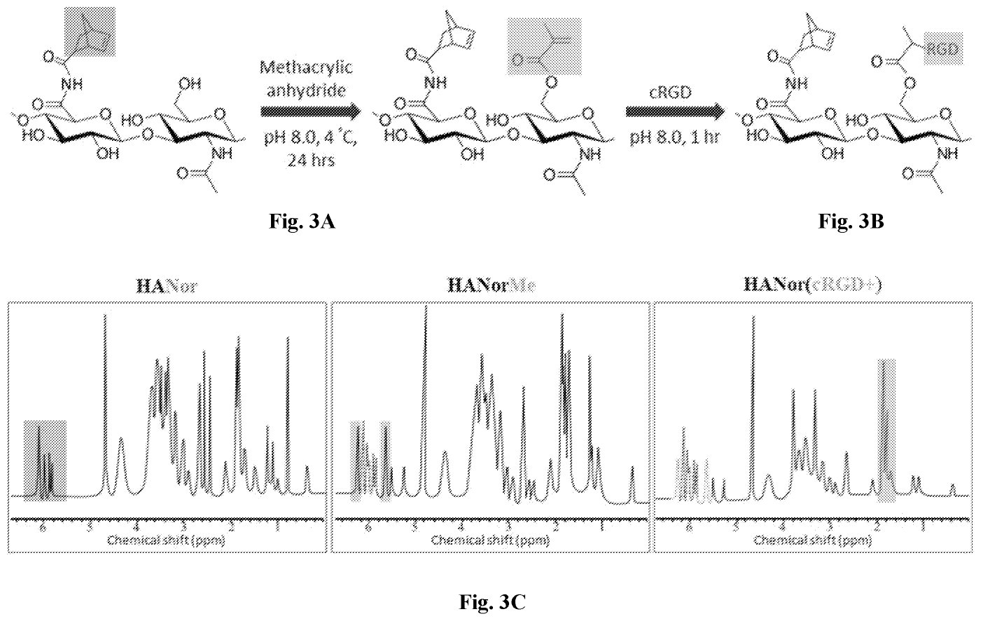

Conventional hydrogels formed with a Diels-Alder Nor-Tet chemistry cannot be peptide-functionalized without affecting crosslinking density or requiring a secondary photoreaction post-hydrogel polymerization. (Alge et al., Biomacromolecules 2013, 14, 949 and Desai et al., Biomaterials 2015, 50, 30). To functionalize Nor-Tet hydrogels with adhesive RGD peptides, Alge and coworkers bound Nor-RGD to multi-arm PEG-Tet, resulting in competition between Nor-RGD and di-Nor crosslinker peptides for available Tet sites. In another study, Desai et al. used a thiol-ene photopolymerization reaction to couple thiolated RGD peptides to pendant Nor moieties in pre-formed alginate Nor-Tet hydrogels. Here, HA Nor-Tet hydrogels with independent control over peptide functionalization were created by tethering thiolated RGD peptides to HANor via a two-step process (FIGS. 3A-3C).

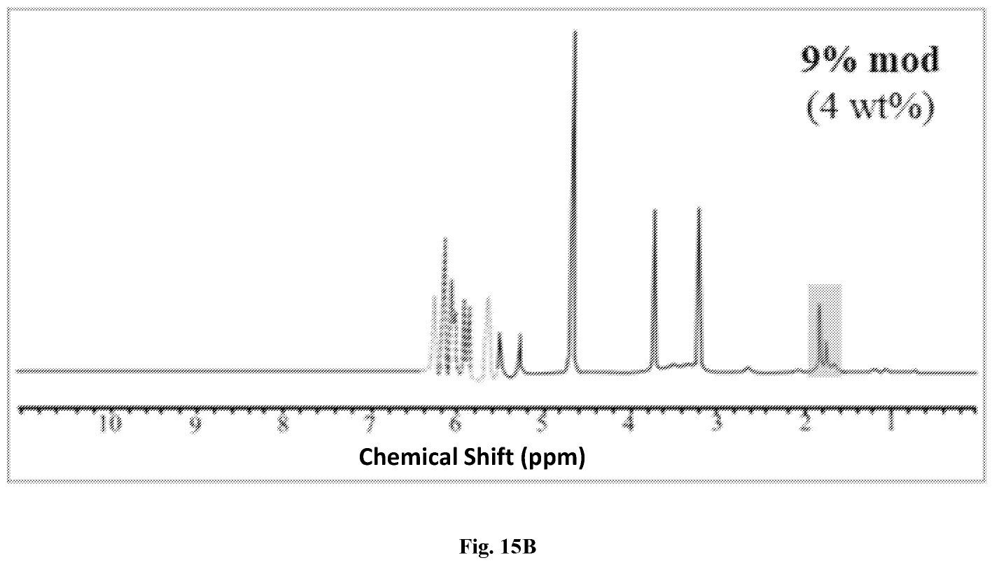

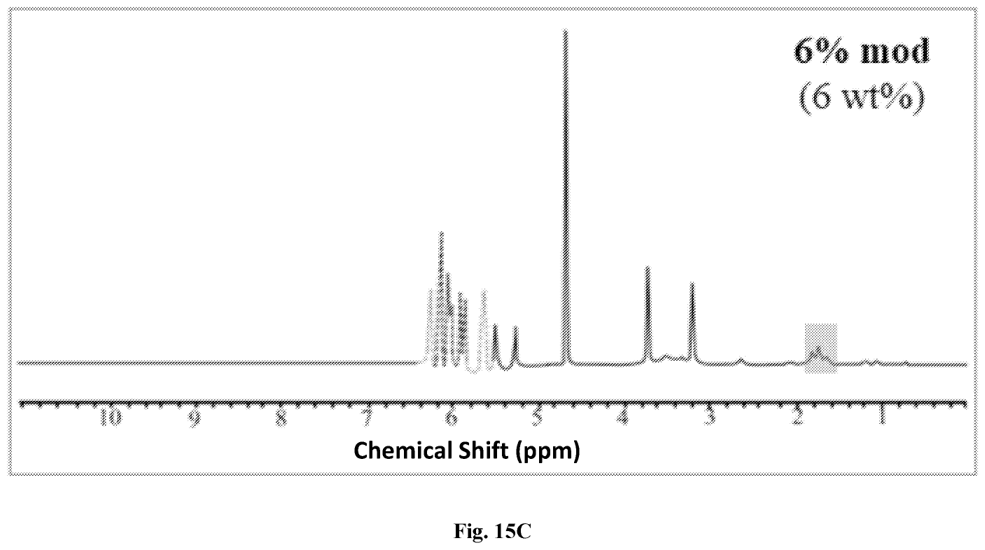

First, hydroxyl groups in HANor were modified with methacrylates (Me) via esterification with methacrylic anhydride (Smeds and Grinstaff, 2000, DOI 10.1002/1097-4636), resulting in an HANorMe macromer (FIG. 3A). Next, Me groups in HANorMe and thiols in thiolated RGD (cRGD) peptides were covalently bound via a Michael-addition reaction (Nair et al., Chem. Mater. 2013, DOI 10.1021/cm402180t), resulting in HANor (cRGD+) (FIG. 3B), an HANor macromer that is peptide-functionalized without altering Nor groups dedicated for HA Nor-Tet hydrogel formation. By varying the amount of cRGD reacted with a constant amount of HANor Me, the effective concentration of RGD was kept at 2 mM for 2%, 4%, and 6% w/v HA Nor-Tet hydrogels (Table 1-3). 1H NMR was used to confirm Nor (50%), Me (78%), and cRGD modification (FIG. 3C). To maintain a constant effective concentration, the amount of cRGD peptide added to HANorMe during synthesis was adjusted accordingly. Less peptide coupling to HANor Me should occur for increasing total macromer concentration, which was confirmed by 1H NMR to be 17%, 9%, and 6% cRGD modification for 2%, 4%, and 6% w/v HA Nor-Tet hydrogels, respectively (FIGS. 15A-15C). At least 60% of Me sites are available for coupling after functionalization with cRGD, and these unreacted Me moieties can be used to either increase the effective concentration of cRGD or modify biochemical properties by adding other thiolated peptides.

TABLE 1-3
Reaction parameters to create HANor(Pep+)
macromers with appropriate cRGD
(Sequence: GCGYGRGDSPG, SEQ ID NO: 33;
MW: 1,025 g mol⁻¹) concentrations to achieve
an effective concentration of 2 mM.

| Macromer w/v | HANorMe (mg) | cRGD (mg) |
|---|---|---|
| 2% | 100 | 20.5 |
| 4% | 100 | 10.3 |
| 6% | 100 | 6.9 |

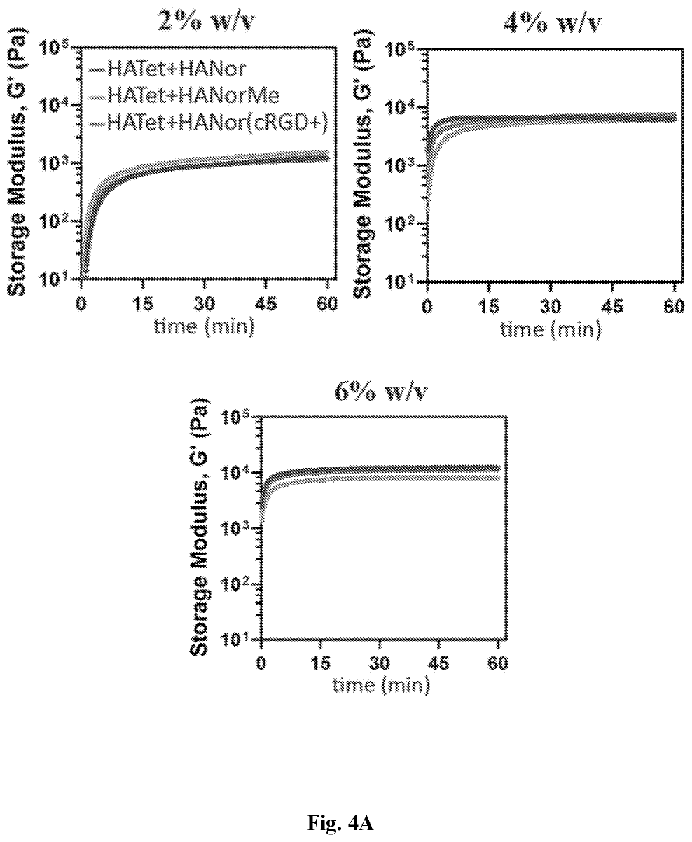

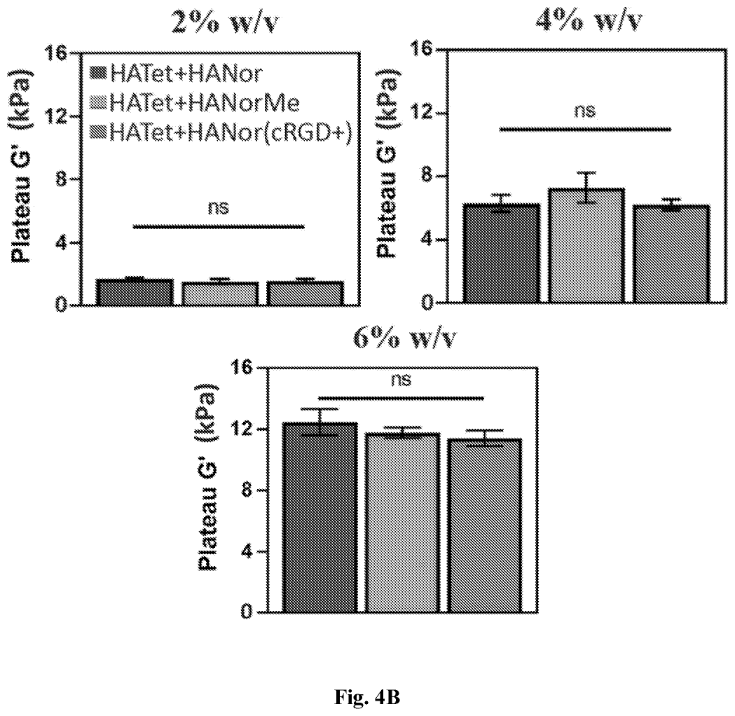

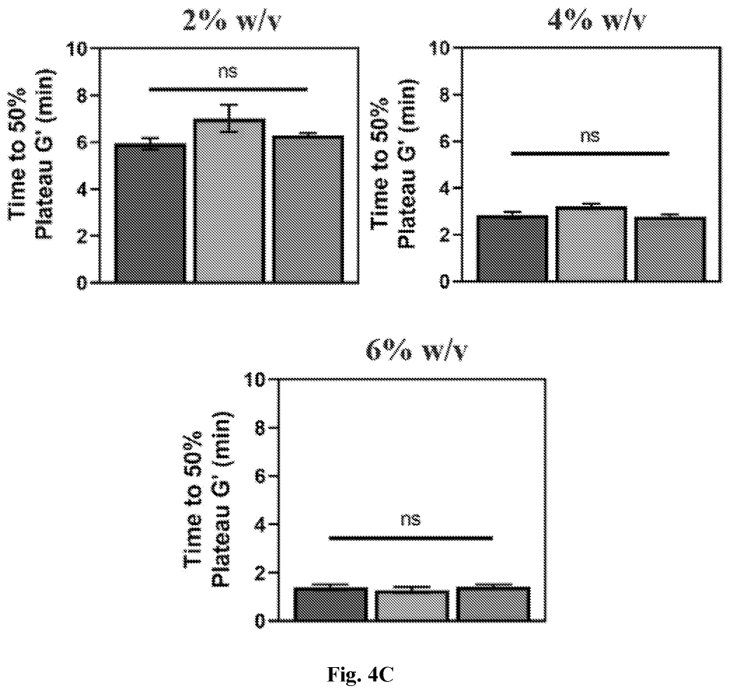

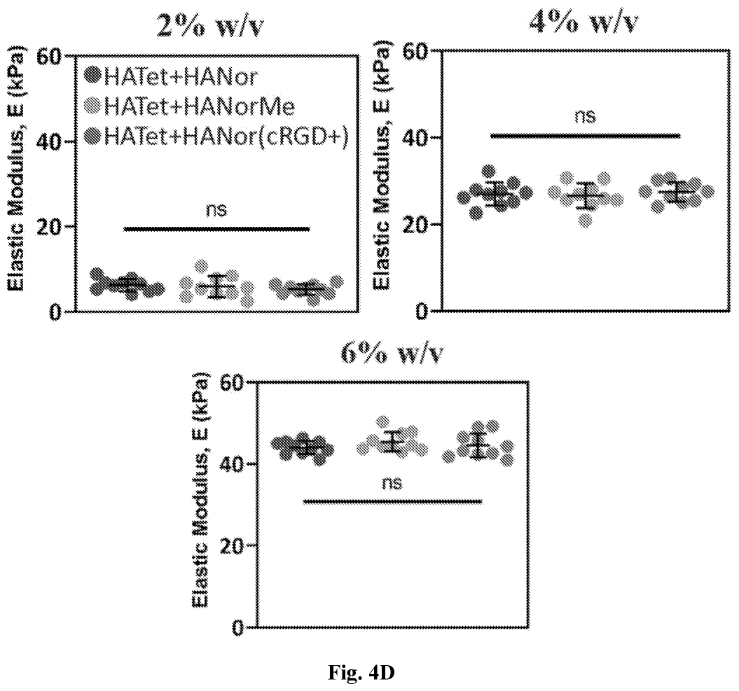

To confirm that peptide modifications to the hydroxyl group do not impact Nor moieties used for HA Nor-Tet binding, HATet was mixed with either HANor, HANorMe, or HANor (cRGD+) at 2%, 4%, or 6% total macromer w/v at a 1:1 macromer ratio. Time sweep rheology shows that G′ increases with increasing w/v, and that no discernable changes in gelation from Me substitution or RGD peptide functionalization are observed (FIG. 4A). Final mechanical properties (plateau G′) did not change across the macromer groups for 2% (1,800±110 Pa), 4% (6,300±1,000 Pa), and 6% (12,500±1,100 Pa) w/v HA Nor-Tet hydrogels (FIG. 4B). Additionally, peptide functionalization did not have a significant effect on gelation times, with times to 50% plateau G′ of 5.80±0.24, 2.04±0.34, and 0.83±0.44 minutes for 2%, 4%, and 6% w/v HA Nor-Tet hydrogels, respectively (FIG. 4C). Elastic moduli (E) of fully formed cylindrical hydrogels yielded consistent results across the different macromer groups for 2% (4.6±1.2 kPa), 4% (26.1±2.7 kPa), and 6% (33.3±3.4 kPa) w/v HA Nor-Tet hydrogels (FIG. 4D). These results show that HA Nor-Tet hydrogels can be biofunctionalized with thiolated peptides without affecting mechanical properties and gelation kinetics. Although cRGD was used, this procedure can be applied to any thiol-containing molecule. The effective peptide concentration can be easily tuned by varying the amount of peptide mixed with HANorMe during thiol-methacrylate peptide coupling.

### Example 1-6: Cells Adhere and Proliferate on RGD-Functionalized HA Nor-Tet Hydrogels

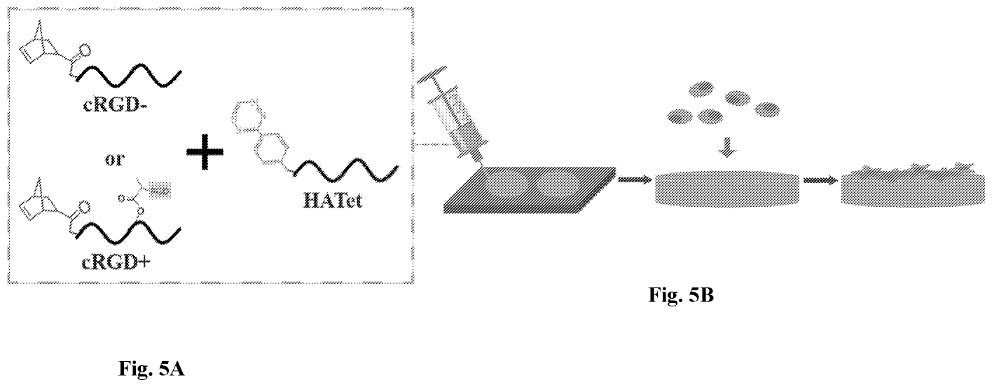

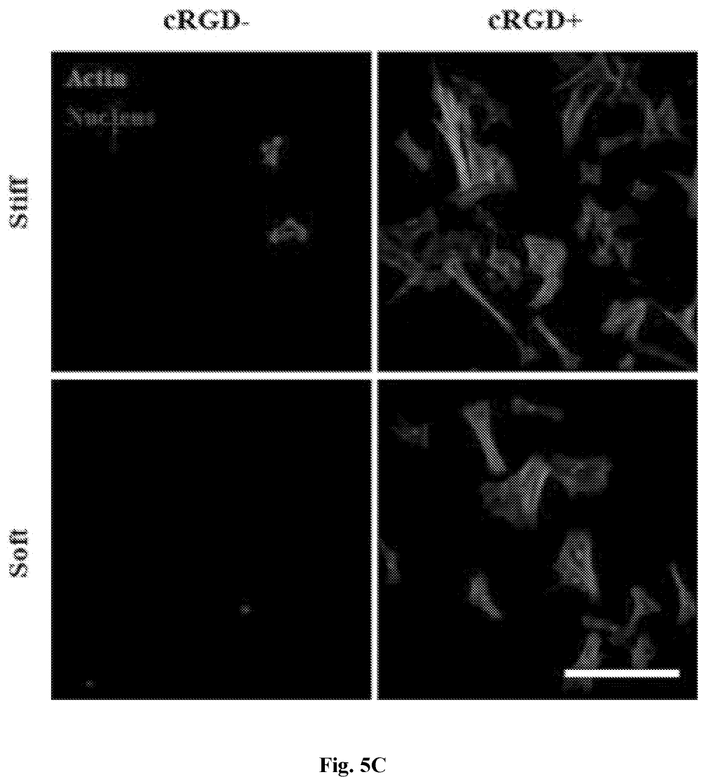

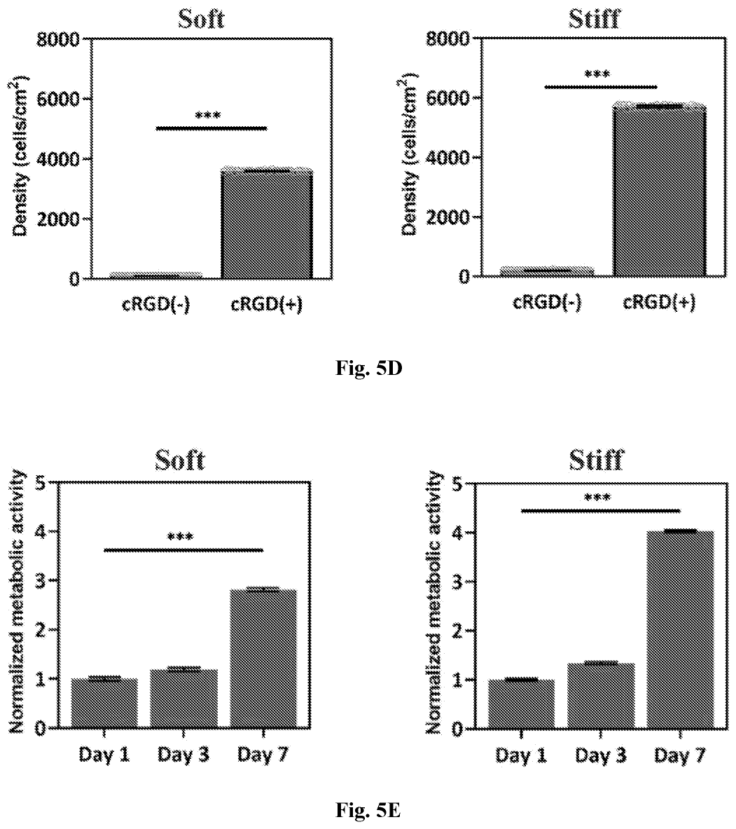

After demonstrating that HA Nor-Tet hydrogels can be independently functionalized with peptides without altering mechanics, their use as a biocompatible cell culture platform was investigated. Since cellular adhesion to unmodified HA hydrogels is poor, (Burdick et al., Adv. Mater. 2011, 23, 41 and Glass et al., Biomaterials 1996, 17, 1101) adhesive molecules need to either be passively adsorbed or chemically bound to HA hydrogels to support cell adhesion. To confirm the bioactivity of adhesive RGD peptides, HANor with or without coupled cRGD was mixed with HATet and extruded into silicone molds (8 mm diameter, 0.5 mm height), to form adhesive (cRGD)+) or non-adhesive (cRGD−) HA Nor-Tet hydrogels (FIG. 5A). Human mesenchymal stem cells (MSCs, 3,000 cells cm⁻²) were then cultured on top of Soft (2% w/v, 4.6 kPa E) or Stiff (6% w/t, 33.3 kPa E) self-forming HA Nor-Tet hydrogels (FIG. 5B) and cellular adhesion and proliferation was evaluated.

After three days in culture, representative fluorescent images of MSCs show minimal cell attachment on Soft and Stiff cRGD− hydrogels, while significant cell attachment is observed on cRGD+ Soft and Stiff HA Nor-Tet hydrogels (FIG. 5C). Quantification of cell density (number of cells cm⁻²) of MSCs on Soft HA Nor-Tet hydrogels confirms poor cell attachment on cRGD− hydrogels (100=5 cells cm⁻²), whereas significant attachment was observed on cRGD+ HA Nor-Tet hydrogels (3,600±20 cells cm⁻²) (FIG. 5D). Cell counts on Stiff HA Nor-Tet hydrogels is also higher for MSCs on cRGD+ (5,700±40 cells cm⁻²) hydrogels in comparison to cRGD−(201±11 cells cm⁻²) HA Nor-Tet hydrogels. The cell density on Soft and Stiff cRGD+ hydrogels was higher than the initial seeding density (3,000 cells cm⁻²), suggesting that MSCs adhere and proliferate on RGD-functionalized 2D HA Nor-Tet hydrogels. To evaluate cell proliferation over time, MSCs were cultured on Soft and Stiff cRGD+ HA Nor-Tet hydrogels and metabolic activity was measured after 1, 3, and 7 days in culture using an alamarBlue assay. On both Soft and Stiff HA Nor-Tet hydrogels, there was a subtle increase in metabolic activity between one and three days, followed by a much larger increase between 3 and 7 days (FIG. 5E). While Soft and Stiff hydrogels support MSC adhesion and proliferation, Stiff HA Nor-Tet hydrogels displayed higher MSC densities and metabolic activity, which is due to stiffness-dependent differences in cellular mechanosensing

### Example 1-7: Matrix Stiffness Regulates MSC Morphology and Mechanosensing on RGD-Functionalized HA Nor-Tet Hydrogels

An important characteristic of stem cells is their ability to sense and respond to mechanical cues. For example, MSCs on soft hydrogels that mimic the stiffness of brain tissue express neural biomarkers, whereas MSCs cultured on more rigid hydrogels preferentially differentiate into cell types present in stiffer tissues, like muscle or bone. At the cellular level, changes in cell shape and spreading occur in response to mechanical cues shortly after making initial contact with their new environment. On a molecular level, cellular mechanosensing is led by several mechano-transducer proteins that collectively induce changes in focal adhesion maturation, cytoskeletal contractility, and nuclear Yes-associated protein (YAP) localization. To highlight the use of the self-forming HA Nor-Tet hydrogels presented as a facile platform for cellular mechanosensing studies, MSCs were cultured on Soft and Stiff RGD-functionalized hydrogels for three days and stiffness-dependent changes in morphology (area, roundness, aspect ratio) and mechanosensing (nuclear YAP localization, focal adhesion maturation, actin anisotropy) were evaluated.

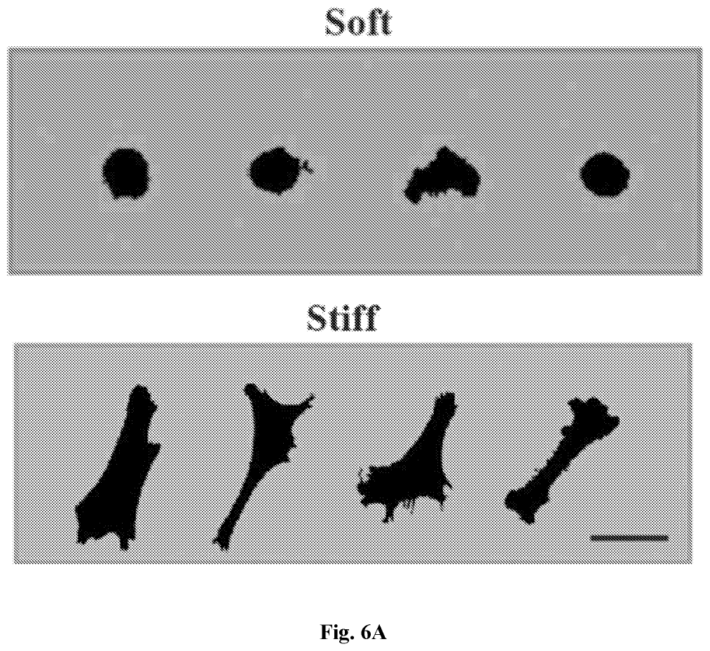

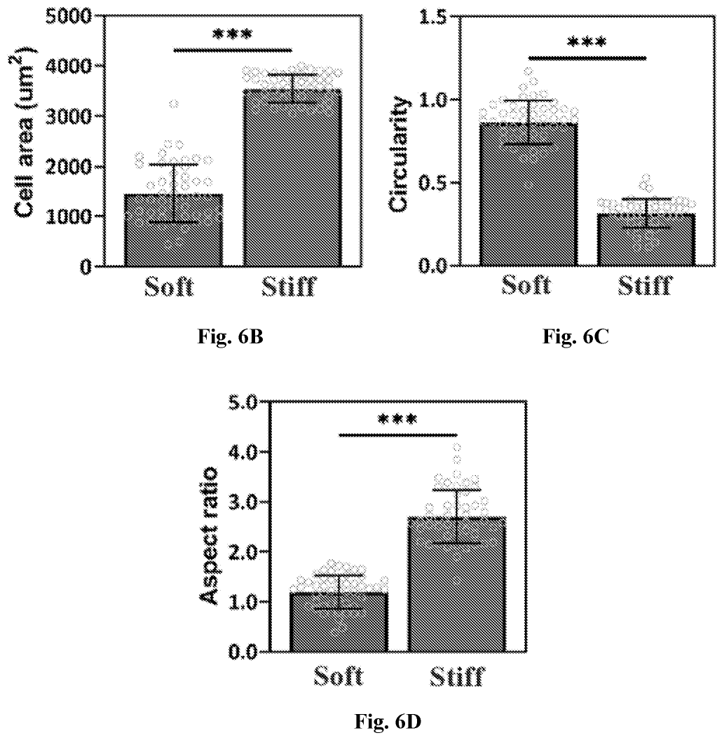

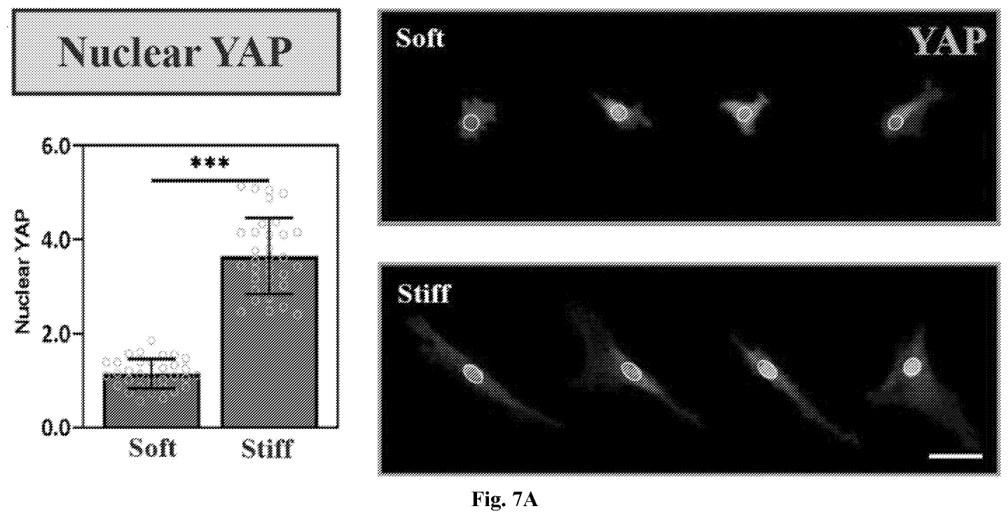

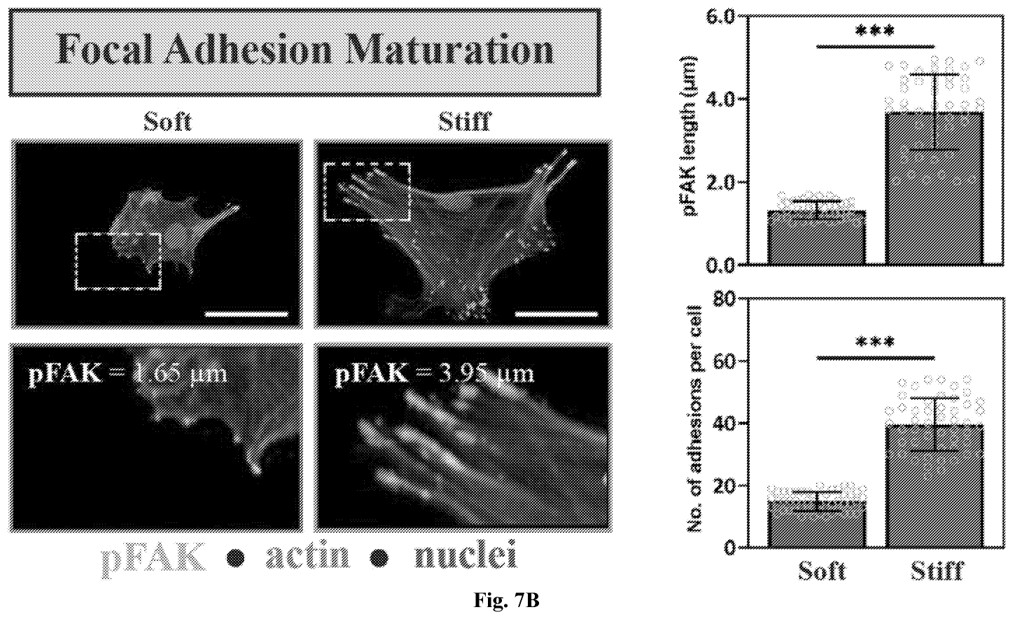

Representative silhouettes of MSCs on Soft and Stiff hydrogels show large differences in morphology induced by HA Nor-Tet hydrogel stiffness (FIG. 6A). MSCs on Soft HA Nor-Tet hydrogels were significantly smaller (1,500±600 μm²) than MSCs on Stiff HA Nor-Tet hydrogels (3,500±300 μm²) (FIG. 6B). MSCs were also significantly rounder on Soft HA Nor-Tet hydrogels (FIG. 6C) and had a larger aspect ratio on Stiff HA Nor-Tet hydrogels (FIG. 6D). These results are consistent with morphology of MSCs on photopolymerized HA hydrogels of comparable stiffness. For example, MSCs on softer (1 kPa) HANor hydrogels had an approximate average area of 650 μm², while on stiff (20 kPa) hydrogels MSC area increased to ˜3,750 μm². Similarly, on soft (˜3 kPa) o-Nitrobenzyl-Methacrylate-HA hydrogels MSCs had an average area of about 1,500 μm² while on stiff (˜15 kPa) hydrogels the area doubled to ˜3,300 μm². Although the HA Nor-Tet hydrogels were formed with a Diels-Alder click chemistry and not using free-radical polymerization between acrylamide and bis-acrylamide or photopolymerization reactions, stiffness-dependent changes in morphology were conserved.

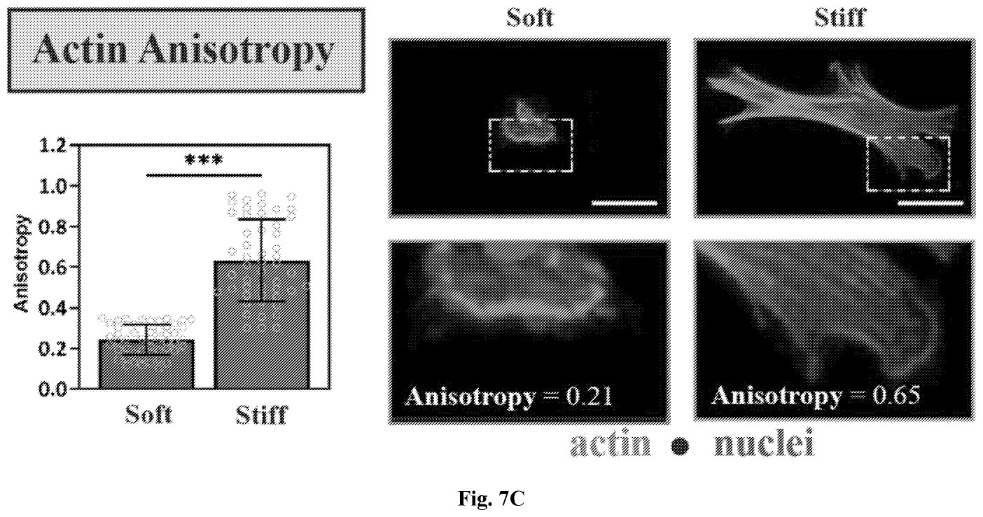

After establishing that MSC morphology changes in a stiffness-dependent manner on 2D Nor-Tet hydrogels, MSC mechanosensing on self-forming hydrogels was evaluated. Yes-associated protein (YAP) is a key transducer of outside-in mechanical signals, and cytosolic YAP translocates to MSC nuclei with increasing stiffness on polyacrylamide and photopolymerized 2D hydrogels. The nuclear YAP of MSCs on Stiff HA Nor-Tet hydrogels was significantly higher than MSCs on Soft HA Nor-Tet hydrogels (FIG. 7A). At the cell-hydrogel interface, focal adhesions act as intermediaries that connect the local extracellular environment to the actin cytoskeleton, consequently activating mechanotransductive pathways. Specifically, focal adhesion kinase (FAK) activation through the phosphorylation of tyrosine residues (pFAK) plays a role in cellular mechanosensing by responding to substrate stiffness and cytoskeletal contractility. As such, pFAK maturation (length, count) and cytoskeletal alignment (actin anisotropy) were used as additional proxies for stiffness-dependent mechanosensing.

The length of pFAK increased with increasing Nor-Tet hydrogel stiffness, and so did the average number of focal adhesions per cell (FIG. 7B). Actin anisotropy (actin fiber alignment) is a way to evaluate the organization of actin stress fibers, and there was a significant increase in actin anisotropy (0.24±0.07 vs. 0.63±0.20) between MSCs on Soft and Stiff Nor-Tet hydrogels (FIG. 7C). These results show that MSCs respond to mechanical cues on 2D Nor-Tet hydrogels which highlights the versatility of these catalyst-free hydrogels as a platform to study and control cellular mechanosensing.

### Example 1-8: Nor-Tet Hydrogels as a Delivery Vehicle of Highly Viable Cells

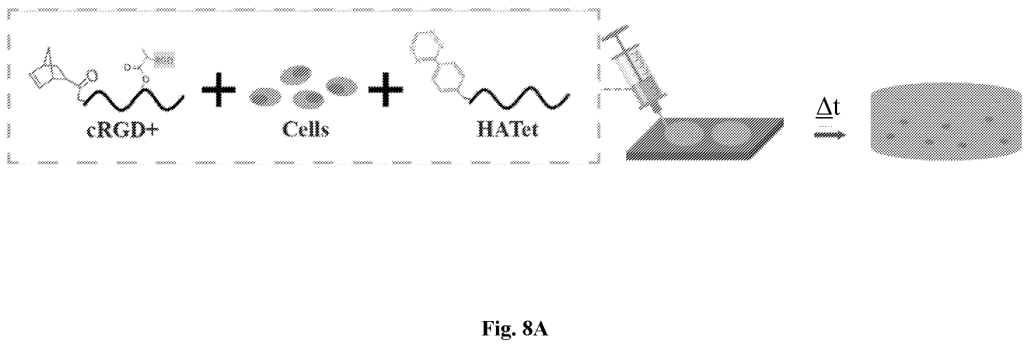

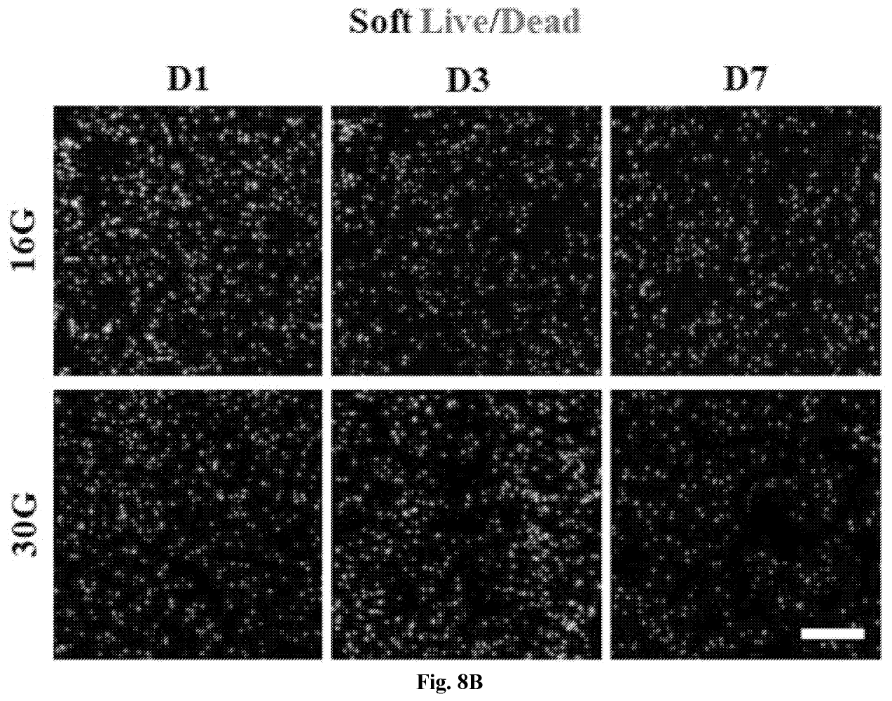

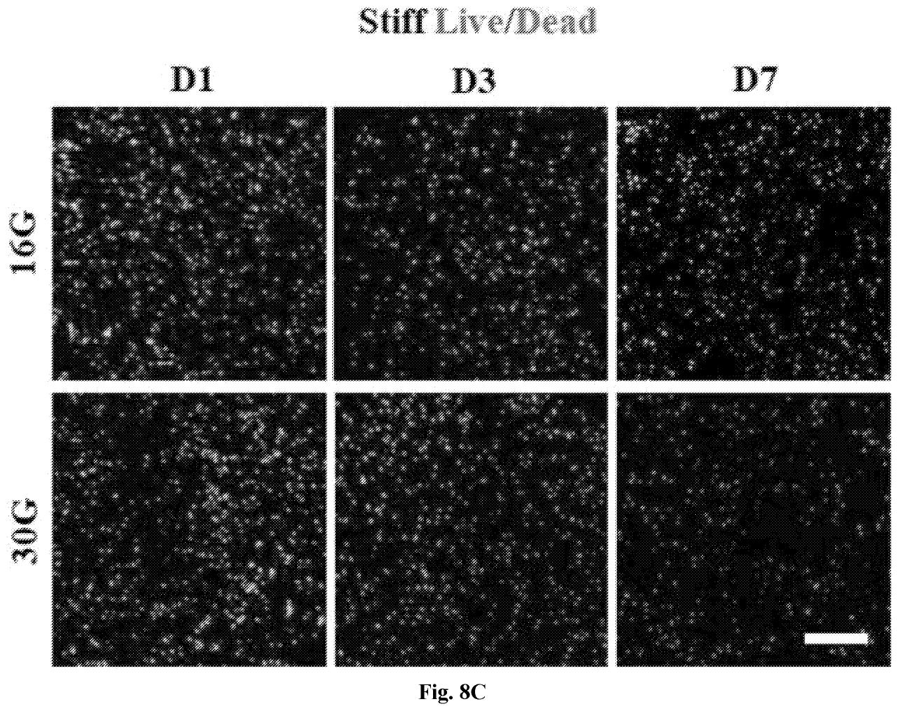

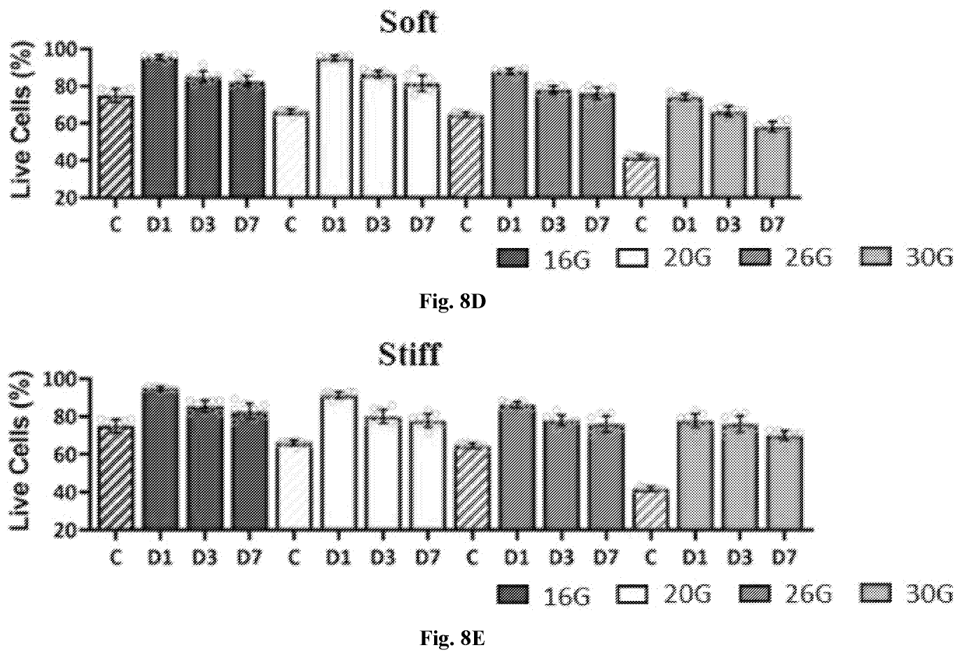

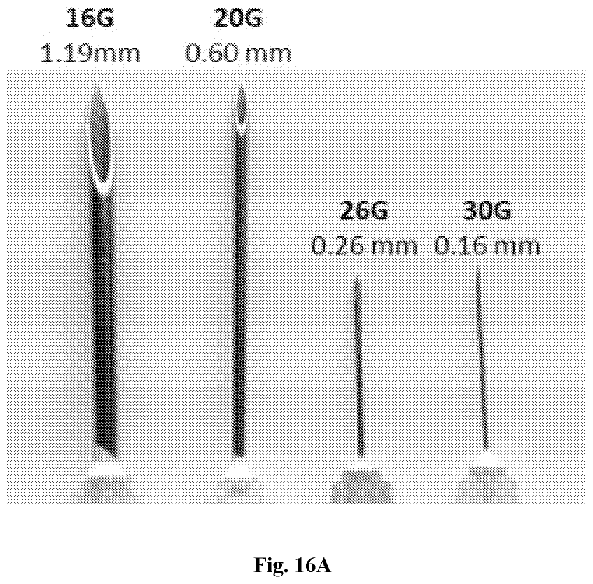

While cell injections are a widely used method to transplant cells, low cell viability and poor cell retention at the site of injection limit the efficacy of this minimally invasive technique. Hydrogels address these limitations by shielding cells from shear stresses during extrusion and by keeping cells within the hydrogel implant, and the use of hydrogels to deliver viable cells through a needle is typically done with softer hydrogels, since increasing mechanics has a negative impact on cell survival. To evaluate the use of HA Nor-Tet hydrogels as a biocompatible and injectable cell carrier, MSCs were suspended in culture medium or an RGD-functionalized HA Nor-Tet hydrogel solution that was loaded into a syringe and extruded through a needle tip onto cylindrical molds (FIG. 8A). Needles with varying inner diameters were used ranging from 160 μm (30 G) to 1,190 μm (16 G) (FIG. 16A). One, three-, and seven-days post-injection, the percentage of live cells extruded through different size needles was determined for Soft (2% w/v) and Stiff (6% w/v) HA Nor-Tet hydrogels by analyzing confocal images stained with a Live/Dead assay (live, green; dead, red) (FIGS. 8B-8C; FIGS. 16B-16C). One day post-injection, cells extruded through different size needles are highly viable (>85% viability: 16 G, 20 G, 26 G and >70% viability: 30 G) for Soft and Stiff groups (FIG. 8D-8E). Meanwhile, MSCs suspended in culture media alone have poor viability, with only 40%-70% cells viable one day post-extrusion. Seven days post-injection, MSCs in most groups remain viable (>80% viability: 16 G, 20 G and 55%-75% viability: 26 G, 30 G) for Soft and Stiff HA Nor-Tet hydrogels. The Soft and Stiff HA Nor-Tet hydrogels also retained their shape seven days post-injection, which demonstrates that viable MSCs can be extruded from a range of syringe needle sizes through a solution that self-forms into mechanically stable hydrogels.

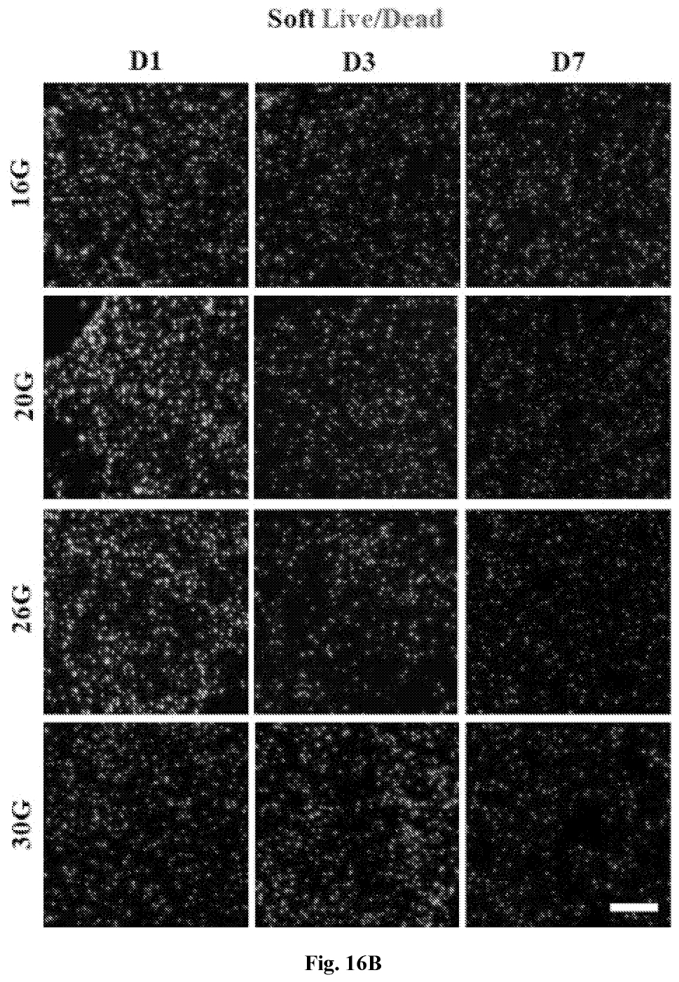

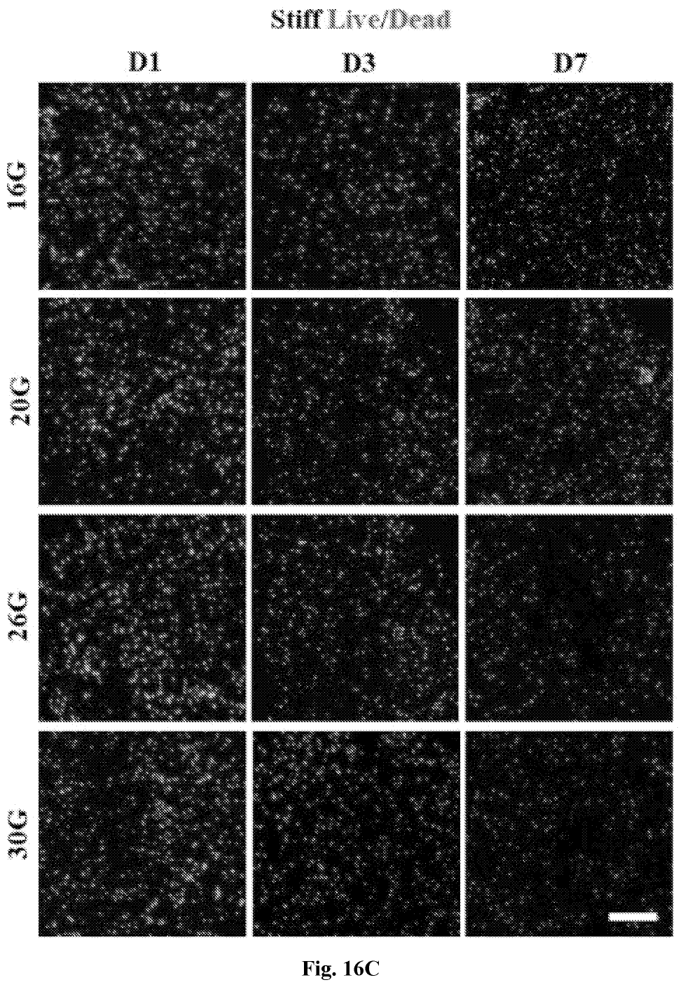

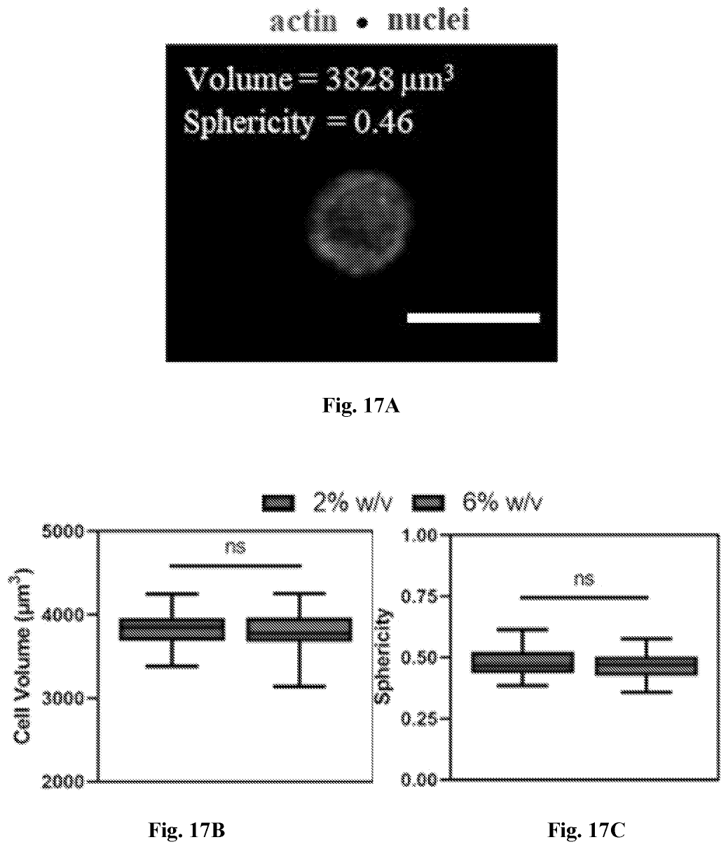

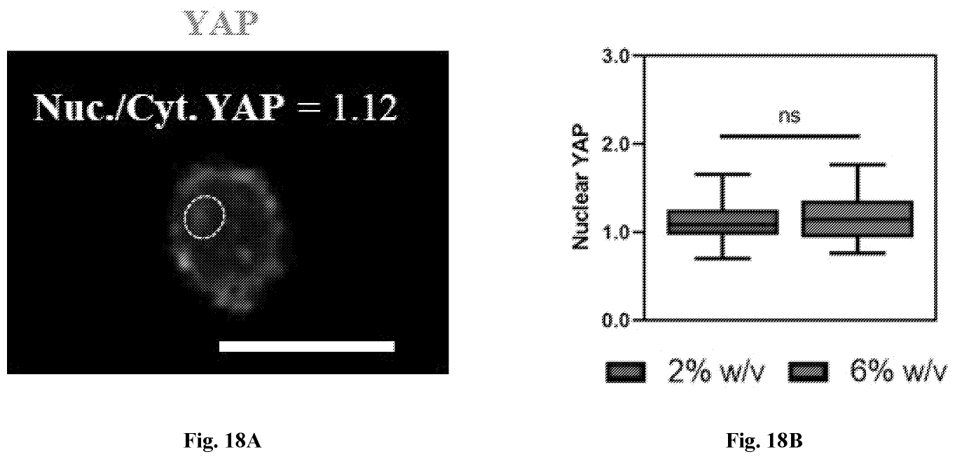

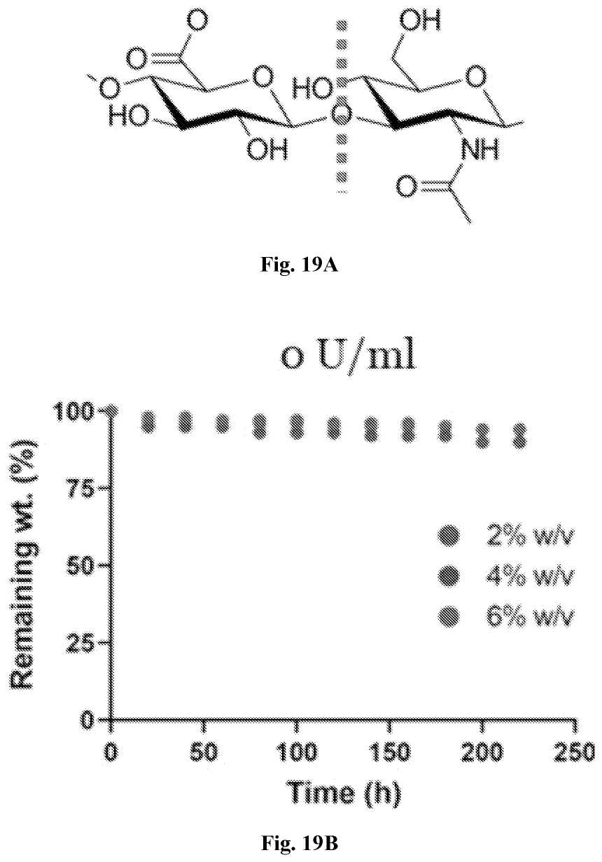

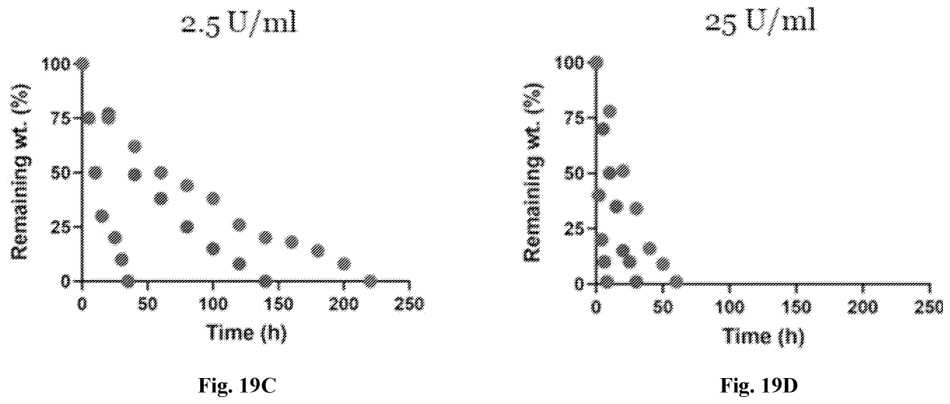

MSCs encapsulated in Soft and Stiff 3D HA Nor-Tet hydrogels remain spherical after 7 days in culture (FIG. 17A). Further, no difference in cellular volume (FIG. 17B) or sphericity (FIG. 17C) was observed between MSCs in 3D Soft and Stiff HA Nor-Tet hydrogels. These findings are consistent with previous studies showing that cells encapsulated in covalently crosslinked hydrogels remain spherical, independent of stiffness. As expected for spherical cells, nuclear YAP ratios were close to unity and did not change between Soft and Stiff Nor-Tet hydrogels (FIGS. 18A-18B). In contrast to HA hydrogels, gelatin hydrogels are permissive to cell remodeling which allows temporal cell spreading Koshy and colleagues encapsulated MSCs within soft hydrogels (G′ ˜350 Pa) formed with gelatin coupled with norbornene (GelNor) or tetrazine (GelTet). The MSCs in the gelatin hydrogels underwent extensive elongation, formed cell-cell contacts, and exhibited stress fibers. HA degrades in the presence of hyaluronidase, an enzyme physiologically present in the human body that cleaves the glycosidic bonds in the HA backbone. Indeed, Nor-Tet hydrogels degrade in the presence of hyaluronidase in a concentration-dependent manner. Thus, degradability of HA hydrogels by hyaluronidase to allow for 3D cellular remodeling can be investigated further (FIGS. 19A-19D).

### Example 1-9: 3D Cell-Mediated Nor-Tet Hydrogel Degradation Directs Stem Cell Differentiation

Stem cells can sense biophysical signals, leading to numerous cell functions including differentiation. On 2D substrates, MSC lineage commitment is dictated in part by stiffness, and this is due to stiffness-dependent changes in matrix mechanosensing. MSCs on 2D soft materials are round and exhibit low traction forces whereas MSCs on 2D stiff substrates are spread and more contractile. While MSCs spread with increasing stiffness on 2D, this is not the case in 3D. For 3D morphology-mediated MSC differentiation to occur, encapsulated MSCs need to locally remodel their surrounding hydrogel matrix, which can be enabled either by tuning hydrogel viscoelasticity or hydrogel degradation. To demonstrate the effects of cell-mediated hydrogel degradation on MSC differentiation, Khetan et al. encapsulated MSCs in hydrogels that restrict or permit 3D cellular spreading (Nat. Mater. 2013, 12, 458). In bipotential AD/OS (adipogenic/osteogenic) medium, MSCs spread and preferentially differentiate into osteoblasts in 3D enzymatically degradable hydrogels, whereas MSCs in 3D non-degradable hydrogels are round and commit to an adipogenic phenotype.

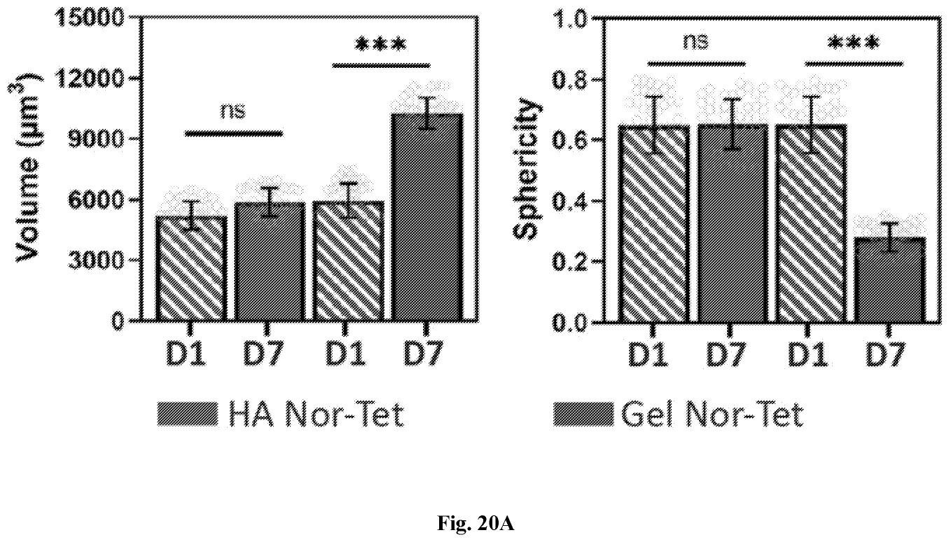

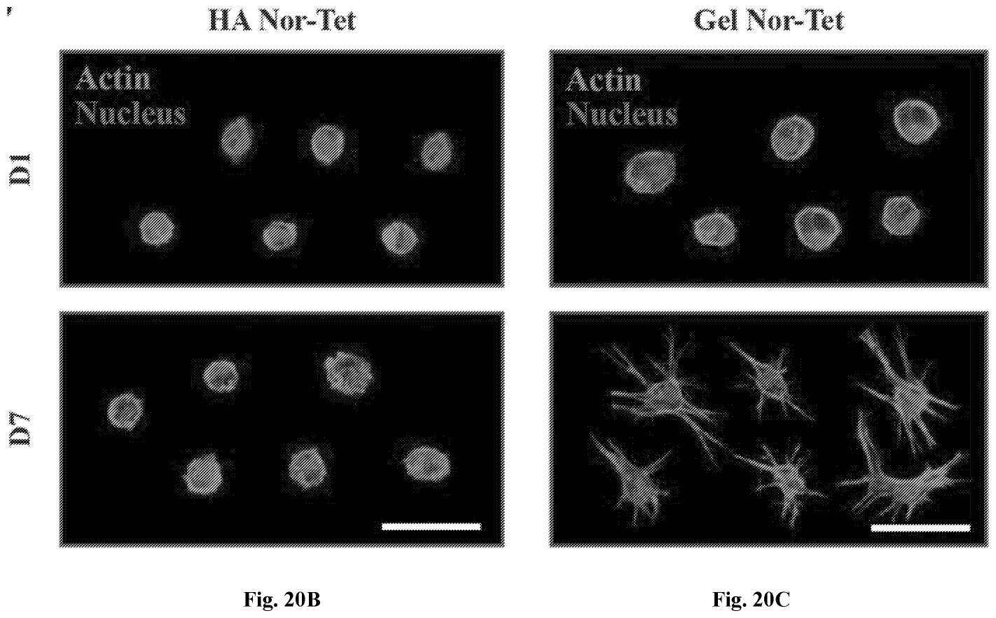

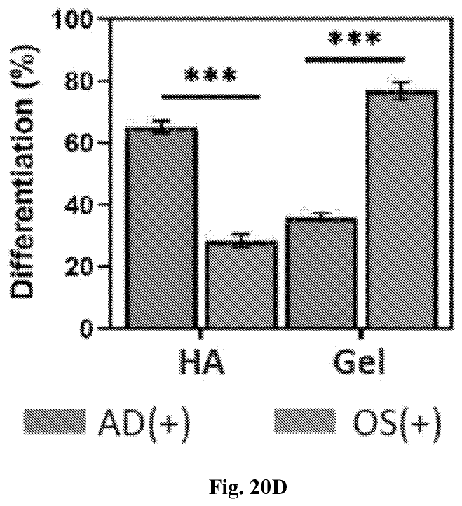

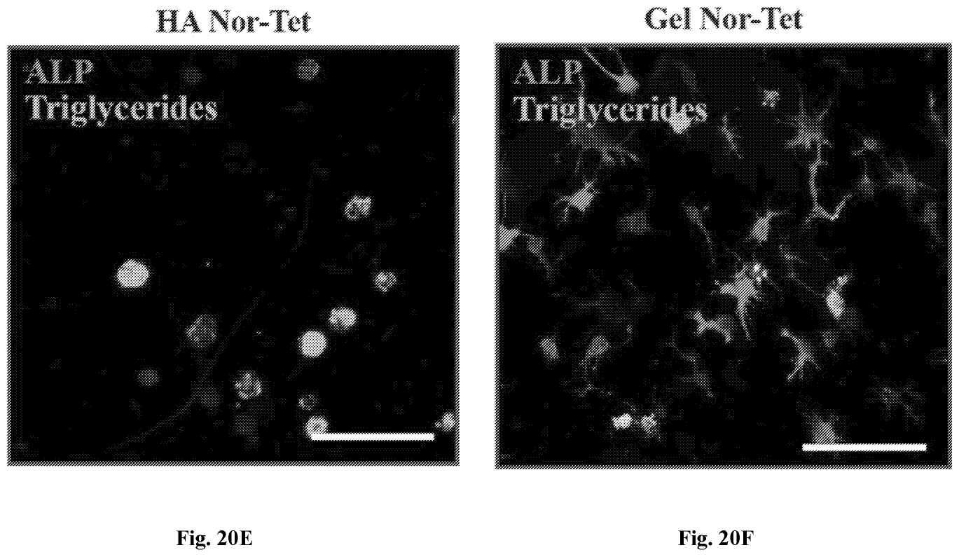

MSCs encapsulated in covalently crosslinked hydrogels can spread if the hydrogel contains protease-degradable components. Gelatin is enzymatically degradable and cells encapsulated in gelatin hydrogels spread, while MSCs encapsulated in HA hydrogels do not spread in vitro in the absence of hyaluronidase. Thus, the balance between stable HA and enzymatically degradable gelatin can be leveraged to modulate the extent of 3D spreading-mediated differentiation within injectable Nor-Tet hydrogels. Stable Nor-Tet hydrogels were formed by mixing HANor, HATet, and MSCs. Stiffness-matched (4.6 kPaE) degradable Nor-Tet hydrogels were formed by mixing enzymatically degradable GelNor with HATet and MSCs. One- and seven-days post-encapsulation, the morphology of MSCs (volume, sphericity) were determined for stable and degradable Nor-Tet groups. As expected, MSCs in stable Nor-Tet hydrogels remained small (volume 5198±700 μm³ on Day 1 and 5862±686 μm³ on Day 7) and round (sphericity 0.65±0.09 on Day 1 and 0.65±0.08 on Day 7) (FIG. 20A). MSCs in degradable Nor-Tet hydrogels had comparable volume (5947±849 μm³) and sphericity (0.65±0.09) after 1 day in culture, however, after 7 days there was an almost twofold increase in volume (10 275±758 μm³) and a significant drop in sphericity (0.27±0.05) (FIG. 20A). Representative images of MSCs in stable hydrogels show that they remain round for at least 7 days (FIG. 20B), whereas MSCs in degradable hydrogels are larger and display branching protrusions on Day 7 (FIG. 20C). These findings show that 3D cellular spreading in Nor-Tet hydrogels can be induced simply by substituting HANor with enzymatically degradable GelNor macromer. After demonstrating that encapsulated MSCs in Nor-Tet hydrogels can take on spread or round morphologies depending on the polymer backbone used, the effects of 3D cellular spreading on MSC lineage commitment were investigated. MSCs were encapsulated in stable or degradable Nor-Tet hydrogels, cultured in AD/OS Medium for seven days, and co-stained with alkaline phosphatase (ALP, OS biomarker) and intracellular triglycerides (AD biomarker) to identify OS(+) and AD(+) cells, respectively. After seven days in culture, 65±2% of cells in stable HA hydrogels are AD(+) which is over a 1.8-fold increase compared to cells in degradable gelatin hydrogels (36±1%) (FIG. 20D). In contrast, only 28±2% of cells in stable HA hydrogels are OS(+) while 77±3% of cells in degradable gelatin hydrogels are OS(+) (FIG. 20D). Representative image of cells in stable HA Nor-Tet hydrogels co-stained with ALP and lipid droplets show that cells retain a spherical morphology and most cells are AD(+) (FIG. 20E). In contrast, representative image of cells in degradable gel Nor-Tet hydrogels are highly spread and most cells display intracellular ALP (FIG. 20F). These findings show that the Nor-Tet hydrogels presented are biocompatible and can regulate 3D cellular spreading and downstream differentiation by incorporating enzymatically degradable GelNor in lieu of HANor.

### Example 1-10

In the present study, HA macromers with a high degree of Nor and Tet substitution were developed to synthesize HA Nor-Tet hydrogels that form without an external catalyst. These hydrogels are mechanically stable, and gelation times and stiffness are tunable by changing the total HA macromer concentration and stoichiometric ratio between HANor and HATet macromers. The bioactivity of HA Nor-Tet hydrogels was independently controlled by covalently tethering peptides to Me groups in methacrylated HANor macromers. MSCs on 2D RGD-functionalized HA Nor-Tet hydrogels adhere, proliferate, and are mechanically sensitive to changes in hydrogel stiffness. These hydrogels also support 3D encapsulation, and MSCs in HA Nor-Tet hydrogel precursor solution are highly viable and protected from shear forces experienced while extruding from syringe needles during cellular injections. The HA Nor-Tet hydrogels presented here can be independently tuned and have broad applicability in basic and translational research.

Also, in the present study, biocompatible Nor-Tet hydrogels are developed with tunable physical and biochemical properties. Mechanical parameters of these self-forming hydrogels are controlled by varying macromer concentration and stoichiometric ratios between Nor and Tet moieties. By modifying Nor containing macromers with thiolated peptides, bioactivity is incorporated independent of stiffness or gelation time. MSCs on 2D RGD-functionalized Nor-Tet hydrogels adhere, proliferate, and are mechanically sensitive to changes in hydrogel stiffness. MSCs in 3D Nor-Tet hydrogel solutions are more viable than MSCs injected in liquid, and 3D cellular spreading-mediated differentiation is controlled by simply substituting stable HANor with enzymatically degradable GelNor. These self-forming Nor-Tet hydrogels feature a wide range of physicochemical properties and have broad applicability in fundamental and translational research.

### Example 1-11: Materials and Methods

### HANor and HATet Macromer Synthesis:

dry HATBA) in To synthesize HANor, carboxyl groups in HA were modified with norbornene (Nor) as previously described (Vega et al., Nat. Commun. 2018, 9, 614). Briefly, sodium hyaluronate (NaHA, Lifecore, 60 kDa) was converted to its tetrabutylammonium salt (HATBA) by dissolving in distilled water (2% w/v) and mixing with Dowex resin for two hours at room temperature. The resin was then vacuum filtered, and the pH was adjusted to 7.02 using tetrabutylammonium hydroxide (TBA-OH) diluted in water (1:1 v/v). The resulting HATBA solution was then frozen and lyophilized. Carboxyl groups in HATBA were then modified with Nor via amidation with 5-norbornene-2-methylamine (Nor-NH₂, 0.4 mmol per gram of anhydrous dimethyl sulfoxide (DMSO, 2% w/v) and benzotriazole-1-yl-oxy-tris-(dimethylamino)-phosphonium hexafluorophosphate (BOP) under nitrogen for two hours at room temperature. The reaction was quenched with cold distilled water, dialyzed (SpectraPor, 6-8 kDa molecular weight cutoff), frozen, and lyophilized. The synthesized HANor macromer had ˜50% of its repeat units functionalized with Nor, as analyzed with 1H NMR spectroscopy (FIG. 11A). The percentage of modification was calculated by comparing the integral of the methyl HA peaks between δ 1.8-2.0 ppm to the vinyl proton peaks of norbornene between δ 6.2-6.3 ppm.

To synthesize HATet macromers, carboxyl groups in HA were modified with tetrazine (Tet) using a modified procedure described by Desai and coworkers (Desai et al., undefined 2015, 50, 30). Briefly, HATBA was dissolved (1% w/v) in 100 mM B—(N-morpholino) ethanesulfonic acid (MES) buffer (pH 6) and 1-(3-dimethylaminopropyl)-3-ethylcarbodiimide (EDC), N-hydroxysuccinimide (NHS), and tetrazine-amine (Tet-NH₂) were added at a 1:4:1 molar ratio at 0.5 mmol Tet per gram of HATBA and reacted overnight at room temperature. The HATet solution was then dialyzed (SpectraPor, 6-8 kDa molecular weight cutoff), frozen, and lyophilized. 1H NMR spectroscopy analysis confirmed that ˜40% of HATet repeat units were functionalized with Tet (FIG. 11B). The percentage of modification was calculated by comparing the integral of the methyl HA peaks between δ 1.8-2.0 ppm to the aromatic proton peak of tetrazine between 810.4 ppm (Koshy et al., Adv. Healthc. Mater. 2016, 5, 541).

### GelNor Macromer Synthesis:

To synthesize GelNor macromers, carboxyl groups in gelatin were modified with Nor using a procedure previously described by Koshy and coworkers (Adv. Healthcare Mater. 2016, 5, 541). Briefly, gelatin (TypeA, 300 bloom) was dissolved (1% w/v) in 100 mm MES buffer (pH 6) and EDC, NHS, and Nor-NH₂ were added at a 2:1:1 molar ratio at 2 mmol Nor-NH₂ per gram of gelatin and reacted for 4 h at 37° C. The Gel Nor solution was then dialyzed (Spectra Por, 6-8 kDa molecular weight cutoff), frozen, and lyophilized. 1H NMR spectroscopy analysis confirmed that ˜15% of Gel Nor repeat units were functionalized with Nor. The percentage of modification was calculated by comparing the integral of the aromatic amino acid peaks between δ 7.0-7.5 ppm to the vinyl proton peaks of norbornene between& 6.0-6.5 ppm.

HANor (cRGD+) Synthesis:

To synthesize HANor (cRGD+) macromers, hydroxyl groups in HANor were first modified with methacrylates (Me) to form HANorMe via esterification with methacrylic anhydride (MA) by adapting a previously described protocol (Smeds et al, 2000, DOI 10.1002/1097-4636). HANor was dissolved (1% w/v) in distilled water at 4° C. A 15-fold molar excess of MA was added dropwise while maintaining pH between 8.5-9.0. After all MA was added, the solution was left stirring overnight at room temperature. The HANorMe solution was then dialyzed (SpectraPor, 6-8 kDa molecular weight cutoff), frozen, and lyophilized. 1H NMR spectroscopy analysis confirmed that ˜60% of HANor repeat units were functionalized with Me (FIG. 3C). The percentage of modification was calculated by comparing the integral of the HA peaks between δ 1.8-2.0 ppm to the alkene proton peaks of methacrylates between δ 5.5-6.5 ppm.

To peptide-functionalize HANorMe, thiols in cysteine-containing peptides were coupled to Me groups in HANorMe via an aqueous Michael addition reaction. Briefly, HANorMe was dissolved (1% w/v) in 200 mM triethanolamine (TEOA) buffer (pH 8) at room temperature. Thiolated RGD peptide (sequence: GCGYGRGDSPG, cRGD) in solution (50 mM in PBS) was added dropwise to reach a final cRGD concentration that corresponds to 2 mM in self-forming hydrogels of varying weight percent (2%, 4%, or 6% final w/v). Table 1-3 summarizes the reaction parameters to create HA Nor (cRGD+) macromers with appropriate cRGD concentrations. The HANor (cRGD+) solution was then dialyzed (SpectraPor, 6-8 kDa molecular weight cutoff), frozen, and lyophilized. 1H NMR spectroscopy analysis confirmed that 17%, 9%, and 6% of Me groups were coupled with cRGD for 2%, 4%, and 6% w/v HANor (cRGD+) macromers (FIGS. 15A-15C). The percentage of Me-cRGD coupling was calculated by comparing integral of the alkene proton peaks of methacrylate between δ 5.5-6.5 ppm to the B and y ethyl protons of the arginine moiety between δ 1.50-2.00 ppm.

### Preparation and Characterization of Self-Forming Hydrogels:

Rheological properties of self-forming hydrogels were measured using a Discovery Hybrid Rheometer (DHR-3, TA Instruments) with a 20 mm diameter 1° cone upper plate geometry and the lower plate was heated to a physiological temperature of 37° C. Samples were prepared by dissolving HANor and HATet separately in PBS, followed by mixing, resulting in 2%, 4%, or 6% final w/v. Immediately after mixing, 40 μl of the solution was pipetted to the center of the rheometer stage and the shear storage (G′) and loss (G″) moduli were monitored. To confirm that click crosslinking between Nor and Tet moieties creates stable hydrogels, frequency sweeps were performed at 1% oscillatory strain while varying the frequency from 0.01 to 10 Hz. To measure plateau G′ and gelation kinetics, time sweeps were performed for 1 hour at 1 Hz and 1% oscillatory strain. Bulk mechanical properties of self-forming hydrogels were measured using a Shimadzu EZ-SX Mechanical Tester equipped with a 50 N compression load. Briefly, hydrated HANor and HATet were mixed and pipetted into cylindrical silicone molds (2 mm height, 8 mm diameter). The hydrogels were allowed to form for 30 minutes before soaking in PBS overnight at 37° C. Formed cylindrical Nor-Tet hydrogels were compressed until 30% strain, and the elastic modulus was determined using the slope of the stress-strain curve between 10% and 20% strain.

### 2D Cell Attachment and Proliferation on Self-Forming Hydrogels:

Thin hydrogels were formed by dissolving HANor (with or without cRGD functionalization) and HATet separately in Growth Medium (α-Minimum Essential Medium (αMEM) supplemented with 10% fetal bovine serum (FBS, Lonza)), followed by mixing, resulting in Soft (2% w/v) and Stiff (6% w/v) hydrogel solutions. Immediately after mixing, 70 μl of the solution was pipetted into cylindrical polydimethylsiloxane (PDMS) molds (11 mm diameter, 1 mm height). The thin hydrogels were allowed to crosslink for 2 mins followed by gently adding Growth Medium to prevent hydrogels from drying out as they continue to polymerize. To evaluate cell adhesion and proliferation, human primary MSCs (from bone marrow, Lonza) were expanded in 100 mm petri dishes in Growth Medium. MSCs were seeded on top of 2D Nor-Tet hydrogels at a density of 3,000 cells per cm². To evaluate cell attachment, cells were fixed after 3 days in culture using 10% neutral buffered formalin for 10 mins at room temperature. Samples were washed twice with PBS after fixation and kept at 4° C. until immunostaining was performed. To evaluate cell proliferation, culture media was removed and replaced with alamarBlue working solution (Growth Medium supplemented with 10% alamarBlue Reagent, Invitrogen) after 1, 3, and 7 days in culture, and kept in the cell incubator for 4 h. Samples of the media were collected (50 μl in triplicate) and fluorescence was measured with a plate reader (560 nm excitation, 590 nm emission).

### 3D Cell Encapsulation in Self-Forming Hydrogels:

For cell encapsulation, cells were resuspended in RGD-functionalized HANor dissolved in Growth Medium at a density of 10⁶ cells per mL. HATet dissolved in Growth Medium was mixed, resulting in a 2% w/v hydrogel solution, which was injected into a PDMS mold (8 mm diameter, 2 mm height). The hydrogels were allowed to crosslink for 2 min followed by gently adding Growth Medium to prevent hydrogels from drying out as they continue to polymerize. To evaluate cell viability, culture media was removed and replaced with Live/Dead Viability working solution (Growth Medium supplemented with 1:1,000 calcein AM and 1:1,000 ethidium homodimer, Invitrogen) and kept in a cell incubator for 30 mins prior to confocal imaging and imaging-based analysis.

### Immunostaining and Imaging-Based Analysis:

After 3 days in culture, MSC-laden hydrogels were fixed in 10% formalin for 10 min at room temperature. Samples were then permeabilized with 0.1% Triton X-100 for 2 mins and blocked with 3% bovine serum albumin in PBS for 30 mins. Primary YAP or pFAK antibody (Santa Cruz Biotechnologies, 1:200) was added for 1 h, followed by Alexa Fluor 488 secondary antibody (Life Technologies, 1:200) for 2 h at room temperature. To visualize individual cells and nuclei, samples were stained for actin (Alexa Flour 568 phalloidin, 20 mins, 1:100) and double stranded DNA (Hoechst, 5 mins, 1:1,000), respectively. Confocal imaging was performed with a Nikon A1 confocal microscope.

Imaging-based cell analysis was performed using ImageJ software (National Institutes of Health). For 2D morphology analysis, the wand tracing tool was used to select cell outlines from the actin channel and the measure function was used to calculate cell area, circularity, and aspect ratio. For 2D YAP analysis, nuclear YAP localization was determined by first measuring the integrated density of YAP of five regions of interest (ROI) on the cytoplasm and nucleus of each cell, respectively. The ratio between the nuclear and cytosolic integrated densities was defined as the nuclear YAP value. To evaluate focal adhesion maturation, the number of pFAK adhesions per cell were counted using the Find Maxima feature, and at least 10 focal adhesion lengths were measured per cell. Actin anisotropy was quantified by determining the common directionality of actin fibers within a manually-defined ROIs (at least three ROIs surrounding the cell nucleus using the FibrilTool plugin (Boudaoud et al., Nat. Protoc. 2014, 9, 457).

For 3D morphology analysis, z-stacks of the actin channel were binarized using the Otsu thresholding method. Cell volume and surface area were determined using the 3D Objects Counter feature, and these values were used to calculate sphericity as previously reported (Caliari et al., Biomaterials 2016, 103, 314). For 3D YAP analysis, actin cytoskeleton and nucleus z-stacks were binarized using the Otsu thresholding method. These cellular and nuclear ROIs were then superimposed with the YAP channel to obtain 3D YAP stacks of the cytoplasmic and nuclear space. The integrated density of YAP was then quantified using the 3D Objects Counter feature and the ratio between the nuclear and cytoplasmic YAP intensity was reported as the nuclear YAP ratio.

### MSC Syringe Needle Flow and Encapsulation Viability Study:

MSCs were resuspended at a cell density of 10⁶ cells per mL in either Growth Medium or an RGD-functionalized HA Nor-Tet hydrogel solution. The suspensions were loaded into a 1 mL syringe with an appropriate gauge needle and ejected onto a sterile glass coverslip (for cells suspended in Growth Medium) or into PDMS molds (for cells suspended in hydrogel solution) at a volumetric flow rate of 3,000 μL per min. The hydrogels were allowed to crosslink for 2 min followed mins followed by gently adding Growth Medium to prevent hydrogels from drying out as they continue to polymerize. To evaluate cell viability, culture media was removed and replaced with Live/Dead Viability working solution (Growth Medium supplemented with 1:1,000 Calcein AM and 1:1,000 ethidium homodimer, Invitrogen) and kept in a cell incubator for 30 mins prior to confocal imaging and imaging-based analysis.

### MSC Differentiation Study:

MSCs were suspended in RGD-functionalized HANor (stable) or GelNor (degradable) dissolved in growth medium at a density of 10⁶ cells/mL. To maintain GelNor in liquid phase, GelNor solutions were maintained at 37° C. prior to and during mixing with HATet. HATet dissolved in growth medium was then mixed with HANor or GelNor solution (2% w/v total macromer concentration), which was injected into a PDMS mold (8 mm diameter, 2 mm height). The hydrogels were allowed to crosslink for 2 min followed by washes in growth medium. MSC-laden hydrogels were cultured in either growth medium, OS medium (hMSC Osteogenic Differentiation BulletKit Medium, Lonza), ADMedium (hMSC Adipogenic Differentiation BulletKit Medium, Lonza) or bipotential AD/OSMedium (1:1 OSMedium and AD Medium) for 7 days. To evaluate cell differentiation, cell-laden hydrogels were fixed with 10% neutral buffered formalin for 30 min. Fixed samples were co-stained with BODIPY (1:2500 dilution) to visualize triglycerides and Vector Blue Alkaline Phosphatase Substrate prepared according to manufacturer specifications to visualize ALP. Samples were counterstained with Hoescht to visualize nuclei, and the percentage of OS(+) and AD(+) cells was determined by counting the number of cells that stained positive for ALP and BODIPY, respectively.

### Statistics:

The statistical analysis was performed using GraphPad Prism 9.3.1 software. All experiments were carried out in triplicates and single cell analysis was done with at least 50 cells per group. All graphs represent mean±standard deviation (SD). For comparisons of three or more groups: normally distributed populations were analyzed via analysis of variance (ANOVA) with a Tukey's post hoc test to correct for multiple comparisons. Differences among groups are stated as p<0.05 (*), p<0.01 (**), p<0.001 (***), and stated as (ns) when differences between groups are not statistically significant.

### Example 2-1

Osteoporosis is a disease characterized by a decrease in bone mineral density, thereby increasing the risk of sustaining a fragility fracture. Most medical therapies are systemic and do not restore bone in areas of need, leading to undesirable side effects. Injectable hydrogels can locally deliver therapeutics with spatial precision, and this study reports the development of an injectable hydrogel containing a peptide mimic of bone morphogenetic protein-2 (BMP-2). To create injectable hydrogels, hyaluronic acid was modified with norbornene (HANor) or tetrazine (HATet) which upon mixing click into covalently crosslinked Nor-Tet hydrogels. By modifying HANor macromers with methacrylates, thiolated BMP-2 mimetic peptides were immobilized to HANor via a Michael addition reaction, and coupling was confirmed with 1H NMR spectroscopy. BMP-2 peptides presented in soluble and immobilized form increased alkaline phosphatase (ALP) expression in MSCs cultured on 2D and encapsulated in 3D Nor-Tet hydrogels. Injection of bioactive Nor-Tet hydrogels into hollow intramedullary canals of Lewis rat femurs showed a local increase in trabecular bone density as determined by micro-CT imaging. The presented work shows that injectable hydrogels with immobilized BMP-2 peptides are a promising biomaterial for the local regeneration of bone tissue and for the potential local treatment of osteoporosis.

### Example 2-2

Osteoporosis is characterized by a reduction in bone mineral density and disruption of bone microarchitecture. Osteoporosis is the most common chronic metabolic bone disease with an estimated 200 million people affected worldwide. According to the International Osteoporosis Foundation, 1 in 3 women above the age of 50 and 1 in every 5 men will experience fragility fractures resulting from osteoporosis in their lifetime. Osteoporosis increases the chances of fragility fractures after a low-level fall, with a mortality rate of up to 69% within ten years. Risk factors for osteoporosis include genetics, being of postmenopausal age, substance abuse, poor dietary intake, and inactivity. These risk factors disrupt bone remodeling, a dynamic physiological process in which bone tissue is resorbed by osteoclasts and formed by osteoblasts. In osteoporosis, the rate of bone resorption is greater than the rate of bone formation, especially in major weight-bearing bones including vertebrae in the lumbar spine and femurs. In the early stages of osteoporosis, bone loss is mainly observed in trabecular or cancellous bone, made up of a trabeculae network with high bone turnover. Current treatments for osteoporosis are antiresorptive and anabolic drugs which can cause systemic side effects including oncogenesis. Hormonal therapies are used as a last resort and only prescribed to high-risk post-menopausal women because it can cause adverse side effects including blood clots.

Efforts to develop alternative strategies to regenerate bone have utilized recombinant human bone morphogenetic protein 2 (BMP-2), a potent inducer of osteogenesis in vivo. The osteogenic signaling cascade begins when BMP-2 binds to BMP-receptor type II, which leads to the phosphorylation (activation) of BMP-receptor type I, and ultimately to the phosphorylation of Smad1, a cytoplasmic signaling molecule for BMP-2. Phosphorylated Smad1 then localizes to the nucleus and controls gene expression to initiate osteogenic differentiation. In vitro, embryonic stem cells, human mesenchymal stem cells (MSCs), and C2C12 myoblasts exposed to BMP-2 express increased levels of cytoplasmic alkaline phosphatase (ALP), a well-established biomarker of osteogenesis. Despite its osteoinductive properties, the clinical use of BMP-2 is limited by unwanted side effects. For instance, collagen scaffolds with high doses of untethered BMP-2 used for spinal fusions have resulted in serious complications including ectopic bone formation and impaired function.

BMP-2 is a large and expensive growth factor, and BMP-2 based therapies are associated with inconsistent outcomes and safety concerns. Peptides that mimic the bioactivity of native BMP-2 are an inexpensive and potentially more efficacious substitute that can be incorporated into biomaterials. Specifically, the DWIVA peptide sequence from BMP-2 has high receptor-binding activity and specificity to BMP-receptor types I and II, and this sequence has been tested for enhancing osteogenic differentiation and bone formation. It was found that osteoblast-like MC3T3-E1 cells cultured on titanium (Ti) chemically modified with DWIVA peptides had higher levels of alkaline phosphatase (ALP), and DWIVA-treated Ti dental implants induced increased bone formation in vivo. Mesenchymal stem cells (MSCs) encapsulated in DWIVA-functionalized self-assembling nanofibrous hydrogel networks and alginate hydrogels also commit to osteogenic lineages, as evidenced by increased ALP activity and mineralization.

Hyaluronic acid (HA) is an abundant extracellular matrix component that mediates cellular signaling, matrix organization, and morphogenesis. HA polymers are amenable to chemical modifications through carboxyl and hydroxyl functional groups that can be used as macromers to create highly tunable hydrogels via various polymerization schemes. For instance, Diels-Alder reactions between macromers modified with dienes (e.g., norbornene, Nor) and dienophiles (e.g., tetrazine, Tet) yield self-forming hydrogels that can be injected. In the present study, it was hypothesized that HA modified with Nor or Tet moieties can be used to create injectable DWIVA-functionalized Nor-Tet hydrogels that enhance osteogenic differentiation of MSCs in vitro and induce trabecular bone growth in vivo. To test this hypothesis, the present study developed an injectable HA hydrogel system by modifying HA with Nor (HA Nor) or Tet (HATet) moieties. BMP-2 signals were immobilized in the hydrogels by methacrylating HANor macromers (HANorMe) and pre-coupling Me-groups with thiolated DWIVA peptides via an aqueous Michael addition reaction. Osteogenic differentiation of MSCs cultured atop or within DWIVA-functionalized hydrogels were investigated by quantifying ALP via fluorescent imaging-based analysis. New trabecular bone formation in rat femurs injected with HA Nor-Tet hydrogels with or without DWIVA peptide was also evaluated using micro-computed tomography (micro-CT).

### Example 2-3: Materials and Methods

Sodium hyaluronate (NaHA, 60 kDa, HA60K-5) was purchased from Lifecore Biomedical (Chaska, MN). Dowex® resin 50WX2 hydrogen form 100-200 mesh, methacrylic anhydride (MA), triethanolamine (TEOA), and Fast Blue RR Assay (fast blue) were purchased from Sigma Aldrich (Burlington, MA). Dimethyl sulfoxide (DMSO), benzotriazole-1-yl-oxy-tris-(dimethylamino)-phosphonium hexafluorophosphate (BOP), B—(N-morpholino) ethanesulfonic acid (MES), and SpectraPor 6-8 kDa molecular weight cutoff dialysis tubing were purchased from Millipore Sigma (St. Louis, MO). Tetrabutylammonium hydroxide (TBA-OH) was purchased from Acros Organics (Geel, Belgium). 5-norbornene-2-methylamine (Nor-NH₂) and N-hydroxysuccinimide (NHS) were purchased from TCI America (Portland, OR). 1-(3-dimethylaminopropyl)-3-ethylcarbodiimide (EDC) and Hoechst were purchased from Thermo Scientific (Waltham, MA). Tetrazine-amine (Tet-NH₂) was purchased from Kerafast (Boston, MA). Thiol-containing peptide mimic of BMP-2 with sequence GCGGGDWIVAG (SEQ ID NO:8) (DWIVA) was purchased from GenScript (Piscataway, NJ). Human mesenchymal stem cells (MSCs) from bone marrow and Osteogenic Differentiation BulletKit™ Medium (OS Medium) were purchased from Lonza (Walkersville, MD). Minimum essential medium alpha (MEM-α) with no nucleosides, penicillin/streptomycin (10,000 U/ml), and fetal bovine serum (FBS) were purchased from Gibco (Waltham, MA). Silicone elastomer, Sylgard™ 184 was purchased from Electron Microscopy Sciences (Hatfield, PA). Isoflurane solution and 70% isopropyl alcohol were purchased from Covetrus (Portland, ME). Buprenorphine SR 1 mg/mL and meloxicam SR 2 mg/mL were purchased from ZooPharm (Laramie, WY). 2% chlorohexidine was purchased through Covetrus (Portland, ME) and supplied by Vedco (Saint Joseph, MO).

### Hydrogel Macromer Synthesis and Characterization

### HANor

Carboxyl groups in HA were modified with norbornene (Nor) as previously described (Vega et al., Nat. Commum. 9, 614, 2018). Briefly, NaHA was converted to its tetrabutylammonium salt (HATBA) by dissolving in distilled water (2% w/v) and mixing with Dowex resin (3 g resin per 1 g NaHA) for two hours at room temperature. The resin was then vacuum filtered, and the pH was adjusted to 7.02 using TBA-OH diluted in water (1:1 v/v). The resulting HATBA solution was frozen and lyophilized. Carboxyl groups in HATBA were then modified with Nor via amidation with Nor-NH₂ (0.4 mmol per 1 g of HATBA) in anhydrous DMSO (0.5 ml per 0.1 g HATBA) and BOP (0.38 g per 1 g HATBA) under nitrogen for two hours at room temperature. The reaction was quenched with cold distilled water, dialyzed with SpectraPor dialysis tubing, frozen, and lyophilized. The synthesized HANor macromer had ˜50% of its repeat units functionalized with Nor, as analyzed with ¹H NMR spectroscopy. The percentage of modification was calculated by comparing the integral of the methyl HA peaks between δ 1.8 to 2.0 ppm to the vinyl proton peaks of norbornene between δ 6.2 to 6.3 ppm (Miao et al., Polym. Chem. 2015).

### HATet

Carboxyl groups in HA were modified with tetrazine (Tet) using a modified procedure previously described (Desai et al., Biomaterials 50, 30-37. 2015). Briefly, HATBA was dissolved (1% w/v) in 100 mM MES buffer (pH 6). EDC, NHS, and Tet-NH₂ were added at a 1:4:1 molar ratio at 0.5 mmol Tet per g of HATBA and reacted overnight at room temperature. The HATet solution was then dialyzed with SpectraPor dialysis tubing, frozen, and lyophilized. ¹H NMR spectroscopy analysis confirmed that ˜40% of HATet repeat units were functionalized with Tet. The percentage of modification was calculated by comparing the integral of the methyl HA peaks between δ 1.8 to 2.0 ppm to the aromatic proton peak of tetrazine between & 10.4 ppm (Koshy et al., Adv. Healthc. Mater. 5, 541-547. 2016).

### HANorDWIVA

Hydroxyl groups in HANor were first modified with methacrylates (Me) to form HANorMe via esterification with MA by adapting a previously described protocol (Smeds and Grinstaff, “Photocrosslinkable polysaccharides for in situ hydrogel formation” 2000). HANor was dissolved (1% w/v) in distilled water at 4° C. A 15-fold molar excess of MA was added while maintaining pH between 8.5 and 9.0. After all MA was added, the solution was left overnight at room temperature. The HANorMe solution was then dialyzed with SpectraPor dialysis tubing, frozen, and lyophilized. H NMR spectroscopy analysis confirmed that ˜60% of HANorMe repeat units were functionalized with Me. The percentage of modification was calculated by comparing the integral of the HA peaks between δ 1.8 to 2.0 ppm to the alkene proton peaks of methacrylates between δ 5.5 to 6.5 ppm (Yousefi et al., Appl. Spectrosc 2018).

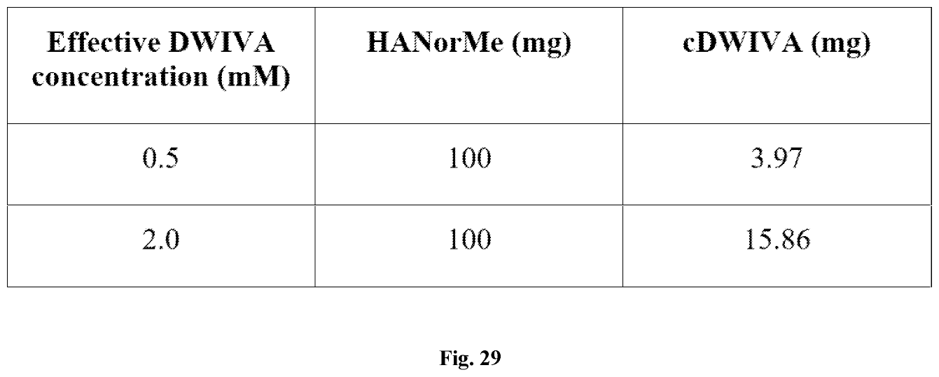

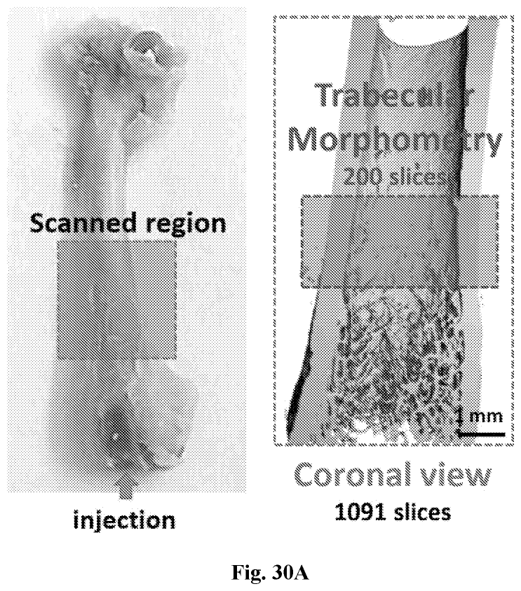

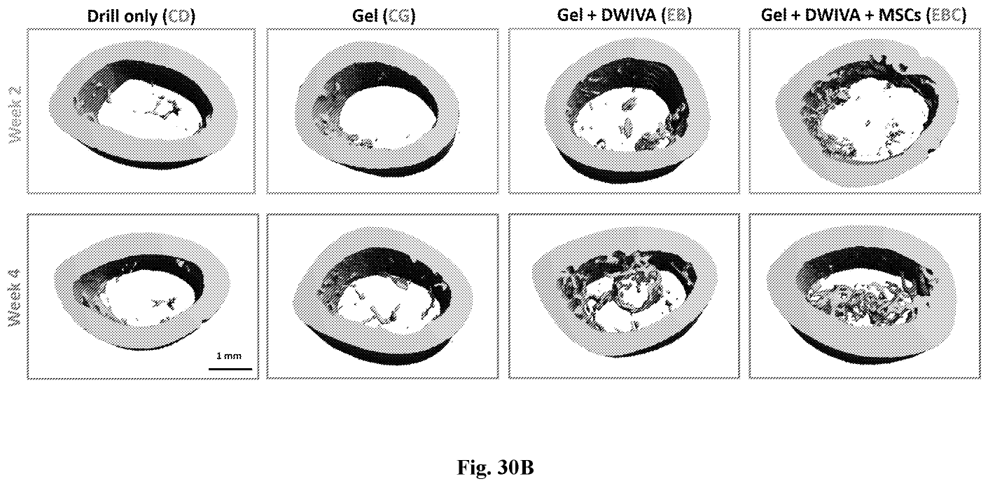

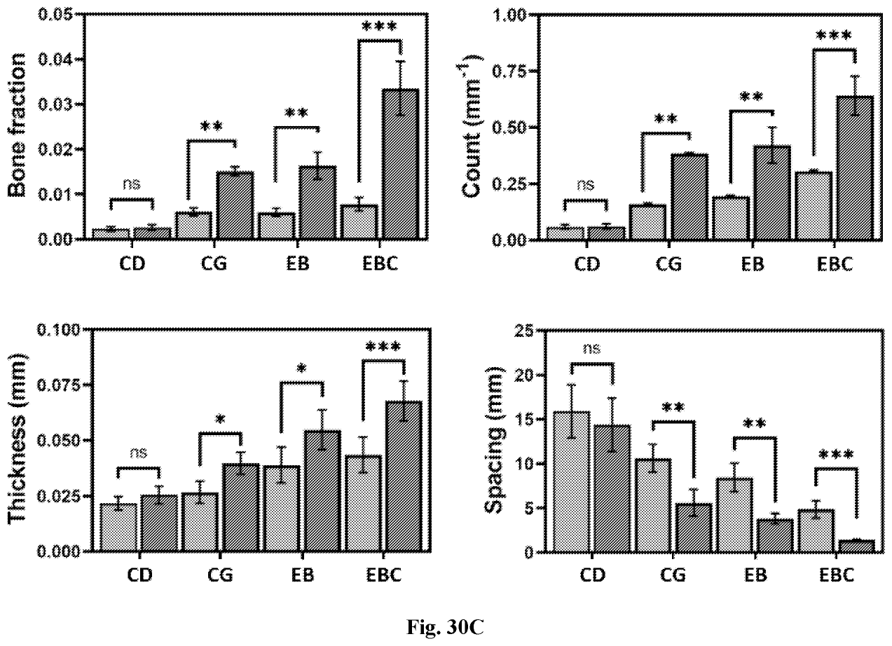

To peptide-functionalize HANorMe, thiols in cysteine-containing peptides were coupled to Me groups in HANorMe via an aqueous Michael addition reaction (Li et al., 2010). Briefly, HANorMe was dissolved (1% w/v) in 200 mM TEOA buffer (pH 8) at room temperature. Thiolated DWIVA sequence: GCGGGDWIVAG) in solution (50 mM in PBS) was added dropwise to reach a final DWIVA concentration that corresponds to an effective concentration of either 0.50 mM or 2 mM for 2 wt % HA Nor-Tet hydrogels (FIG. 29). The HANorDWIVA solution was then dialyzed with SpectraPor dialysis tubing, frozen, and lyophilized. 1H NMR spectroscopy analysis confirmed that 10% (for 0.5 mM) and 40% (for 2 mM) of Me groups were coupled with DWIVA. The percentage of Me-DWIVA coupling was calculated by comparing the integral of alkene proton peaks of methacrylate between δ 5.5 to 6.5 ppm (Yousefi et al., Appl. Spectrose, 2018) to the protons in the amino group of cysteine moiety between δ 0.50 to 1.50 ppm (Madl et al., Biomacromolecules 15, 445-455, 2014).

### Characterization of Nor-Tet Hydrogels

Rheological properties of Nor-Tet hydrogels were measured using a Discovery Hybrid Rheometer (DHR-3, TA Instruments, New Castle, DE) with a 20 mm diameter 1° cone upper plate geometry and a lower plate temperature of 37° C. Samples were prepared by dissolving HANor (with or without DWIVA functionalization) and HATet separately in PBS, followed by mixing, resulting in a final macromer concentration of 2 wt %. Immediately after mixing, 40 μl of the solution was pipetted to the center of the rheometer stage and the shear storage (G′) and loss (G″) moduli were monitored. To measure plateau G′ and gelation kinetics, time sweeps were performed for 1 hour at 1 Hz and 1% oscillatory strain. Bulk mechanical properties of self-forming hydrogels were measured using an EZ-SX Mechanical Tester (Shimadzu, Long Beach, CA) equipped with a 50 N compression load. Briefly, hydrated HANor or HANorDWIVA and HATet were mixed and pipetted into cylindrical polydimethylsiloxane (PDMS) molds (2 mm height, 8 mm diameter). The hydrogels were allowed to form for 30 minutes before incubating in PBS overnight at 37° C. Formed cylindrical Nor-Tet hydrogels were compressed until 30% strain, and the elastic modulus € was determined using the slope of the stress-strain curve between 10% and 20% strain.

### Cell Culture

Human primary MSCs were cultured in 100 mm petri dishes in Growth Medium (MEM-α supplemented with 10% FBS and 1% P/S). To study the effects of soluble DWIVA on stem cell cultures, MSCs were seeded on top of glass coverslips (12 mm diameter) at a density of 3,000 cells per cm². After 6 hours in culture, media was replaced with either Growth Medium (0 mM DWIVA), Growth Medium supplemented with low (0.50 mM) or high (2.0 mM) DWIVA, or OS Medium.

To study the effects of tethered DWIVA on 2D hydrogel cultures, 2 wt % HA Nor-Tet hydrogels with varying degrees of DWIVA functionalization (0, 0.5, 2 mM) were formed on PDMS molds (11 mm diameter, 1 mm height). MSCs were seeded on top of these hydrogels at a density of 3,000 cells per cm². To study the effects of DWIVA in 3D hydrogel cultures, MSC-laden (2×10⁶ cells/ml) 2 wt % Nor-Tet hydrogels with varying degrees of DWIVA functionalization (0, 0.5, 2 mM) were formed on PDMS molds (8 mm diameter, 2 mm height). Appropriate media were replenished every 48 hours and all experiments were cultured for 7 days.

### Staining and Imaging-Based Analysis

After 7 days in culture, samples were fixed with 10% neutral buffered formalin for 10 mins (glass, 2D hydrogels) or 30 mins (3D hydrogels) at room temperature. To visualize cellular alkaline phosphatase (ALP), fast blue was added for 1 hour (glass, 2D hydrogels) or 3 hours (3D hydrogels). To visualize individual nuclei, samples were stained for double stranded DNA with Hoechst (1:1000) for 5 mins (glass, 2D hydrogels) or 15 mins (3D hydrogels). Acquisition of immunofluorescence images was performed with a Nikon A1 confocal microscope. Images were taken at 20× magnification and at laser wavelengths of 405 nm (nuclei) and 640 nm (ALP) (glass, 2D and 3D hydrogels). Image stacks were taken at height of 200 μm with a step size of 3.2 μm (3D hydrogels).

Mean fluorescence intensity (MFI) of cellular ALP was determined using ImageJ software (National Institute of Health, Bethesda, MD, USA). Briefly, nuclei stacks were binarized using the Otsu thresholding method. Binarized nuclei stacks were dilated, and the nuclei stacks were subtracted from the dilated stacks resulting in rings. The rings were converted into masks and overlayed on the ALP channel stacks. The 3D Objects Counter feature was then applied to calculate the surface area of the rings and the Measure function was used to determine the integrated density of the rings. The MFI value of every cell was calculated by dividing its mean grey value by area. The MFI values of Growth Medium and OS Medium groups were processed using a k-means clustering algorithm to determine the boundary between MSCs with low (ALP-negative) and high (ALP-positive) MFI. The selected threshold MFI covers 95% of ALP-negative cells (Vega et al., J. Biomol. Screen. 17, 1151-1162. 2012).

### Animal Protocol

The animal experiment was approved by the Cooper University Health Care's Institutional Animal Care and Use Committee (IACUC). Experiments were performed on 8-week-old male Lewis rats (Charles River Laboratories, Raleigh, NC) weighing approximately 275-300 g. The rats were housed in a 12-hour dark-light cycle where they had access to food and water ad libitum. The rats were randomly divided into three groups: drilled canal alone, injectable hydrogel, and injectable hydrogel with 2 mM DWIVA peptide. The procedures were performed under anesthesia using inhaled isofluorane solution and buprenorphine SR. Once anesthetized, surgical sites were prepared by shaving the ventral aspect of the abdomen and hind legs and scrubbed with 2% chlorohexidine and 70% isopropyl alcohol. A standard median parapatellar approach to bilateral knees was performed. The distal femurs were exposed, and the femoral notch was identified. Utilizing a 1 mm sterile drill, the intramedullary canal was drilled and checked on fluoroscopy for placement. Three passes with the drill were performed to adequately remove native bone marrow and trabecular bone. The femurs were then filled with 0.2 ml of hydrogel solution via injection using a 25-gauge syringe. Bone wax was placed over the hole after injection. Closure of the arthrotomy and skin was performed with sutures. Following closure of the surgical sites, animals were given meloxicam SR (2 mg/ml) for pain alleviation and subsequently returned to their housing where they were monitored until they recovered from the surgical anesthesia. All rats were euthanized 4 weeks post-injection and the left and right femurs were harvested. The femurs were fixed with 10% neutral-buffered formalin for 24 hours, washed with distilled water, and stored in 70% ethanol at 4° C. until micro-CT imaging.

### Micro-CT

The distal shaft (midshaft down to the metaphysis) of extracted femurs were scanned using micro-CT (microCT 45, ScancoMedical AG, Brüttisellen, Switzerland) at 10.4 μm isotropic resolution, with 55 kVp energy, and 400 ms integration time. This region was chosen to evaluate the effects of injectable DWIVA hydrogels on trabecular bone formation. At the center of the distal shaft, a 200-slice-thick volume of interest (VOI) was identified, Gaussian filtered (sigma=1.2, support=2), and bone was identified by applying a global threshold (220 mg hydroxyapatite per cm³). Manufacturer-provided software for 3D standard microstructural analysis was used to generate 3D axial views of the 200-slice-thick VOIs and coronal views of the 1031-slice-thick distal shaft.

### Statistical Analysis

Statistical analysis was performed using GraphPad Prism (version 9.3.1, GraphPad Software, Inc., La Jolla, CA). All experiments were carried out in triplicate and single cell analysis was done with at least 50 cells per group. All graphs represent mean±standard deviation (SD). Analysis of variance (ANOVA) was performed followed by Tukey's test for post-hoc analysis. Differences among groups are stated as p<0.05 (*), p<0.01 (**), p<0.001 (***), and (ns) when differences between groups are not statistically significant.

### Example 2-4: Mechanics and Gelation Kinetics of Injectable HA Hydrogels are Preserved After DWIVA Coupling

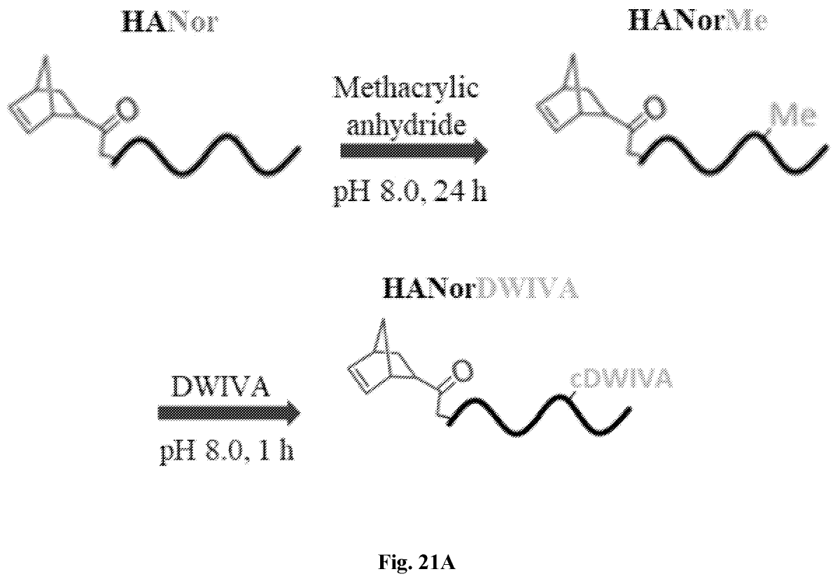

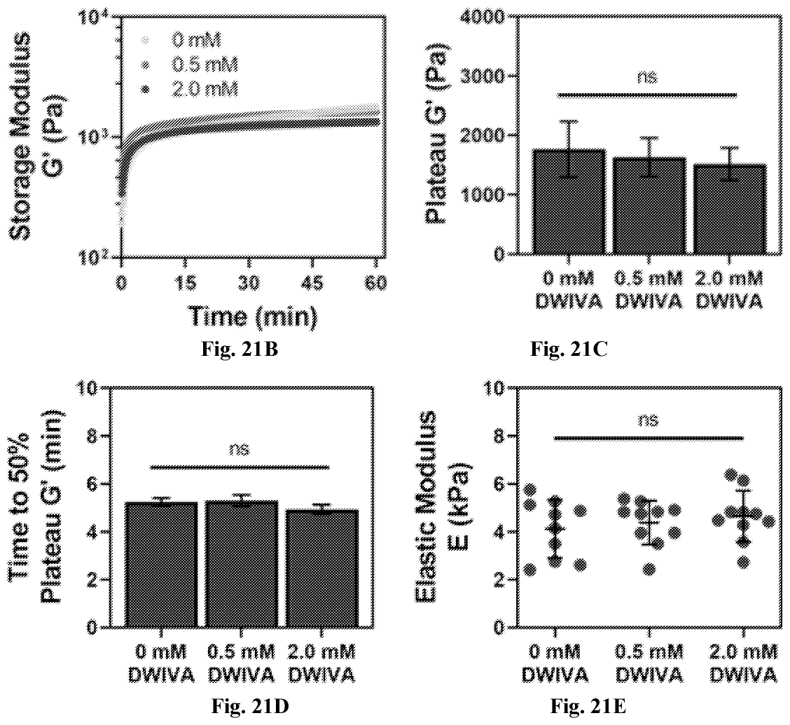

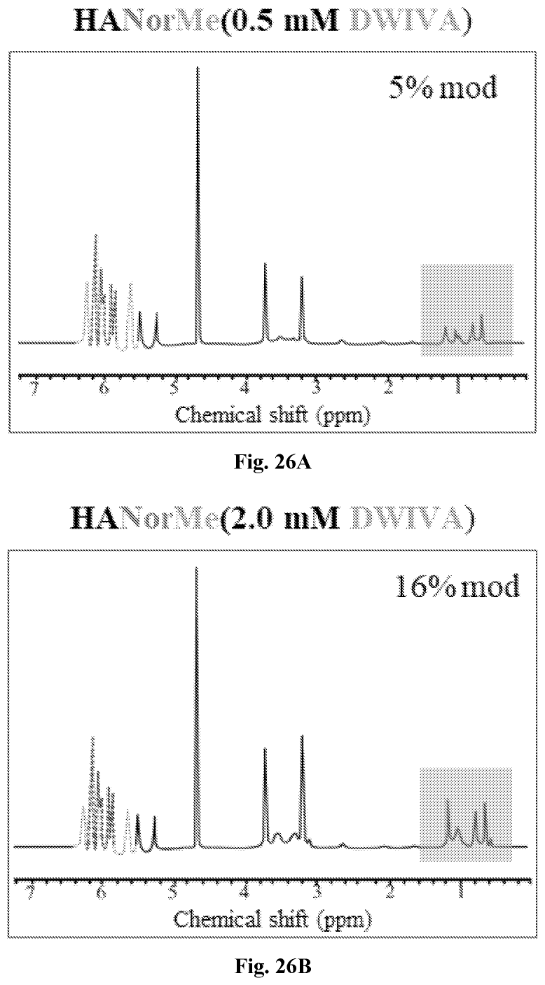

The percentage modification of HANor and HATet with Nor and Tet was determined to be ˜50% and ˜40% through 1H-NMR, respectively. Modification of HANor with methacrylates (Me) provide sites for coupling thiolated DWIVA peptides to the HANor macromer backbone (FIG. 21A). 1H-NMR shows that HANor has a Me modification of ˜80%. Coupling HANorMe with 0.5 and 2.0 mM DWIVA results in 5% and 16% of Me groups bound to peptide, respectively (FIGS. 26A-26B). Material characterization of Nor-Tet hydrogels functionalized with 0, 0.5, or 2.0 mM DWIVA shows that mechanics and gelation time are not affected by the DWIVA peptide. Frequency sweep rheology (0.1 to 10 Hz) at 37° C. shows constant storage modulus (G′) at every time point for Nor-Tet hydrogels with 0, 0.5, and 2.0 mM DWIVA, respectively (FIG. 21B). Time sweep rheology (1 Hz) at 37° C. that plateau G′ values are 1,767±468 Pa, 1,630=323 Pa, and 1,517±268 Pa for Nor-Tet hydrogels with 0, 0.5, and 2.0 mM DWIVA, respectively (FIG. 21C). The time to 50% plateau G′ values are 5.25±0.16 min, 5.30±0.24 min, and 4.93±0.20 min for Nor-Tet hydrogels with 0, 0.5, and 2.0 mM DWIVA, respectively (FIG. 21D). Compression testing shows elastic modu€ (E) values of 4.12±1.22 kPa, 4.38±0.92 kPa, and 4.64±1.07 kPa for Nor-Tet hydrogels with 0, 0.5, and 2.0 mM DWIVA, respectively (FIG. 21E).

### Example 2-5: Soluble Presentation of DWIVA Enhances ALP Levels of 2D MSC Cultures

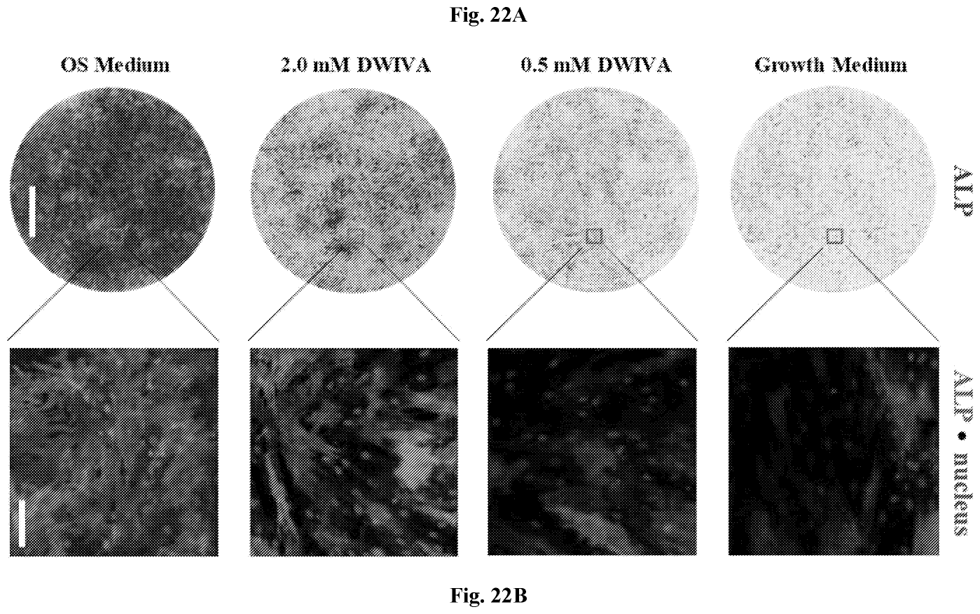

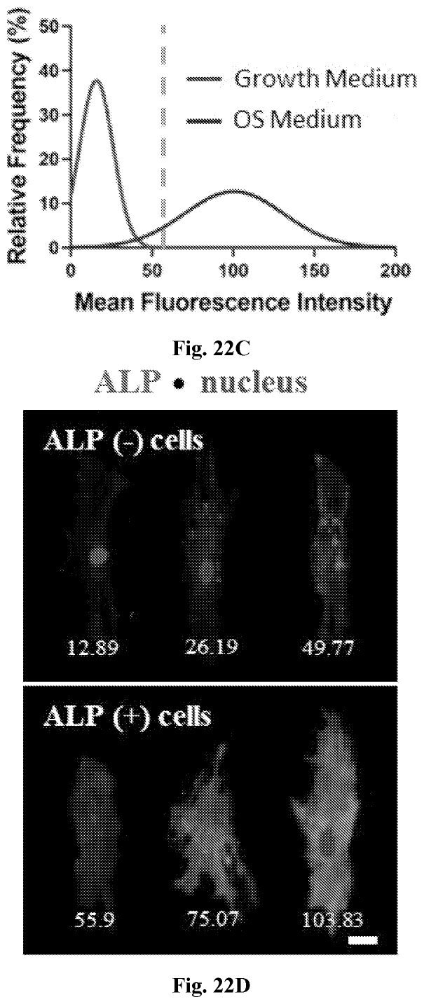

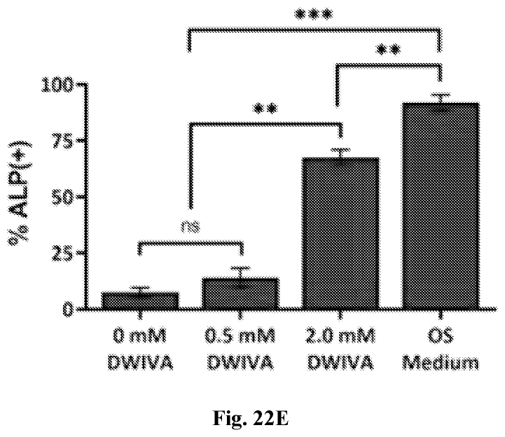

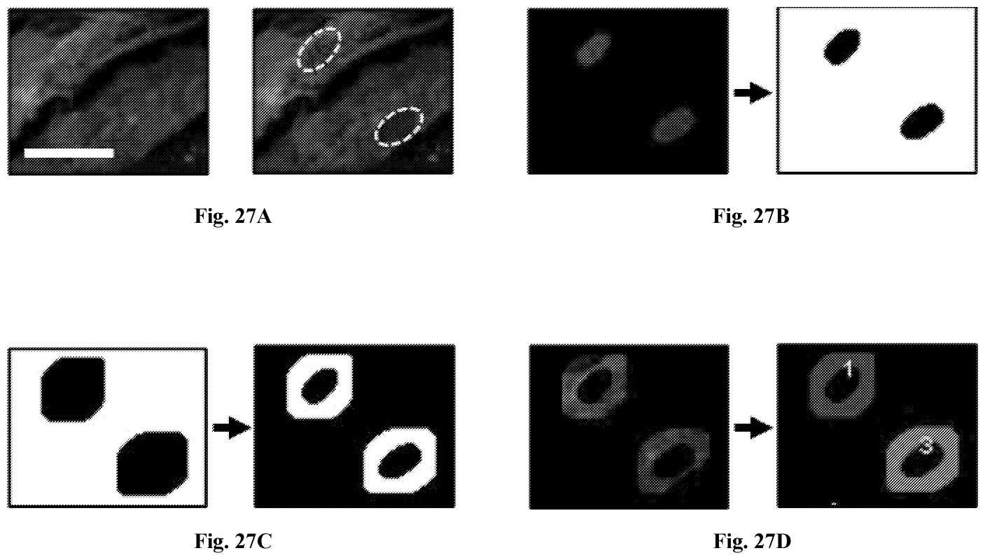

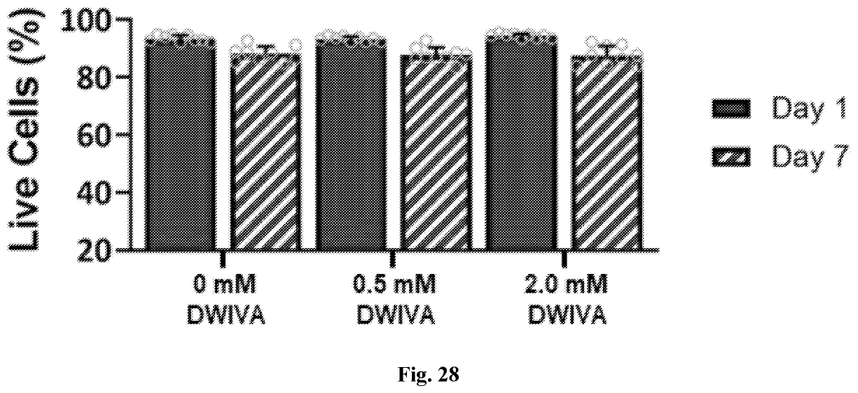

MSCs seeded on glass cultured in Growth Medium supplemented with 2.0 mM DWIVA show significantly more positive staining for ALP (dark blue) compared to cells cultured in Growth Medium or in Growth Medium supplemented with a lower (0.50 mM) DWIVA concentration (FIG. 22A). Confocal images of MSC cultures stained for ALP (magenta) and nuclei (blue) show a progressive increase in ALP signal with increasing DWIVA concentration (FIG. 22B). To distinguish between ALP(+) and ALP(−) MSCs, the mean fluorescence intensity (MFI) of intracellular ALP was first determined for individual MSCs using imaging-based analysis with ImageJ (FIGS. 27A-27D). The MFI values of the Growth Medium and OS Medium groups were processed using a k-means clustering algorithm to determine the boundary ALP(−) and ALP(+) clusters, resulting in an MFI cutoff value of 52, which covers 95% of ALP(−) MSCs (FIG. 22C). The MFI threshold value is used as a filter that assigns MSCs as ALP(−) (MFI<52) or ALP(+) (MFI≥52) (FIG. 22D). Using this technique, the percentage of ALP(+) MSCs on glass is 7.5±2.1% (Growth Medium), 14.1±4.3% (0.5 mM DWIVA), 67.5±3.7% (2.0 mM DWIVA), and 92.1±3.9% (OS Medium) (FIG. 22E).

### Examples 2-6: Immobilized DWIVA Enhances ALP Levels of MSCs on 2D Nor-Tet Hydrogels

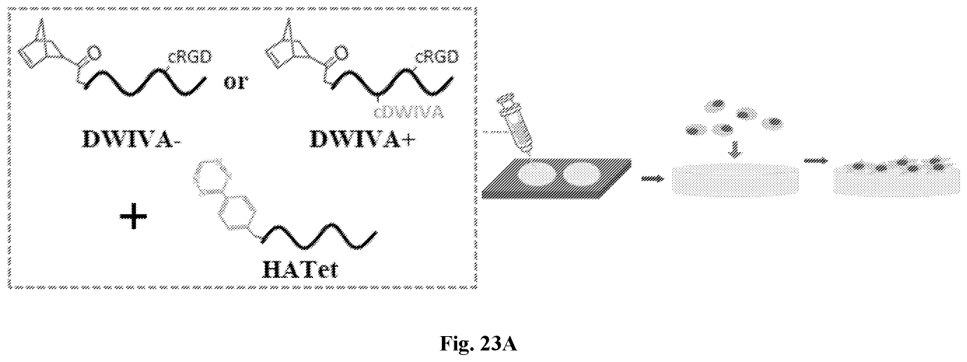

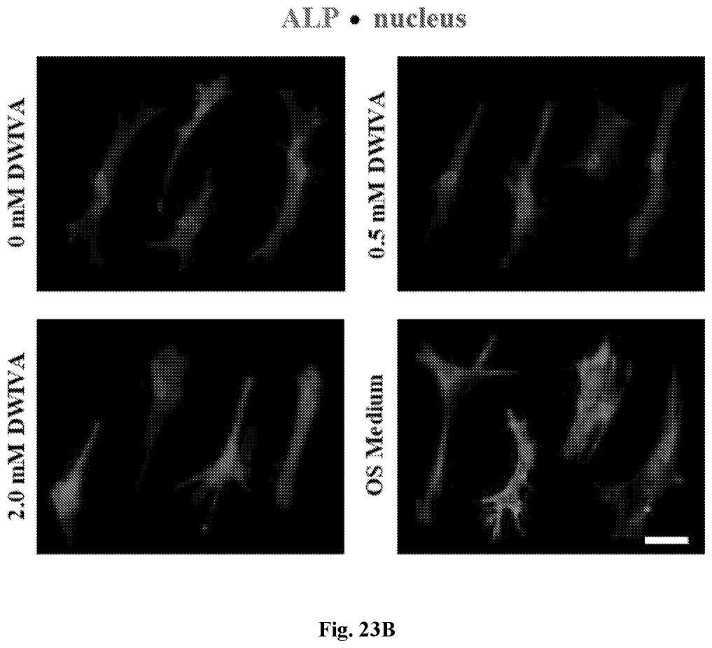

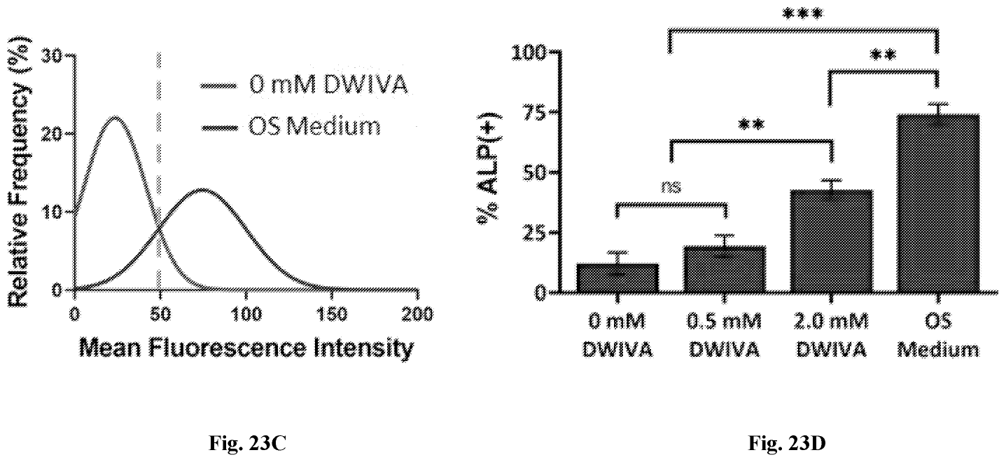

HANorMe was pre-functionalized with thiolated RGD (2 mM) and with 0, 0.5, or 2 mM thiolated DWIVA. Pre-functionalized HANorMe macromers were then mixed with HATet to form Nor-Tet hydrogels that were seeded with MSCs (FIG. 23A). Representative MSCs (ALP, magenta; nuclei, blue) in different treatment groups show more magenta signal in cells atop 2D hydrogels coupled with 2.0 mM DWIVA compared to cells atop hydrogels with lower DWIVA (0, 0.5 mM) concentrations (FIG. 23B). A k-means clustering algorithm was applied for MFI values of MSCs on Growth Medium (negative group) and OS Medium (positive group) to determine an MFI cutoff of 50, which covers 95% of ALP(−) MSCs (FIG. 23C). The percentage of ALP(+) MSCs on Nor-Tet hydrogels is 12.1±4.5% (0 mM DWIVA), 19.3±4.5% (0.5 mM DWIVA), 42.9±3.8% (2.0 mM DWIVA), and 74.1±4.1% (OS Medium) (FIG. 23D).

### Example 2-7: Immobilized DWIVA Enhances ALP Levels of MSCs in 3D Nor-Tet Hydrogels

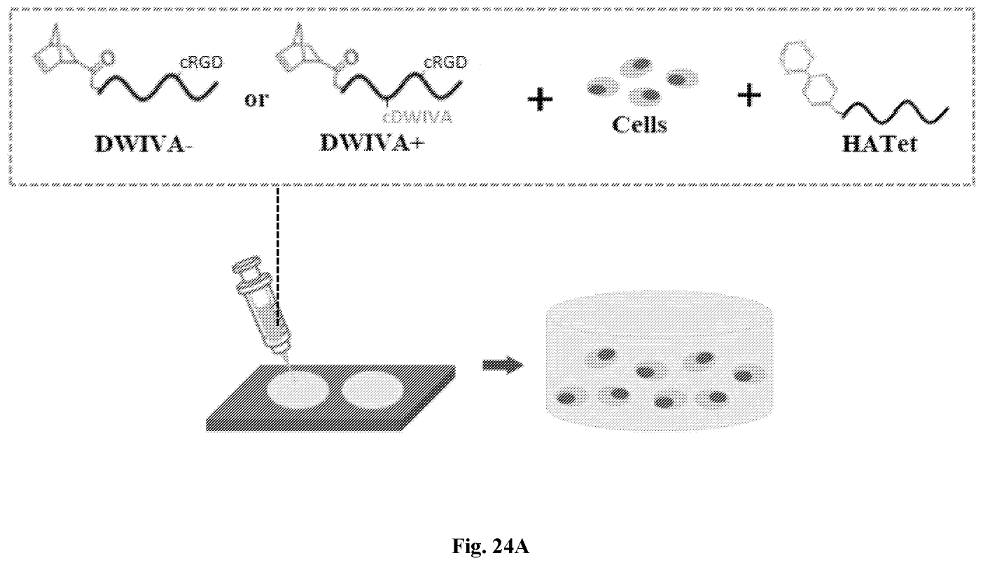

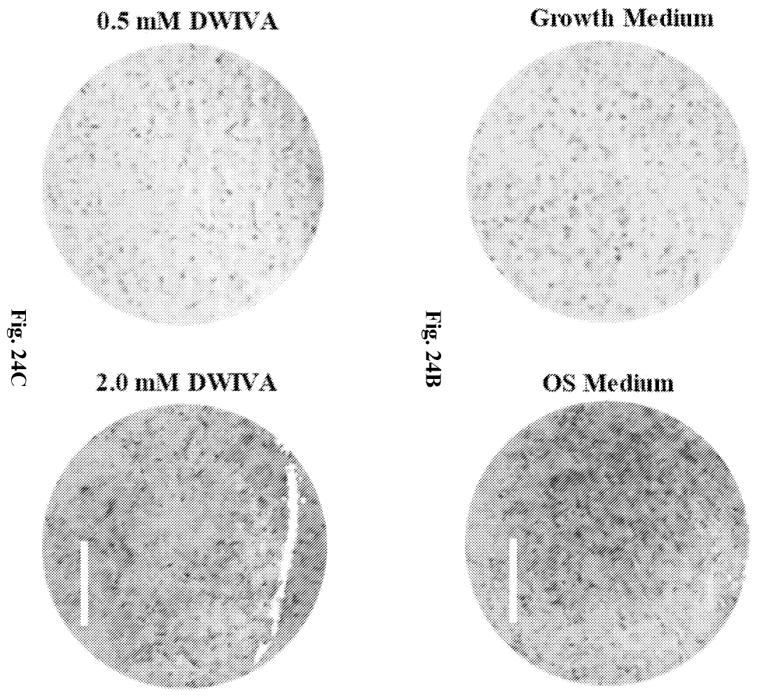

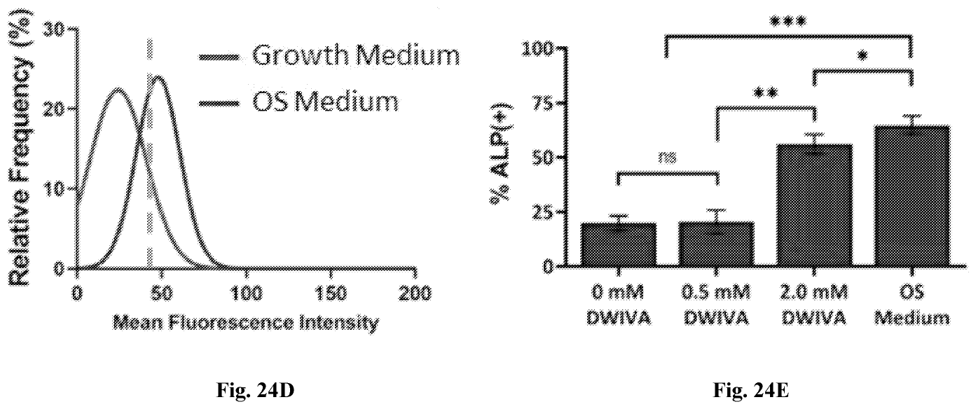

MSCs were mixed in HATet and HANor pre-functionalized with thiolated RGD (2 mM) and with 0, 0.5, or 2 mM thiolated DWIVA and added to cylindrical molds to form 3D cell-laden Nor-Tet hydrogels (FIG. 24A). MSCs encapsulated in 0 mM DWIVA hydrogels and cultured in Osteogenic Medium show significantly more positive staining for ALP (dark blue) than those cultured in Growth Medium (FIG. 24B). MSCs cultured in Growth Medium and encapsulated in DWIVA-functionalized HA Nor-Tet hydrogels show an increase in ALP signal with increasing DWIVA concentration (FIG. 24C). A k-means clustering algorithm was applied for MSCs in 3D hydrogels in Growth Medium (negative group) and OS Medium (positive group) to determine a threshold MFI of 48, which covers 95% of ALP(−) MSCs (FIG. 24D). The percentage of ALP(+) MSCs in 3D Nor-Tet hydrogels is 19.7±3.5% (Growth Medium), 20.4±5.5% (0.50 mM DWIVA), 56.1±4.6% (2.0 mM DWIVA), and 64.7±4.3% (OS Medium) (FIG. 24F). Confocal images of MSCs cultured in Growth Medium and encapsulated in Nor-Tet hydrogels without (0 mM) or with (2 mM) DWIVA tethering show observable differences in ALP fluorescence (FIG. 24F).

### Examples 2-8: Injectable HA Hydrogels with Immobilized BMP-2 Induce Trabecular Bone Growth

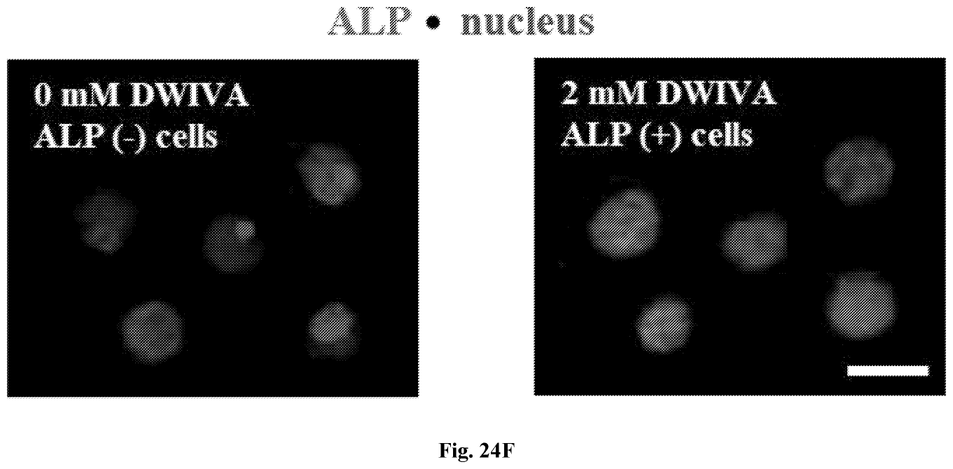

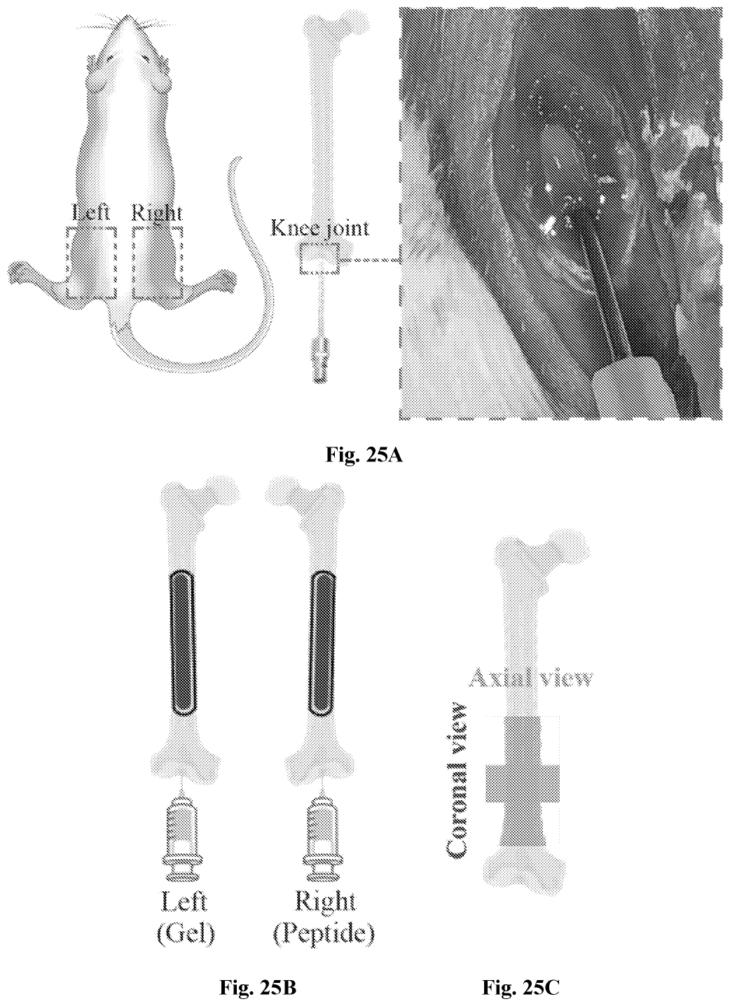

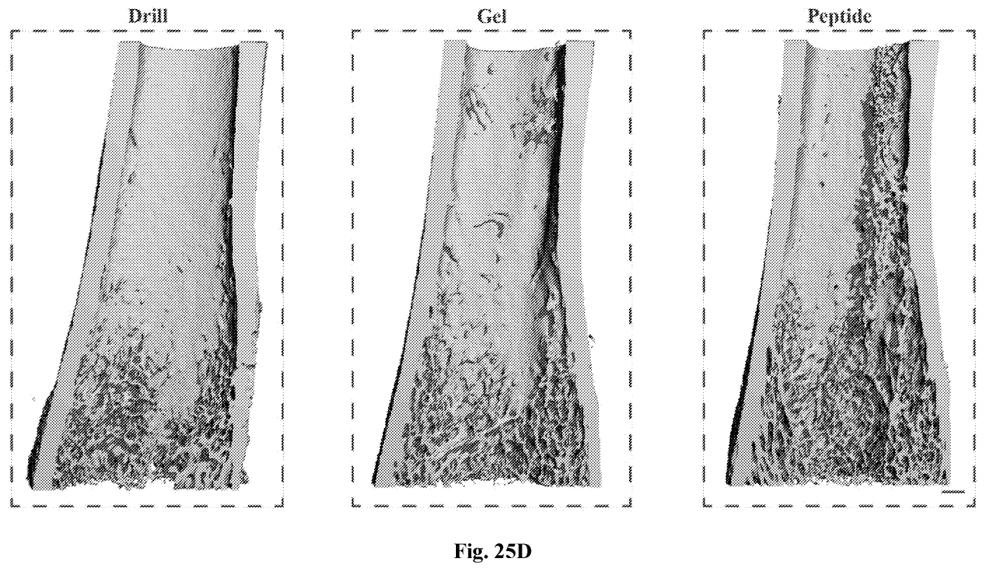

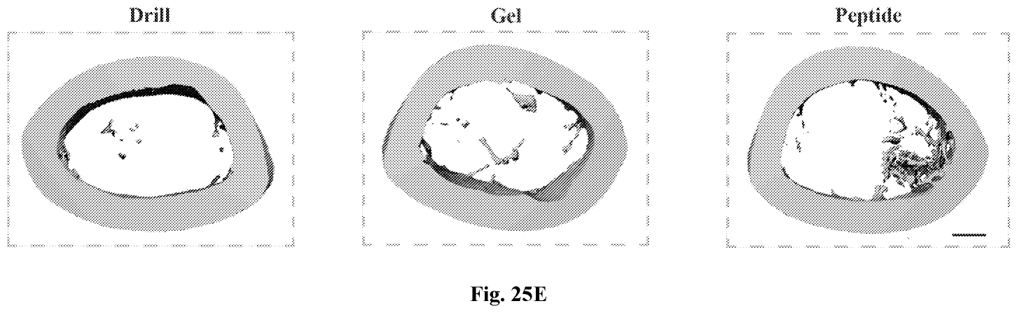

To evaluate bone growth in vivo, the left and right knee joints of 8-week-old male Lewis rats were opened surgically under sterile conditions. The intercondylar notch of each distal femur was identified, and the intramedullary space of the femur was cleared of native trabecular bone and bone marrow using a 1 mm drill. (FIG. 25A). Nor-Tet hydrogel solutions without peptide (Gel group) and with 2.0 mM DWIVA peptide (Peptide group) were injected into the left and right femurs, respectively (FIG. 25B). Four weeks post-injection, rats were euthanized, and femurs were harvested. Microcomputed tomography (micro-CT) was used to image the distal shaft and to create 3D coronal views of the distal shaft and 3D axial views of the distal shaft center (FIG. 25C). Increased trabecular bone growth is observed in femurs injected with Peptide-containing Nor-Tet hydrogels when compared to femurs injected with Gel alone, as seen by 3D coronal (FIG. 25D) and 3D axial (FIG. 25E) views.

### Example 2-9

In the present study, DWIVA-functionalized self-forming hydrogels that enhance osteogenesis in vitro and induce bone growth in vivo were developed. The hydrogel macromers consist of HA modified with Nor or Tet moieties which upon mixing form covalent networks by undergoing a Diels-Alder reaction. While this chemistry has been used to develop biocompatible poly(ethylene glycol), alginate, and gelatin self-forming hydrogels, it is challenging to incorporate bioactive motifs into Diels-Alder hydrogels. Here, the carboxyl groups of HA were modified with Nor and Tet, leaving the hydroxyl groups in HA free for additional modifications. This allowed us to pre-functionalize HANor with DWIVA by first modifying hydroxyl groups on HANor with Me moieties, followed by coupling with thiolated DWIVA via a Michael-addition reaction (FIG. 21A). Since DWIVA was coupled to hydroxyl groups, it was hypothesized that this should have no impact on Nor-Tet interactions from moieties coupled to carboxyl groups of HA. To test the hypothesis, the present study performed rheological tests of 2 wt % HA Nor-Tet hydrogels formed by mixing HATet with HANorMe pre-coupled with different concentrations of DWIVA (0, 0.5, 2.0 mM) (FIG. 21B-21D). While there was a slight decrease in the storage modulus (G′) and gelation time, this was not a significant decrease and may be due to slight variability from time taken to mix and add solutions to the rheometer stage between samples. HA Nor-Tet hydrogels were also pre-formed in cylindrical molds, stored in PBS at 37° C. overnight, and compression testing was performed to measure the effects of DWIVA coupling on elastic moduli (E) (FIG. 21E). The hydrogels retained their cylindrical geometry at physiological temperature and DWIVA had no effect on stiffness.

DWIVA is the active motif in the wrist epitope of bone morphogenetic protein 2 (BMP-2), and growth medium supplemented with BMP-2 causes an osteogenic response on cultured cells. To evaluate the osteogenic properties of DWIVA, MSCs were seeded on glass and cultured in Growth Medium supplemented with low (0.5 mM) or (2.0 mM) concentrations of soluble DWIVA. After 7 days in culture, MSCs were stained for alkaline phosphatase (ALP), a well-established biomarker of osteogenesis. MSCs cultured in Growth Medium and in OS Medium served as control groups and had the lowest and highest observable amount of ALP, respectively (FIGS. 22A-22B). To quantify of ALP(+) MSCs, a mean fluorescence intensity (MFI) histogram of cytoplasmic ALP from MSCs cultured in Growth Medium (negative control) and OS Medium (positive control) was used to identify a cutoff MFI value for ALP(+) cells (FIG. 22C). The threshold MFI was set such that 95% of MFI values for MSCs in the ALP(−) group (Growth Medium) were classified as ALP(−). This technique is an adaptation of a k-means clustering-based approach to assign cells to a subpopulation based on specific metrics (Vega et al., J. Biomol. Screen. 17, 1151-1162 2012), and it minimizes bias for identifying ALP(+) MSCs. Using this approach for MSCs on glass, the threshold MFI was determined to be 52. The Growth Medium and OS Medium ALP MFI histograms had very little overlap and had peak values of 18 and 102, respectively (FIG. 22C). Although glass is extremely stiff and rigid substrates favor osteogenic differentiation, synergy between high mechanics and soluble was only observed at high (2.0 mM) DWIVA concentrations. There was no significant difference in ALP(+) MSCs between Growth Medium and low (0.5 mM) DWIVA concentration, while 67% of MSCs were ALP(+) in the high DWIVA concentration group (FIG. 22E).

After demonstrating that soluble DWIVA induces increased ALP, the osteogenic effects of immobilized DWIVA were evaluated Here, HANorMe was first pre-coupled with 2 mM thiolated RGD and different concentrations of thiolated DWIVA (0, 0.5, 2.0 mM). To form peptide-functionalized HA Nor-Tet hydrogels, bioactive HANor was mixed with HATet and the solution polymerized on cylindrical molds, and MSCs were seeded on top (FIG. 23A). Since MSCs do not readily adhere to HA, all groups included 2.0 mM RGD, a peptide motif of adhesive fibronectin. MSCs attached on all groups, and after 7 days increased intracellular ALP levels were observed with increasing DWIVA concentration (FIG. 23B). To minimize bias while identifying ALP(+) MSCs, MFI histograms of MSCs on HA Nor-Tet hydrogels cultured in Growth Medium (negative group) and OS Medium (positive group) were used to identify a threshold MFI (FIG. 23C). Through k-means clustering, the cutoff MFI for ALP(+) MSCs was determined to be 50, which is slightly lower than the threshold for the glass group (MFI≥52). There was also significantly more overlap between MFI values for the negative and positive groups, and the MFI peak for the OS Medium group decreased from 102 to 76 (FIG. 23C) The decrease in MFI for the positive group could be attributed to the mechanical properties of the HA Nor-Tet hydrogels. At a total macromer concentration of 2 wt %, HA Nor-Tet hydrogels were about 5 kPa, and this low stiffness does not osteogenesis. Despite being on soft hydrogels, almost 50% of MSCs on Nor-Tet hydrogels with a high concentration (2.0 mM) of immobilized DWIVA were ALP(+) (FIG. 23D). Future studies that evaluate the synergistic effects of higher mechanics and immobilized DWIVA on osteogenic differentiation may show increased ALP(+) cells at lower DWIVA concentrations.

To investigate the osteogenic effects of immobilized DWIVA in 3D environments, MSCs were suspended in pre-hydrogel solution consisting of HATet and HANor pre-functionalized with thiolated RGD and different concentrations of thiolated DWIVA (0, 0.5, 2.0 mM) and extruded into molds (FIG. 24A). Gross images of 0 mM DWIVA hydrogels stained for ALP show low and high levels of ALP expression of samples cultured in Growth Medium and Osteogenic Medium, respectively (FIG. 24B). These control groups confirm that encapsulated MSCs can express ALP in the presence of osteogenic growth factors. A qualitative observation between gross images of hydrogels cultured in Growth Medium with low (0.5 mM) and high (2.0 mM) DWIVA coupling display increased ALP staining with increasing DWIVA concentration (FIG. 24C). MSCs are not only highly viable in HA Nor-Tet hydrogels post-extrusion (FIG. 28) but respond to immobilized DWIVA in 3D self-forming hydrogels. Using the same k-means clustering approach for the 2D studies, the threshold MFI was determined to be 48, which is slightly lower than the glass (MFI≥52) and 2D hydrogel (MFI≥50) groups. Significant overlap between the negative (Growth Medium) and positive (OS Medium) relative frequency curves was also observed (FIG. 24D). Although percentage of ALP(+) MSCs increased with increasing DWIVA concentration, only about 50% of were classified as ALP(+), even for the OS Medium group (FIG. 24E). MSCs encapsulated in the HA Nor-Tet hydrogels remain spherical (FIG. 24F), while degradation-mediated cellular traction is necessary for cellular spreading and osteogenesis in 3D hydrogels. MSCs encapsulated in DWIVA hydrogels amenable to cell remodeling may be more osteoinductive than those in restrictive hydrogels like the HA Nor-Tet hydrogels used in this study. For example, MSCs in 3D gelatin-based Nor-Tet hydrogels are able to remodel the surrounding hydrogel, and replacing the HA backbone of HANorDWIVA with gelatin would allow for 3D cell spreading and DWIVA signaling.

After validating the osteogenic properties of DWIVA-functionalized HA Nor-Tet hydrogels in vitro, the present study evaluated the ability to grow bone tissue in regions prone to osteoporotic fragility fractures. Specifically, femur fractures can lead to a total hip replacement, and forming nascent trabecular bone in the femur would help prevent fractures from occurring in the first place. To examine the effectiveness of HA Nor-Tet hydrogels in locally regenerating bone in vivo, DWIVA-coupled self-forming hydrogels (2 mM) were injected into hollow rat femurs (FIGS. 25A-25B). A high DWIVA concentration was chosen since the percentage of ALP(+) MSCs was highest at 2.0 mM DWIVA. MSCs in HA Nor-Tet hydrogels without DWIVA functionalization expressed minimal ALP levels and this formulation was injected in contralateral femurs and served as a negative control group. 4 weeks post-injection, the femurs were harvested and microCT imaging was utilized to examine the differences in new trabecular bone growth in femurs that were kept hollow (Drill) or injected with uncoupled (Gel) or DWIVA-coupled (Peptide) self-forming hydrogels. 3D coronal views of the distal shaft for the Drill and Gel groups shows minimal trabecular bone along the shaft with trabeculae in the metaphysis (bottom), whereas in the Peptide group significant trabecular bone growth is observed along the shaft (FIG. 25D). 3D axial views of the distal shaft center reveal almost no trabecular bone in the Drill group, with increasing trabeculae seen in the Gel and Peptide groups (FIG. 25E). Since the Gel and Peptide groups were acellular, bone regeneration in the trabecular space would have had to be produced by native cells that migrated into the hydrogel. The migration of native cells into the trabecular space may have been aided by the presence of hyaluronidase, an enzyme that breaks down hyaluronic acid, in the femoral shaft. Enzymatic degradation of HA hydrogels in vivo could be replicated in vitro by adding exogeneous hyaluronidase. Together, these findings show that injectable, DWIVA-functionalized hydrogels can locally regenerate bone in vivo and present opportunities for follow-up studies. Bone regeneration after a fracture can take 6-12 weeks, and the osteogenic effects of DWIVA at later time-points has not been determined. The HA Nor-Tet hydrogels are amenable to 2D and 3D MSC culture, and the inclusion of DWIVA has an osteogenic effect on resident MSCs. Thus, it would be interesting to evaluate the synergistic effects of MSCs and DWIVA in regenerating bone in vivo. Besides the DWIVA wrist epitope of BMP-2, BMP-2 also has a knuckle epitope with the active sequence KIPKASSVPTELSAISTLYLG (KIPKA). The KIPKA sequence is believed to bind to BMP receptor II. Since both receptors I and II are involved in the cascade for BMP-2, it would be beneficial to explore the independent effect of KIPKA and the synergistic effects of KIPKA and DWIVA sequences in locally regenerating bone.

### Example 2-10

The present study developed an injectable hydrogel with DWIVA, an immobilized mimetic peptide of BMP-2, and demonstrated that it that can regenerate trabecular bone in femurs. It was first showed that the effective concentration of DWIVA peptide in self-forming hydrogels can be controlled by changing the of peptide added during coupling with HANorMe macromers. It was also confirmed that the bioactivity of the DWIVA peptide is preserved post-coupling by showing that ALP expression is enhanced in human MSCs seeded atop (2D) or within (3D) DWIVA-functionalized HA Nor-Tet hydrogels. This hydrogel system has the potential to be used as a targeted therapeutic to improve bone density locally, which is imperative in reducing the incidence of osteoporotic fragility fractures.

### Example 3-1: Mesenchymal Stem Cells Enhance Targeted Bone Growth from Injectable Hydrogels with BMP-2 Peptides

Osteoporosis is the most common chronic metabolic bone disease, and the prevalence of osteoporotic fractures is rapidly increasing with the aging population. While bisphosphonates can reduce bone loss and risk of fracture, these drugs are systemic, rely on long-term use, and patient compliance is low. Recombinant human bone morphogenetic protein-2 (BMP-2) is an FDA-approved protein that can offer a more targeted therapeutic than systemic treatments. DWIVA is a peptide sequence corresponding to the wrist epitope of BMP-2, and DWIVA-functionalized hydrogels feature osteoinductive properties in vitro and in vivo. This study reports that self-forming DWIVA-functionalized hydrogels injected into the intramedullary canal of rat femurs induce a local increase in trabecular bone in as little as two weeks. Increases in bone volume, trabecular thickness, and trabeculae count from DWIVA-laden hydrogels persist for at least four weeks, and the inclusion of mesenchymal stem cells (MSCs) significantly enhances the development of mineralized bone. Histological analysis of decalcified femurs also shows that hydrogel injections containing DWIVA peptide and MSCs stimulate unmineralized bone tissue formation and induce an increased count of osteoblasts and osteoclasts at the injection site after four weeks. Overall, the MSC-laden DWIVA peptide-functionalized hydrogels presented rapidly induce targeted bone formation and have the potential to form nascent bone within bones in jeopardy of an osteoporotic fracture such as the femur.

### Example 3-2

Osteoporosis is characterized by a progressive loss of bone mass from a misbalance between resorption and formation, resulting in an increased risk for bone fractures. 1 in 2 women and 1 in 5 men above the age of 50 will suffer from an osteoporotic fracture, and osteoporotic fractures occur every three seconds. In the United States, the number of osteoporotic fracture hospitalizations exceeds those for heart attacks and stroke, and over 50% of these injuries are hip fractures. A hip fracture occurs when the upper region of the affected femur breaks, resulting in disability, loss of independence, and a 25% chance of death within a year post-injury. Dual-energy x-ray absorptiometry (DEXA) is a commonly used, noninvasive test to measure bone mineral density and diagnose osteoporosis. Prophylactic treatments to prevent fracture include exercise, hip protectors, and adequate calcium and vitamin D intake. For higher risk patients, drugs that inhibit bone resorption (e.g., bisphosphonates, estrogen) or hormone therapy that increases bone formation (i.e., Teriparatide) are prescribed. Major limitations of these preventative treatments is that they are non-targeted (systemic), rely on long-term use, and patient compliance is low. Targeted interventions that rapidly promote increased bone mineral density in high-risk fracture sites such as the femoral head/neck would address these limitations but unfortunately do not exist.

Bone homeostasis relies on a dynamic equilibrium between bone formation and resorption, which is maintained by osteoclasts, osteoblasts, and osteocytes regulated via a myriad of physical and biochemical signals. Over 50 years ago it was first reported that unmineralized bone extracellular matrix (ECM) contains factors that stimulate bone formation, and since then these factors have been classified as bone morphogenetic proteins (BMPs). While at least 20 different human BMPs have been identified and isolated, BMP-2 and BMP-7 are the two most studied for bone formation, and the only FDA-approved BMP-based product is the INFUSE® Bone Graft by Medtronic. This bone grafting procedure has been on the market since 2002, and it consists of lyophilized recombinant human BMP-2 (rhBMP-2) that is reconstituted and added to an absorbable collagen sponge for treating tibial non-unions and assisting in spinal fusion surgeries. Despite its long clinical history, retrospective studies comparing vertebrae fusion rates with and without rhBMP-2 are inconclusive, and its side effect profile includes postoperative inflammation, ectopic bone formation, and hyperactive osteoclast-mediated bone resorption. These unwanted effects could be due to rhBMP-2 leakage outside of the implant site, off-target signaling, and supraphysiological rhBMP-2 dosing.

Hydrogels are a class of soft biomaterials that can be customized with specific properties for applications in tissue engineering and regenerative medicine. For instance, hydrogels can be synthesized with bioactive peptides that increase cell adhesion and promote stem cell differentiation. Peptide-functionalized hydrogels also feature a high degree of control over peptide concentration and location, and hydrogels formed with rhBMP-2 mimetic peptides can maximize the therapeutic properties of rhBMP-2 while minimizing off-target effects. The rhBMP-2 protein has two osteogenic receptor-binding regions that bind to BMP receptor I (BMPR-I) and BMP receptor II (BMPR-II), known as the wrist and knuckle epitopes, respectively. The DWIVA peptide sequence derived from the knuckle epitope of rhBMP-2 also has high binding affinity towards BMPR-I, and several studies have demonstrated that DWIVA-modified hydrogels induce osteogenic differentiation in vitro and in vivo.

Injectable hydrogels based on a Diels Alder reaction between norbornene and tetrazine modified hyaluronic acid macromers was developed in the study described in Example 1. These hydrogels can be used to inject cells through clinically used syringe needles without impacting viability, and peptides can be covalently bound to the hydrogel backbone without altering mechanical properties. Using this platform, the present study synthesized DWIVA-functionalized injectable hydrogels and found that encapsulated human mesenchymal stem cells (MSCs) exhibited increased alkaline phosphatase (ALP) expression in vitro, and acellular injections induced local mineralized bone formation in vivo after four weeks. Building from these findings, this study hypothesizes that the inclusion of MSCs within injectable DWIVA hydrogels will result in a more rapid and robust formation of bone ECM and will enhance bone homeostasis. To test this hypothesis, the present study injected different hydrogel formulations into the intramedullary canal of rat femurs and evaluated the effects of DWIVA and MSCs on nascent bone formation and homeostasis via bone morphometry and histomorphometry of explanted femurs two- and four-weeks post-injection.

### Example 3-3: Methods

### Injectable DWIVA-Hydrogel Synthesis

### Preparation of Injectable Hydrogel Groups

To form injectable hydrogels, norbornene-modified hyaluronic acid (HANor) or HANor functionalized with DWIVA (HANor-DWIVA) was mixed with tetrazine-modified hyaluronic acid (HATet) at a 2 wt % concentration to form hydrogels without (G group) and with (GP group) DWIVA peptides. For cellular groups, human MSCs (Lonza, passage 3) were suspended in HANor-DWIVA solution (2 million cells/ml) and mixed with HATet to form MSC-laden DWIVA peptide hydrogels (GPC group).

### HANor-DWIVA Synthesis

To synthesize HANor-DWIVA, sodium hyaluronate (NaHA, Lifecore, 60 kDa) was first converted to its tetrabutylammonium salt (HA-TBA) as described in Gramlich et al. (Biomaterials. 2013 December; 34 (38): 9803-11). HA-TBA was then used to synthesize HANor-DWIVA by HANor methacrylation (HANor-Me) followed by coupling thiolated DWIVA onto methacrylate groups in HANor-Me (HANor-DWIVA) (Gultian et al., Front Biomater Sci. 2022; 1:948493). To synthesize HANor, the carboxyl groups in HA-TBA were modified with norbornene via an amidation reaction with 5-norbornene-2-methylamine (0.4 mmol per gram of HA-TBA) in anhydrous DMSO (dimethyl sulfoxide, 2% w/v) and BOP (benzotriazole-1-yl-oxy-tris-(dimethylamino)-phosphonium hexafluorophosphate) under nitrogen for two hours at room temperature. To stop the reaction cold distilled water was added, and the solution was dialyzed (SpectraPor, 6-8 kDa molecular weight cutoff), frozen, and lyophilized, resulting in freeze dried HANor.

To synthesize HANor-Me, HANor was dissolved (1% w/v) in distilled water at 4° C., and hydroxyl groups in HANor were modified with methacrylates via an esterification reaction with methacrylic anhydride (MA) added in 15-fold molar excess dropwise while maintaining pH between 8.5 and 9.0 using 5 N sodium hydroxide. After all the MA was added, the solution was left stirring overnight at room temperature, dialyzed (SpectraPor, 6-8 kDa molecular weight cutoff), frozen, and lyophilized, resulting in freeze dried HANor-Me.

To synthesize HANor-DWIVA, thiols in thiolated DWIVA were reacted with methacrylate groups in HANor-Me via an aqueous Michael addition reaction. Briefly, HANor-Me was dissolved (1% w/v) in 200 mM TEOA (triethanolamine) buffer (pH 8) at room temperature, and thiolated DWIVA (sequence: GCGGGDWIVAG) in solution (50 mM in phosphate buffered saline, PBS) was added dropwise to reach a final DWIVA concentration that corresponds to an effective concentration of 2 mM for 2% w/v DWIVA-hydrogels (15.86 mg DWIVA per 100 mg HANorMe). (Madl et al., Biomacromolecules 15 (2): 445-455; Zhang et al., Bioorg. Chem. 116:105382). The solution was then dialyzed (SpectraPor, 6-8 kDa molecular weight cutoff), frozen, and lyophilized, resulting in freeze dried HANor-DWIVA.

### HATet Synthesis

To synthesize HATet, carboxyl groups in HA-TBA were modified with tetrazine by dissolving HA-TBA (1% w/v) in 100 mM MES (2-(N-morpholino) ethanesulfonic acid) buffer (pH 6), and EDC (1-ethyl-3-(3-dimethylaminopropyl) carbodiimide hydrochloride), NHS (N-hydroxysuccinimide ester), and tetrazine-amine were added at a 1:4:1 molar ratio at 0.5 mmol tetrazine-amine per gram of HA-TBA and reacted overnight at room temperature. The solution was then dialyzed (SpectraPor, 6-8 kDa molecular weight cutoff), frozen, and lyophilized, resulting in freeze dried HATet.

### Femoral Intramedullary Canal Injection Model

To study the effects of DWIVA peptides and the combined effects of DWIVA peptides and MSCs on nascent bone formation, hydrogel injections into rat femurs were used due to the prevalence of femur fractures and similarities in bone anatomy between rats and humans (Schindeler et al., J. Orthop. Res. 36 (3): 832-840). Prior to starting, the animal studies were reviewed and approved by the Cooper University Health Care's Institutional Animal Care and Use Committee (IACUC). 275 to 300 gram 8-week-old male Lewis rats (Charles River Laboratories) were randomly placed in hydrogel (G), hydrogel with peptide (GP), or hydrogel with peptide and MSCs (GPC) cohorts. To prep for hydrogel injections, rats were anesthetized, and the intramedullary canal of test femurs were cleared of bone marrow and trabecular bone by drilling (1 mm drill bit) through the intercondylar notch using fluoroscopy for guidance. For each cohort, 300 μl of norbornene-containing and tetrazine-containing hydrogel solutions (2 wt %) were loaded onto separate syringes. Test femurs were filled with 200 μl of hydrogel solution which was prepared by mixing norbornene and tetrazine macromer solutions using a Luer Lock coupler and injecting into the intercondylar notch with a 25-gauge syringe needle. Post-injection, the drilled opening was covered with bone wax, and closure of the arthrotomy and skin was completed using Vicryl sutures After two or four weeks, rats were euthanized, the test and contralateral femurs were extracted, fixed with 10% neutral-buffered formalin for 24 hours, washed with distilled water, and stored in 70% ethanol at 4° C.

### Bone Morphometry Analysis

Cortical and trabecular mineralized bone in extracted femurs was analyzed using micro computed tomography (microCT 45, ScancoMedical AG, Brüttisellen, Switzerland) at 10.4 μm isotropic resolution, with 55 kVp energy, and 400 ms integration time.

### Example 3-4: Hydrogel Injections Remain in the Intramedullary Canal of Rat Femurs

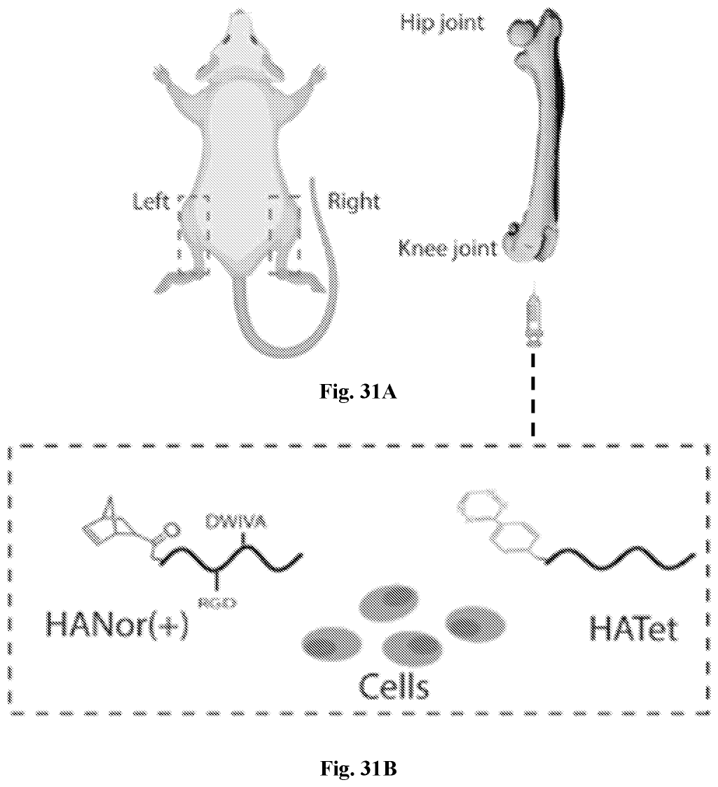

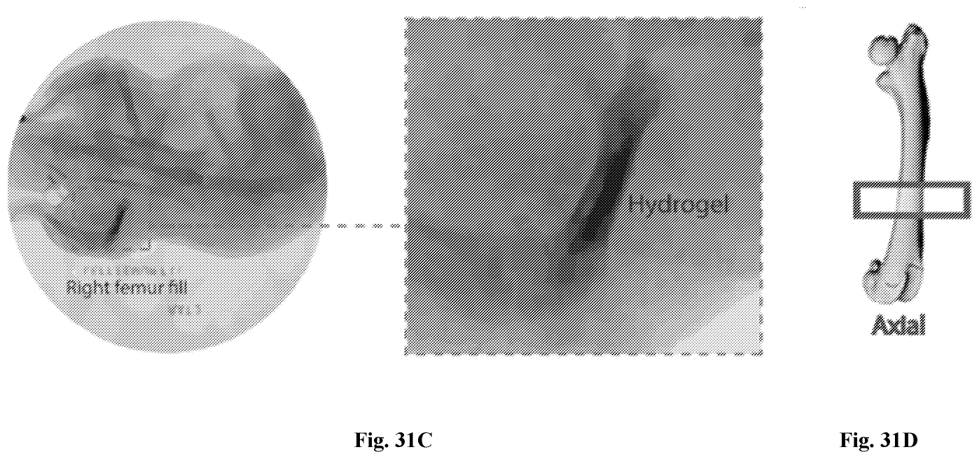

To assess targeted bone formation in rat femurs, the stifle joints of 8-week-old male Lewis rats were surgically opened under sterile conditions. After proper identification of the intercondylar notch, the intramedullary space was cleared of native trabecular bone and bone marrow and then filled with one of three therapeutic hydrogel solutions: hydrogel alone (G group), hydrogel with 2.0 mM DWIVA peptide (P group), and hydrogel with 2.0 mM DWIVA peptide co-delivered with MSCs (PC group). The left femur was used as a control, and the right femur was injected with a hydrogel solution (FIGS. 31A-31B). Femurs were harvested at two- and four-week post-injection and immediately scanned using micro-CT (FIG. 31C). Images show that the hydrogel filled the previously emptied intramedullary space of the distal shaft. Additionally, morphometry and histomorphometry analyses of trabecular and cortical bone growth, as well as the quantification of osteoblast and osteoclast formation were evaluated as functions of time and hydrogel composition with and without encapsulated MSCs.

### Example 3-5: Injectable DWIVA Hydrogels Induce Local Mineralized Trabecular Bone Formation

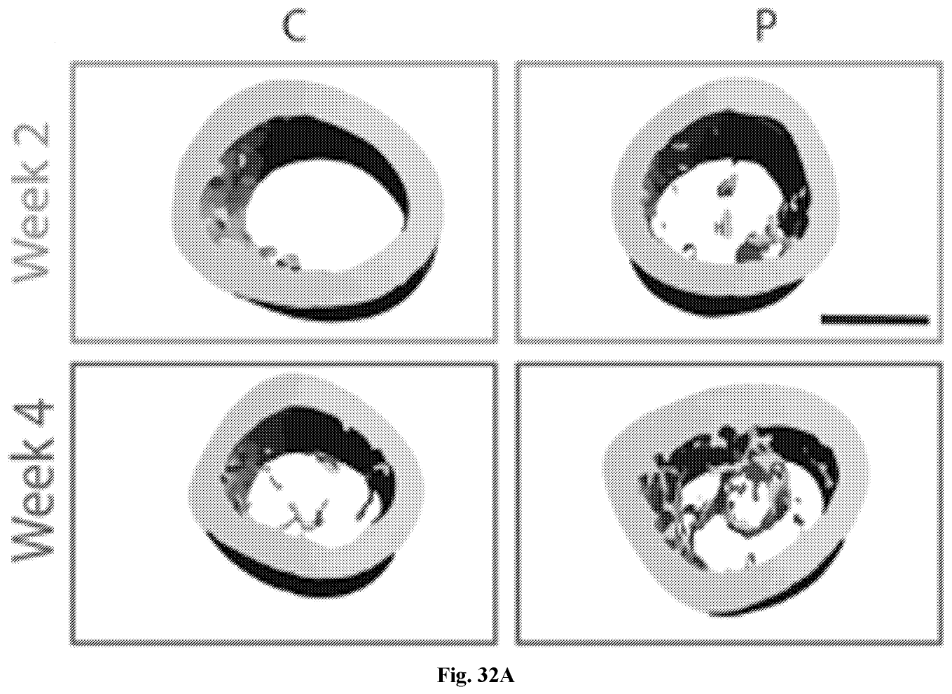

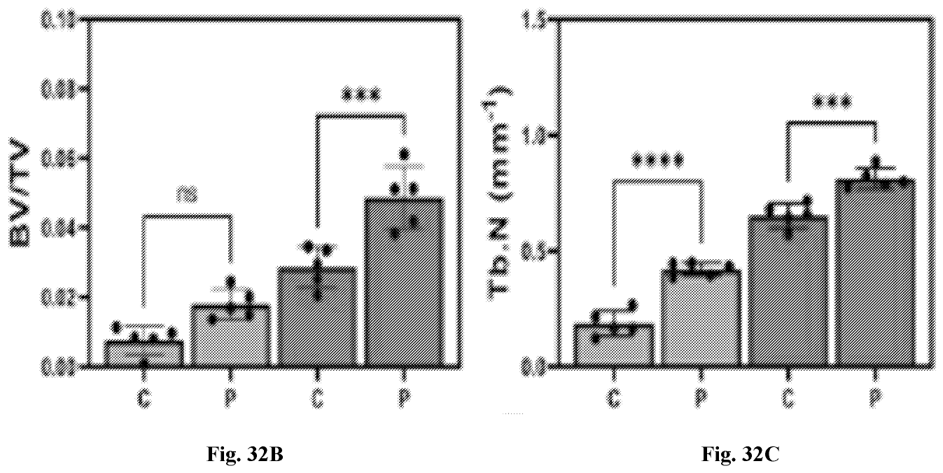

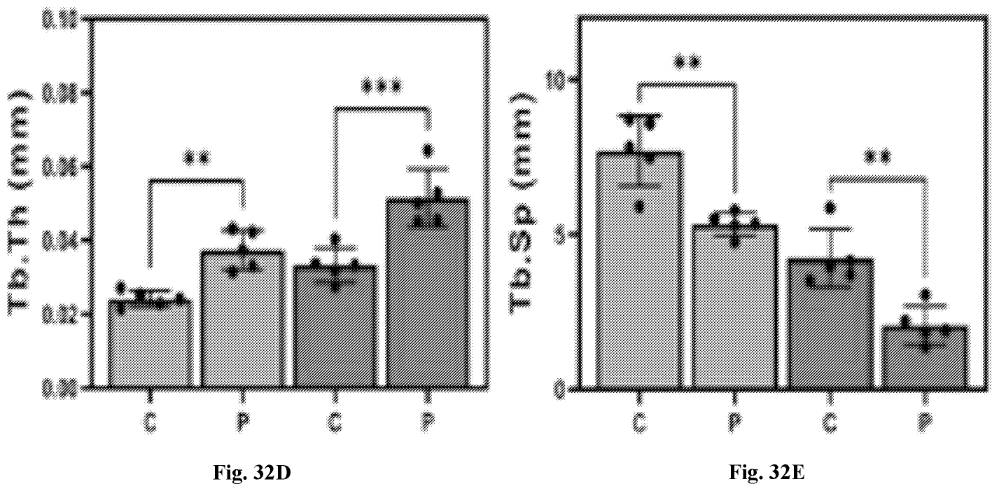

Analysis of trabeculae morphometry revealed increased bone growth in hydrogels functionalized with DWIVA peptide relative to hydrogels without peptides. The coronal views of femurs injected with both hydrogel groups, P (DWIVA peptide-functionalized hydrogel) and C (control, i.e., non-functionalized hydrogel), at two- and four-weeks post-injection are reported and analyzed (FIG. 32A). By week four, the peptide-functionalized P group had a significant growth advantage over the non-functionalized hydrogel C group. Trabeculae bone volume (FIG. 32B) was also measured, and while no significant difference in volume was found between the groups at week two, a substantial increase in trabeculae volume was observed by the fourth week. However, the number of trabeculae (FIG. 32C) was higher in the P group at both time intervals, with a more significant difference observed between the two groups at week two compared to week four. The thickness of trabeculae (FIG. 32D) was also greater in the peptide-functionalized P group at weeks two and four than in the non-functionalized hydrogel C group. Additionally, the peptide-functionalized P group exhibited further support for enhanced bone growth over time, as evidenced by a significantly lower trabecular spacing, particularly at week four (FIG. 32E).

Cortical bone morphology and morphometry were also evaluated for both groups, quantifying the differences in average cortical bone thickness, surface area, area of the periosteal envelope, and cortical area fraction. It should be noted that a substantial increase in the denser cortical bone is undesirable since it could impede the flexibility of newly formed trabecular tissue. Within this study, no significant differences were found in cortical bone formations between groups C and P at either time interval. Therefore, improper cortical bone was not observed to interfere with trabecular bone growth. Instead, the present study found that only trabeculae bone formation was greater in both hydrogel groups at four weeks post-injection, with significantly more growth observed in injured femurs treated with hydrogels coupled with the DWIVA peptide than those treated with hydrogel alone.

### Example 3-6: Inclusion of MSCs within Injectable DWIVA Hydrogels Enhances Mineralized Bone Formation

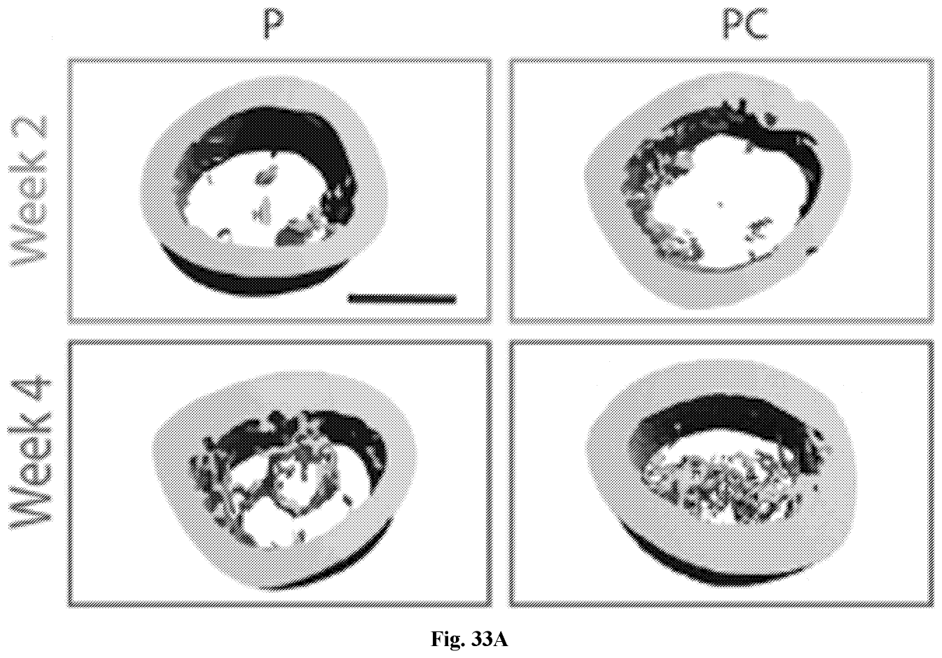

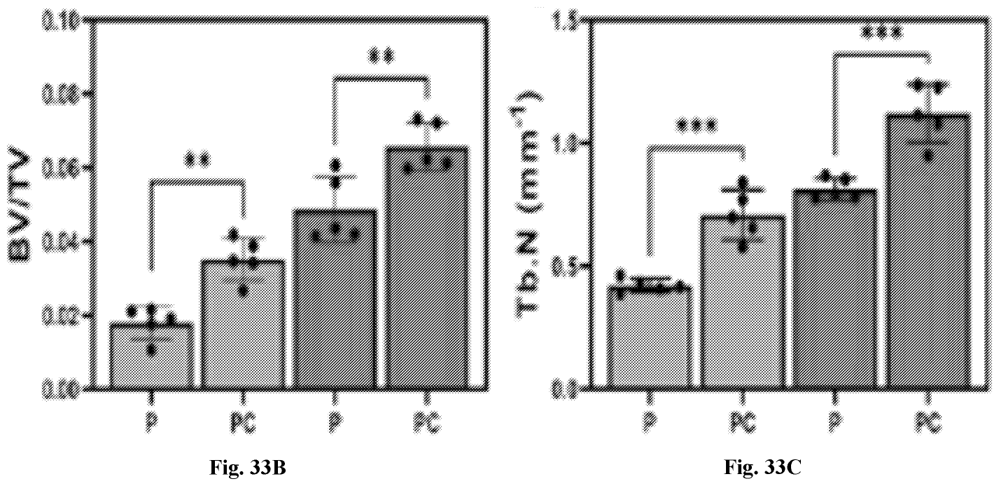

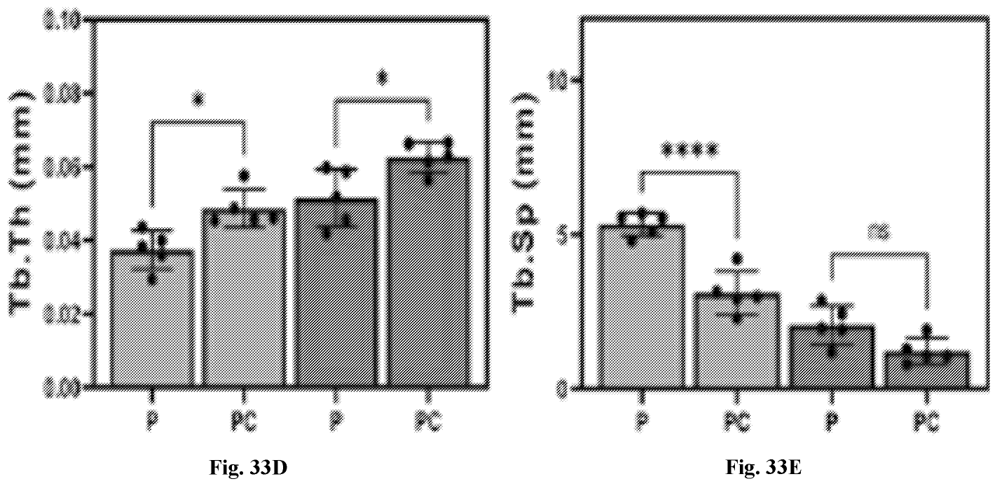

Treatment of MSC-laden DWIVA peptide hydrogels led to greater overall trabecular bone growth, as demonstrated by micro-CT imaging and quantitative data. For example, coronal views of treated femurs showed more trabecular growth in the PC group (MSC-laden peptide functionalized hydrogel group), with a significant increase in bone growth observed at four weeks post-injection (FIG. 33A). Additionally, there were significant increases reported in trabeculae volume, number, and thickness, as well as decreased trabeculae spacing from weeks two to four. Though the trabeculae volume of the peptide functionalized hydrogel only P group wasn't as high as that of the PC group, it did still increase over time (FIG. 33B). The number (FIG. 33C) and thickness (FIG. 33D) of trabeculae in the treated femur increased as a function of encapsulated MSCs, especially after four weeks. Interestingly, more significance is calculated for trabeculae number when compared to trabeculae thickness between both groups at both time intervals. Finally, the trabeculae spacing (FIG. 33E) of the P and PC groups was significantly different at two weeks, however, there was not a significant difference at four weeks. The average cortical bone thickness, surface area, periosteal envelope area, or cortical area fraction of groups P and PC were also not significantly different, similar to the cortical bone morphology and morphometry data observed between groups C and P.

### Example 3-7: DWIVA and MSCs Induce Increased Unmineralized Bone ECM Formation

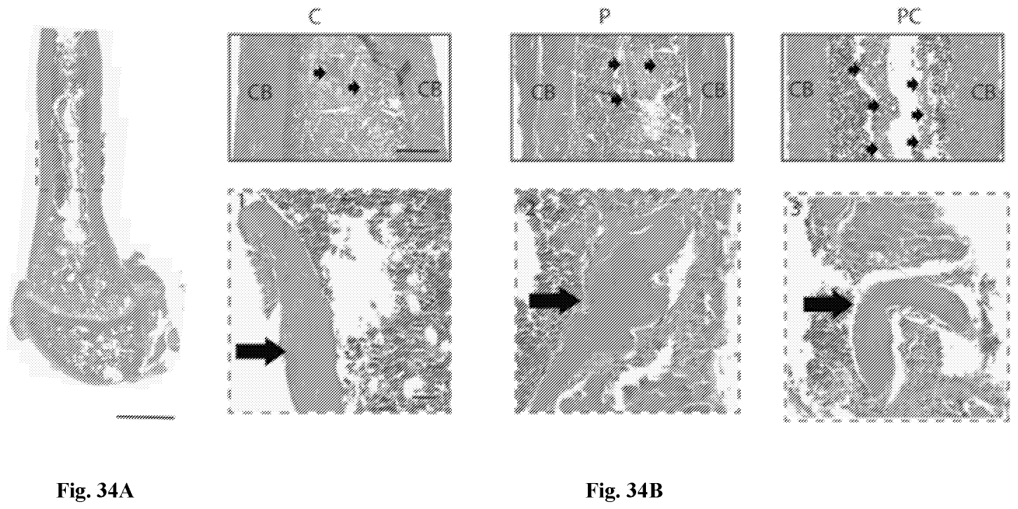

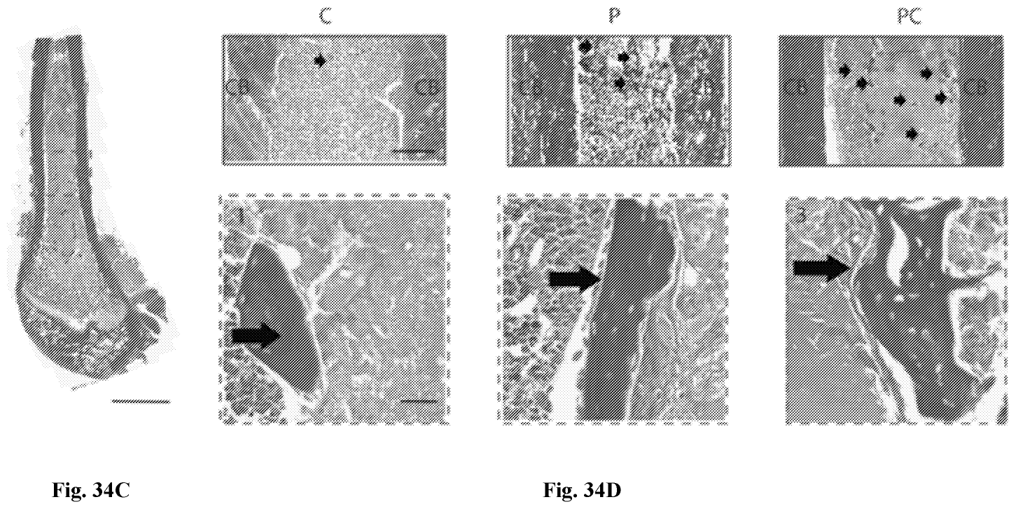

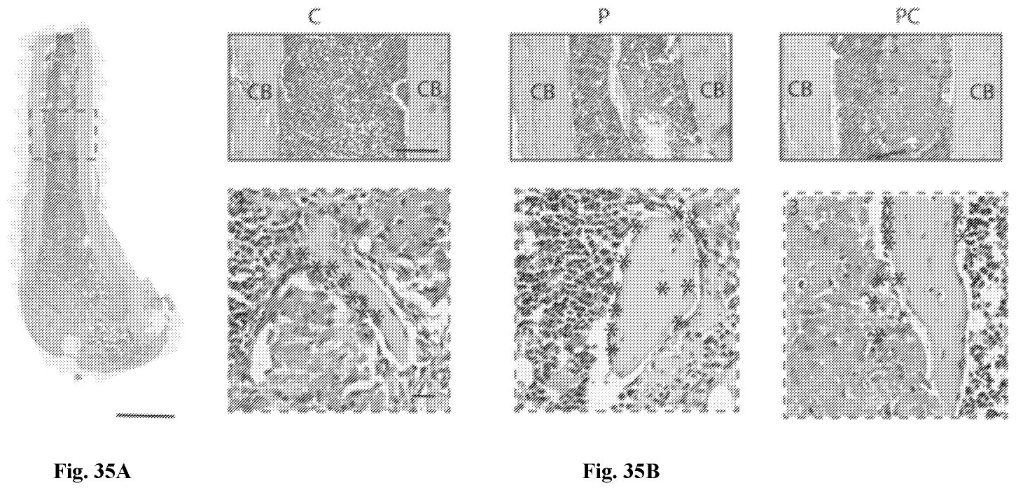

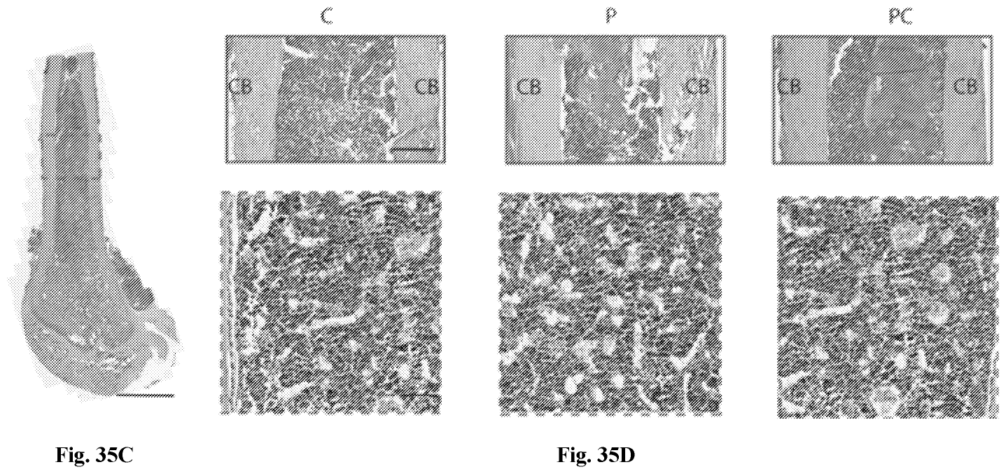

Histomorphometric analysis confirms that femurs treated with peptide-coupled hydrogels with encapsulated MSCs in the PC group exhibit greater trabecular bone growth, with significantly more growth than both the P and C groups. To histologically evaluate bone growth in groups C, P, and PC, H&E and Masson's Trichrome staining techniques were used on tissue sections of the extracted rat femurs. ALP and TRAP stains were then performed for the determination of osteoblast and osteoclast formations. Cortical bone (labelled as CB) exists as the outer layer that surrounds and supports the trabecular bone. Enlarged images of the PC group include arrows which indicate regions of trabecular bone growth (FIGS. 34A-34D) or asterisks which represent osteoblasts (brown) and osteoclast (orange) formations (FIGS. 35A-35D).

Within this study, an increased amount of osteoblast and osteoclast formations is seen in the PC group compared to other hydrogel groups. The balance between bone formation and bone resorption is critical in maintaining bone homeostasis, therefore an increase in both cell types could indicate that the bone is undergoing more active remodeling. The findings relative to this study suggest that encapsulating MSCs in hydrogels coupled with the peptide, DWIVA, has the potential to improve bone regeneration outcomes.

### Example 3-8: Quantitative Analysis of Osteoblast and Osteoclast Formation

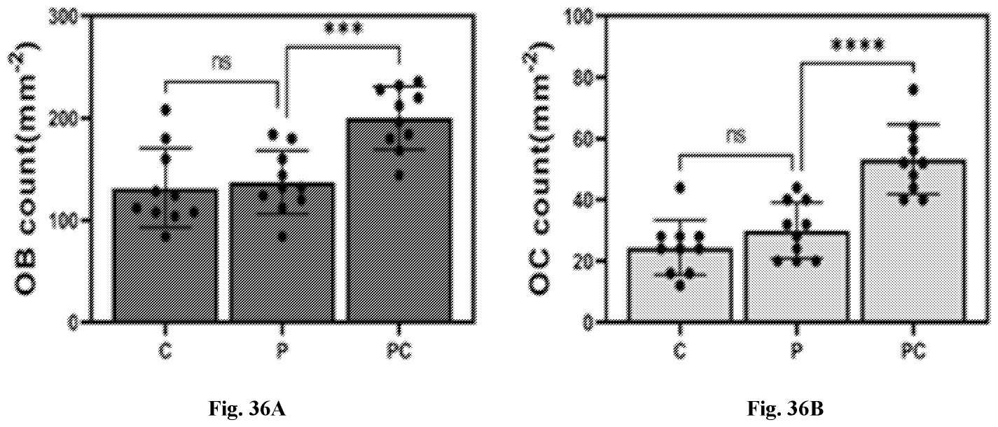

Quantitative analysis was performed to determine the number of osteoblast and osteoclast formation per unit area at four weeks post-injection (FIGS. 36A-36B). While no significant difference was observed between groups C and P, there was a significant increase in the number of both osteoblast and osteoclast formations in the PC group relative to the other groups. However, a significantly greater difference is observed between the number of osteoclasts in groups P and PC compared to the difference in the number of osteoblasts between the same groups. Conversely, there is no significant difference between all groups in both categories when assessed at two weeks post-injection. These findings suggest that time is an important factor in addition to the influence of MSCs on bone homeostasis in hydrogel-treated femurs.

### Example 3-9

The present study investigated the effects of a coupled peptide, DWIVA, and the regenerative potential of MSCs in a hydrogel scaffold implanted in injured rat femurs. By performing histomorphometric and morphometric analyses at weeks two and four post-treatment, it was found that co-delivery of MSCs within injectable DWIVA hydrogels resulted in a significant increase in mineralized bone, osteoid tissue, and bone remodeling. These findings suggest that the inclusion of MSCs in a hydrogel scaffold, functionalized with the DWIVA peptide, may enhance the regenerative potential of the hydrogel by improving bone repair outcomes. The quantitative data in this study provided further support for such efficacious therapies. For example, trabeculae volume, number, and thickness showed a significant increase in the group treated with hydrogels infused with MSCs, especially at week four post-injection. Importantly, unwanted formation of cortical bone, which is denser and more compact than trabecular bone, was not significant in any of the groups. An increase in cortical bone would not be desirable because it could lead to a loss of flexibility and natural movement, potentially leading to complications such as fractures or a reduced range of motion.

ALP and TRAP analyses were reported to have significantly greater osteoblast and osteoclast counts within trabecular bone when MSCs were co-delivered with the hydrogel to the injured femurs. As osteoblasts are cells responsible for bone formation and osteoclasts are cells responsible for bone resorption, a proper balance between them is crucial for healthy bone homeostasis. The higher prevalence of osteoblasts and osteoclasts seen in the femurs extracted from this group suggests that MSCs were playing a key role in promoting bone regeneration. This supports the hypothesis that MSCs enhance the therapeutic efficacy of the peptide-functionalized hydrogel.

Time was also a factor, where quantitative data shows only statistical evidence at week four for increased osteoid count in treated femurs. It is worth noting that exact timing and extent of bone formation can be influenced by several factors, including the severity of the injury, age, and overall health of the subject. Therefore, it is important to design effective interventions where both qualitative and quantitative data can be co-analyzed for a more comprehensive understanding of the mechanisms underlying the observed effects. Overall, the histomorphometric and morphometric analyses presented in this study provide compelling evidence that peptide-functionalized hydrogels promote greater bone repair when co-delivered with MSCs. These findings have important implications for the development of targeted and efficacious therapies for bone regeneration, as they highlight the potential of MSCs to enhance the therapeutic efficacy of biomaterial-based approaches.

### ENUMERATED EMBODIMENTS

In some aspects, the present invention is directed to the following non-limiting embodiments:

### Embodiment 1

A hydrogel comprising: a hydrogel polymer; a crosslinker crosslinking the hydrogel polymer; a biomolecule attached to the hydrogel polymer; and water, wherein the biomolecule is attached to the hydrogel polymer through an acrylic linker attached to a hydroxyl group in the hydrogel polymer.

### Embodiment 2

The hydrogel of Embodiment 1, wherein the hydrogel polymer comprises at least one selected from the group consisting of an alginate polymer, an α,β-poly(N-hydroxyethyl)-DL-aspartamide polymer, a chitosan polymer, a chondroitin sulfate polymer, a collagen/gelatin polymer, an elastin polymer, a fibrin polymer, a heparin polymer, a hyaluronic acid polymer, and a poly(vinyl alcohol) polymer.

### Embodiment 3

The hydrogel of any one of Embodiments 1-2, wherein the amount of the hydrogel polymer in the hydrogel ranges from about 1% w/v to about 10% w/v based on a total volume of the hydrogel.

### Embodiment 4

The hydrogel of any one of Embodiments 1-3, wherein the crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

### Embodiment 5

The hydrogel of any one of Embodiments 1-4, wherein the molar % of repeating units in the hydrogel polymer that are directly attached to the crosslinker ranges from about 10% to about 60%.

### Embodiment 6

The hydrogel of any one of Embodiments 1-5, wherein the acrylic linker is attached to the hydroxyl group in the hydrogel polymer via an esterification reaction between an acrylic anhydride and the hydroxyl group.

### Embodiment 7

The hydrogel of any one of Embodiments 1-6, wherein the biomolecule is attached to the acrylic linker via a Michael addition reaction between a nucleophilic group in the biomolecule and an alkenyl group in the acrylic linker.

### Embodiment 8

The hydrogel of Embodiment 7, wherein the nucleophilic group comprises a thiol group.

### Embodiment 9

The hydrogel of any one of Embodiments 1-8, wherein the biomolecule comprises a protein or a peptide.

### Embodiment 10

The hydrogel of any one of Embodiments 1-9, wherein the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

### Embodiment 11

The hydrogel of any one of Embodiments 9-10, wherein the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

### Embodiment 12

The hydrogel of any one of Embodiments 1-11, wherein the concentration of the biomolecule in the hydrogel ranges from about 0.05 mM to about 10 mM.

### Embodiment 13

The hydrogel of any one of Embodiments 1-12, wherein the storage modulus of the hydrogel at 37° C. or 25° C. ranges from about 500 Pa to about 25,000 Pa.

### Embodiment 14

The hydrogel of any one of Embodiments 1-13, wherein the elastic modulus of the hydrogel at 37° C. or 25° C. ranges from about 1 kPa to about 75 kPa.

### Embodiment 15

The hydrogel of any one of Embodiments 1-14, wherein the storage modulus of the hydrogel is higher than the loss modulus of the hydrogel at 37° C. or 25° C.

### Embodiment 16

The hydrogel of any one of Embodiments 1-15, wherein one of the following applies: (a) the hydrogel does not comprise a cell; (b) the hydrogel comprises a cell, optionally a stem cell, optionally a mesenchymal stem cell (MSC).

### Embodiment 17

A hyaluronic acid (HA)-based hydrogel, comprising: a first HA polymer comprising a first crosslinker; a second HA polymer comprising a second crosslinker; a biomolecule attached to the first HA polymer or the second HA polymer through an acrylic linker attached to a hydroxyl group in the first HA polymer or the second HA polymer; and water.

### Embodiment 18

The HA-based hydrogel of Embodiment 17, wherein the sum of the amount of the first polymer and the amount of the second polymer in the HA-based hydrogel ranges from about 1% w/v to about 10% w/v based on the total volume of the HA-based hydrogel.

### Embodiment 19

The HA-based hydrogel of any one of Embodiments 17-18, wherein the first crosslinker and the second crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

### Embodiment 20

The HA-based hydrogel of any one of Embodiments 17-19, wherein the molar % of repeating units in the first HA polymer and the second HA polymer that are directly attached to the crosslinker ranges from about 10% to about 60%.

### Embodiment 21

The HA-based hydrogel of any one of Embodiments 17-20, wherein the acrylic linker is attached to the hydroxyl group in the first HA polymer or the second HA polymer via an esterification reaction between an acrylic anhydride and the hydroxyl group.

### Embodiment 22

The HA-based hydrogel of any one of Embodiments 17-21, wherein the biomolecule is attached to the acrylic linker via a Michael addition reaction between a nucleophilic group in the biomolecule and an alkenyl group in the acrylic linker.

### Embodiment 23

The HA-based hydrogel of Embodiment 22, wherein the nucleophilic group comprises a thiol group.

### Embodiment 24

The HA-based hydrogel of any one of Embodiments 16-23, wherein the biomolecule comprises a protein or a peptide.

### Embodiment 25

The HA-based hydrogel of any one of Embodiments 16-24, wherein the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

### Embodiment 26

The HA-based hydrogel of any one of Embodiments 24-25, wherein the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

### Embodiment 27

The HA-based hydrogel of any one of Embodiments 17-26, wherein the concentration of the biomolecule in the HA-based hydrogel ranges from about 0.05 mM to about 10 mM.

### Embodiment 28

The HA-based hydrogel of any one of Embodiments 17-27, wherein the storage modulus of the HA-based hydrogel at 37° C. or 25° C. ranges from about 500 Pa to about 25,000 Pa.

### Embodiment 29

The HA-based hydrogel of any one of Embodiments 17-28, wherein the elastic modulus of the HA-based hydrogel at 37° C. or 25° C. ranges from about 1 kPa to about 75 kPa.

### Embodiment 30

The HA-based hydrogel of any one of Embodiments 17-29, wherein the storage modulus of the HA-based hydrogel is higher than the loss modulus of the HA-based hydrogel at 37° C. or 25° C.

### Embodiment 31

The HA-based hydrogel of any one of Embodiments 17-30, wherein one of the following applies: (a) the HA-based hydrogel does not comprise a cell; (b) the HA-based hydrogel comprises a cell, optionally a stem cell, optionally a mesenchymal stem cell (MSC).

### Embodiment 32

A method of preparing a HA-based hydrogel, the method comprising: attaching a first crosslinker to a first HA polymer; attaching a second crosslinker to a second HA polymer; attaching an acrylic linker to a hydroxyl group of the first HA polymer or the second HA polymer; attaching a biomolecule to the acrylic link; mixing the first HA polymer attached with the first crosslinker, the second HA polymer attached with the second crosslinker and water; crosslinking the first crosslinker and the second crosslinker.

### Embodiment 33

The method of Embodiment 32, wherein the sum of the amount of the first polymer and the amount of the second polymer in the HA-based hydrogel ranges from about 1% w/v to about 10% w/v based on the total volume of the mixture of the first HA polymer, the second HA polymer, and the water.

### Embodiment 34

The method of any one of Embodiments 32-33, wherein the first crosslinker and the second crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

### Embodiment 35

The method of any one of Embodiments 32-34, wherein the molar percentage of repeating units in the first HA polymer and the second HA polymer that are directly attached to the crosslinker ranges from about 10% to about 60%.

### Embodiment 36

The method of any one of Embodiments 32-35, wherein attaching the acrylic linker to the hydroxyl group of the first HA polymer or the second HA polymer comprises attaching the acrylic linker to the hydroxyl group via an esterification reaction between an acrylic anhydride and the hydroxyl group.

### Embodiment 37

The method of any one of Embodiments 32-36, wherein attaching the biomolecule to the acrylic linker comprises attaching a nucleophilic group the biomolecule to an alkenyl group of the acrylic linker via a Michael addition reaction.

### Embodiment 38

The method of Embodiment 37, wherein the nucleophilic group comprises a thiol group.

### Embodiment 39

The method of any one of Embodiments 32-38, wherein the biomolecule comprises a protein or a peptide.

### Embodiment 40

The method of any one of Embodiments 32-39, wherein the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

### Embodiment 41

The method of any one of Embodiments 39-40, wherein the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

### Embodiment 42

The method of any one of Embodiments 32-41, wherein the concentration of the biomolecule in the mixture of the first HA polymer, the second HA polymer and the water ranges from about 0.05 mM to about 10 mM.

### Embodiment 43

The method of any one of Embodiments 32-42, wherein the storage modulus of the HA-based hydrogel at 37° C. or 25° C. ranges from about 500 Pa to about 25,000 Pa.

### Embodiment 44

The method of any one of Embodiments 32-43, wherein the elastic modulus of the HA-based hydrogel at 37° C. or 25° C. ranges from about 1 kPa to about 75 kPa.

### Embodiment 45

The method of any one of Embodiment 32-44, wherein the storage modulus of the HA-based hydrogel is higher than the loss modulus of the HA-based hydrogel at 37° C. or 25° C.

### Embodiment 46

The method of any one of Embodiments 32-45, wherein one of the following applies: (a) the mixture of the first HA polymer, the second HA polymer and the water does not comprise a cell, (b) the mixture of the first HA polymer, the second HA polymer and the water comprises a cell, such as a stem cell.

### Embodiment 47

A method of promoting regeneration of a tissue in a subject in need thereof, the method comprising: placing the hydrogel of any one of Embodiments 1-16 or the HA-based hydrogel of any one of Embodiments 17-31 at a site in need of regeneration in the subject.

### Embodiment 48

The method of Embodiment 47, wherein placing the hydrogel or the HA-based hydrogel at the site comprises forming the hydrogel or the HA-based hydrogel at the site.

### Embodiment 49

The method of Embodiment 48, wherein forming the hydrogel or the HA-based hydrogel at the site comprises injecting a liquid mixture for forming the hydrogel or the HA-based hydrogel into the site.

### Embodiment 50

The method of Embodiment 49, wherein the mixture undergoes crosslinking and gelates (gels) to form the hydrogel or the HA-based hydrogel spontaneously at the site.

### Embodiment 51

The method of any one of Embodiments 47-50, wherein the tissue is a bone tissue, a cartilage tissue, or combinations thereof.

### Embodiment 52

The method of any one of Embodiments 47-51, wherein the biomolecule attached to the hydrogel polymer, the first HA polymer or the second HA polymer is bone morphogenetic protein 2 (BMP-2), or a functional mimicking peptide of BMP-2.

### Embodiment 53

The method of Embodiment 52, wherein the biomolecule is the functional mimicking peptide of BMP-2, and wherein the functional mimicking peptide of BMP-2 comprises at least one of the following amino acid sequences: GCGGGDWIVAG (SEQ ID NO: 8), NSVNSKIPKACCVPTELSAI (SEQ ID NO:9), or KIPKASSVPTELSAISTLYL (SEQ ID NO: 10).

### Embodiment 54

The method of any one of Embodiment 47-53, wherein the hydrogel or the HA-based hydrogel is placed in a femur of the subject.

### Embodiment 55

The method of any one of Embodiments 47-54, wherein the subject is suffering from osteoporosis.

### Embodiment 56

The method of any one of Embodiments 47-55, wherein the subject is a mammal, optionally a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A hydrogel comprising:
a hydrogel polymer;
a crosslinker crosslinking the hydrogel polymer;
a biomolecule attached to the hydrogel polymer; and
water,
wherein the biomolecule is attached to the hydrogel polymer through an acrylic linker attached to a hydroxyl group in the hydrogel polymer.

2. The hydrogel of claim 1, wherein the hydrogel polymer comprises at least one selected from the group consisting of an alginate polymer, an α,β-poly(N-hydroxyethyl)-DL-aspartamide polymer, a chitosan polymer, a chondroitin sulfate polymer, a collagen/gelatin polymer, an elastin polymer, a fibrin polymer, a heparin polymer, a hyaluronic acid polymer, and a poly(vinyl alcohol) polymer.

3. The hydrogel of claim 1, wherein the amount of the hydrogel polymer in the hydrogel ranges from about 1% w/v to about 10% w/v based on a total volume of the hydrogel.

4. The hydrogel of claim 1, wherein the crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

5. The hydrogel of claim 1, wherein the molar % of repeating units in the hydrogel polymer that are directly attached to the crosslinker ranges from about 10% to about 60%.

6. The hydrogel of claim 1, wherein the acrylic linker is attached to the hydroxyl group in the hydrogel polymer via an esterification reaction between an acrylic anhydride and the hydroxyl group.

7. The hydrogel of claim 1, wherein the biomolecule is attached to the acrylic linker via a Michael addition reaction between a nucleophilic group in the biomolecule and an alkenyl group in the acrylic linker,
optionally wherein the nucleophilic group comprises a thiol group.

8. (canceled)

9. The hydrogel of claim 1, wherein the biomolecule comprises a protein or a peptide,
optionally wherein the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

10. (canceled)

11. The hydrogel of claim 9, wherein the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

12. The hydrogel of claim 1, wherein at least one of the following applies:
(a) the concentration of the biomolecule in the hydrogel ranges from about 0.05 mM to about 10 mM;
(b) the storage modulus of the hydrogel at 37° C. or 25° C. ranges from about 500 Pa to about 25,000 Pa;
(c) the elastic modulus of the hydrogel at 37° C. or 25° C. ranges from about 1 kPa to about 75 kPa;
(d) the storage modulus of the hydrogel is higher than the loss modulus of the hydrogel at 37° C. or 25° C.;
(e) the hydrogel does not comprise a cell,
(f) the hydrogel comprises a cell, optionally a stem cell, optionally a mesenchymal stem cell (MSC).

13-16. (canceled)

17. A hyaluronic acid (HA)-based hydrogel, comprising:
a first HA polymer comprising a first crosslinker;
a second HA polymer comprising a second crosslinker;
a biomolecule attached to the first HA polymer or the second HA polymer through an acrylic linker attached to a hydroxyl group in the first HA polymer or the second HA polymer; and
water.

18. The HA-based hydrogel of claim 17, wherein the sum of the amount of the first polymer and the amount of the second polymer in the HA-based hydrogel ranges from about 1% w/v to about 10% w/v based on the total volume of the HA-based hydrogel.

19. The HA-based hydrogel of claim 17, wherein the first crosslinker and the second crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair.

20. The HA-based hydrogel of claim 17, wherein the molar % of repeating units in the first HA polymer and the second HA polymer that are directly attached to the crosslinker ranges from about 10% to about 60%.

21. The HA-based hydrogel of claim 17, wherein the acrylic linker is attached to the hydroxyl group in the first HA polymer or the second HA polymer via an esterification reaction between an acrylic anhydride and the hydroxyl group.

22. The HA-based hydrogel of claim 17, wherein the biomolecule is attached to the acrylic linker via a Michael addition reaction between a nucleophilic group in the biomolecule and an alkenyl group in the acrylic linker,
optionally wherein the nucleophilic group comprises a thiol group.

23. (canceled)

24. The HA-based hydrogel of claim 17, wherein the biomolecule comprises a protein or a peptide,
optionally wherein the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

25. (canceled)

26. The HA-based hydrogel of claim 24, wherein the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

27. The HA-based hydrogel of claim 17, wherein at least one of the following applies:
(a) the concentration of the biomolecule in the HA-based hydrogel ranges from about 0.05 mM to about 10 mM;
(b) the storage modulus of the HA-based hydrogel at 37° C. or 25° C. ranges from about 500 Pa to about 25,000 Pa;
(c) the elastic modulus of the HA-based hydrogel at 37° C. or 25° C. ranges from about 1 kPa to about 75 kPa;
(d) the storage modulus of the HA-based hydrogel is higher than the loss modulus of the HA-based hydrogel at 37° C. or 25° C.;
(e) the HA-based hydrogel does not comprise a cell;
(f) the HA-based hydrogel comprises a cell, optionally a stem cell, optionally a mesenchymal stem cell (MSC).

28-31. (canceled)

32. A method of preparing a HA-based hydrogel, the method comprising:
attaching a first crosslinker to a first HA polymer;
attaching a second crosslinker to a second HA polymer;
attaching an acrylic linker to a hydroxyl group of the first HA polymer or the second HA polymer;
attaching a biomolecule to the acrylic link;
mixing the first HA polymer attached with the first crosslinker, the second HA polymer attached with the second crosslinker and water;
crosslinking the first crosslinker and the second crosslinker.

33. The method of claim 32, wherein at least one of the following applies:
(a) the sum of the amount of the first polymer and the amount of the second polymer in the HA-based hydrogel ranges from about 1% w/v to about 10% w/v based on the total volume of the mixture of the first HA polymer, the second HA polymer, and the water;
(b) the first crosslinker and the second crosslinker comprises a pair of a diene and a dienophile that undergoes a catalyst-free Diels-Alder reaction, optionally a furan-dimaleimide pair, a tetrazine-di-norbornene pair, or a tetrazine-norbornene pair;
(c) the molar percentage of repeating units in the first HA polymer and the second HA polymer that are directly attached to the crosslinker ranges from about 10% to about 60%.

34-35. (canceled)

36. The method of claim 32, wherein attaching the acrylic linker to the hydroxyl group of the first HA polymer or the second HA polymer comprises attaching the acrylic linker to the hydroxyl group via an esterification reaction between an acrylic anhydride and the hydroxyl group.

37. The method of claim 32, wherein attaching the biomolecule to the acrylic linker comprises attaching a nucleophilic group the biomolecule to an alkenyl group of the acrylic linker via a Michael addition reaction, optionally wherein the nucleophilic group comprises a thiol group.

38. (canceled)

39. The method of claim 32, wherein the biomolecule comprises a protein or a peptide,
optionally wherein the biomolecule comprises an extracellular protein or a functional mimicking peptide thereof, a growth factor or a functional mimicking peptide thereof, an angiogenic protein or a functional mimicking peptide thereof, or a glycosaminoglycan (GAG)-binding peptide.

40. (canceled)

41. The method of claim 39, wherein the protein or peptide comprises at least one cysteine residue, and wherein the protein or peptide is attached to the acrylic linker via a thiol Michael addition reaction between a thiol group of the at least one cysteine residue and an alkenyl group in the acrylic linker.

42. The method of claim 32, wherein at least one the following applies:
(a) the concentration of the biomolecule in the mixture of the first HA polymer, the second HA polymer and the water ranges from about 0.05 mM to about 10 mM;
(b) the storage modulus of the HA-based hydrogel at 37° C. or 25° C. ranges from about 500 Pa to about 25,000 Pa;
(c) the elastic modulus of the HA-based hydrogel at 37° C. or 25° C. ranges from about 1 kPa to about 75 kPa;
(d) the storage modulus of the HA-based hydrogel is higher than the loss modulus of the HA-based hydrogel at 37° C. or 25° C.;
(e) the mixture of the first HA polymer, the second HA polymer and the water does not comprise a cell;
(f) the mixture of the first HA polymer, the second HA polymer and the water comprises a cell, optionally as a stem cell.

43-46. (canceled)

47. A method of promoting regeneration of a tissue in a subject in need thereof, the method comprising:
placing a hydrogel or a hyaluronic acid (HA)-based hydrogel at a site in need of regeneration in the subject,
wherein the hydrogel comprises:
a hydrogel polymer;
a crosslinker crosslinking the hydrogel polymer;
a biomolecule attached to the hydrogel polymer; and
water,
wherein the biomolecule is attached to the hydrogel polymer through an acrylic linker attached to a hydroxyl group in the hydrogel polymer; and
wherein the HA-based hydrogel comprises:
a first HA polymer comprising a first crosslinker;
a second HA polymer comprising a second crosslinker;
a biomolecule attached to the first HA polymer or the second HA polymer through an acrylic linker attached to a hydroxyl group in the first HA polymer or the second HA polymer; and
water.

48. The method of claim 47, wherein at least one of the following applies:
(a) placing the hydrogel or the HA-based hydrogel at the site comprises forming the hydrogel or the HA-based hydrogel at the site;
(b) forming the hydrogel or the HA-based hydrogel at the site comprises injecting a liquid mixture for forming the hydrogel or the HA-based hydrogel into the site;
(c) the tissue is a bone tissue, a cartilage tissue, or combinations thereof,
(d) the biomolecule attached to the hydrogel polymer, the first HA polymer or the second HA polymer is bone morphogenetic protein 2 (BMP-2), or a functional mimicking peptide of BMP-2;
(e) the hydrogel or the HA-based hydrogel is placed in a femur of the subject;
(f) the subject is suffering from osteoporosis;
(g) the subject is a mammal, optionally a human.

49. (canceled)

50. The method of claim 48, wherein at least one of the following applies:
in (b) the mixture undergoes crosslinking and gelates (gels) to form the hydrogel or the HA-based hydrogel spontaneously at the site;
in (d) the biomolecule is the functional mimicking peptide of BMP-2, and wherein the functional mimicking peptide of BMP-2 comprises at least one of the following amino acid sequences:
(SEQ ID NO: 8)
GCGGGDWIVAG,
(SEQ ID NO: 9)
NSVNSKIPKACCVPTELSAI,
or
(SEQ ID NO: 10)
KIPKASSVPTELSAISTLYL

51-56. (canceled)
